(19) United States
(12) Patent Application Publication
(10) Pub. No.: US 2026/0265356 A1
(43) Pub. Date: Sep. 10, 2026

(54) COMPLEMENT PATHWAY INHIBITOR POLYPEPTIDES AND METHODS OF USE THEREOF

(71) Applicant: Otsuka Pharmaceutical Co., Ltd., Osaka (JP)

(72) Inventors: Boopathy Ramakrishnan, Waltham, MA (US); Bharat Chaganty, Waltham, MA (US); Hedy Adari, Waltham, MA (US); Karthik Viswanathan, Waltham, MA (US)

(21) Appl. No.: 19/558,993

(22) Filed: Mar. 6, 2026

Related U.S. Application Data
(60) Provisional application No. 63/768,033, filed on Mar. 6, 2025.

(51) Int. Cl. C07K 16/18; A61K 38/00; A61P 37/06; C07K 14/705; C12N 15/86

(52) U.S. Cl. CPC C07K 16/18; A61P 37/06; C07K 14/70596; C12N 15/86; A61K 38/00; C07K 2317/52; C07K 2317/565; C07K 2317/569; C07K 2319/00; C12N 2750/14143

(57) ABSTRACT

The present invention provides, among other things, a polypeptide that is capable of binding to complement C3b (C3b), wherein the polypeptide comprises at least (A) a first moiety and (B) a second moiety, wherein the first moiety comprises a variable domain capable of binding to C3b, and the second moiety comprises an amino acid sequence of membrane cofactor protein (MCP) or a variant thereof.

### CROSS REFERENCE TO SEQUENCE LISTING

The present application is filed along with a Sequence Listing submitted electronically in XML format. The Sequence Listing file named “SVI-019WO1.xml” created on Mar. 5, 2026, is 62,123 bytes in size; the content of which is incorporated herein by reference in its entirety.

### CROSS REFERENCE TO RELATED APPLICATIONS

The present application claims priority to, and the benefit of U.S. Provisional Patent Application No. 63/768,033, filed on Mar. 6, 2025; the entirety of which is hereby incorporated by reference for all purpose.

### BACKGROUND

The complement system is a part of the innate immune system, playing a pivotal role in the clearing of the invading microbes, as well as in removal of immune complexes and apoptotic bodies. The complement is also an important bridge between innate and adaptive immune response. It is not surprising that its pivotal role in tuning both innate and adaptive immune response evolved together with its fine regulatory role, and that impairment of such regulation impacts all major organs, causing many diseases.

The complement system has three pathways of activation: the classical, the alternative and the lectin pathway. These three pathways follow a common cascade mechanism; activated proteins cleave the zymogens that then get activated to cleave and activate the next target in the cascade. All three pathways converge at the activation of complement component C3, which includes generation of C3 convertase. In addition to the central complement component 3 (C3), the alternative pathway also includes other key components including complement factor D (FD), complement factor B (FB), and properdin (P).

Several endogenous complement regulators, such as surface proteins complement receptor 1 (CR1 or CD35), and membrane cofactor protein (MCP or CD46), and fluid-phase proteins complement factor I (FI), and complement factor H (FH), can modulate the activity of the complement alternative pathway (AP). Alternative pathway is often the primary driver of overactivation of complement cascade. Alternative pathway dysregulation is implicated in a number of diseases such as polycystic kidney disease, PNH, aHUS, macular degeneration and C3 glomerulopathy.

### SUMMARY OF THE INVENTION

The present invention provides, among other things, C3b inhibitor polypeptides comprising a variable domain capable of binding to C3b and an amino acid sequence of membrane cofactor protein (MCP). In particular, the polypeptides of the present invention selectively binds to C3b with high affinity and inhibit alternative pathway of complement system without affecting the initiation of classical and lectin pathways. Without wishing to be bound by any particular theory, it is contemplated that the present invention encompasses C3b inhibitor polypeptides and therapeutic proteins thereof that inhibit alternative pathway specifically and those that inhibit the downstream components and steps shared by both alternative pathway and classic pathway through, for example, inhibition of C3b, the central and activated component of alternative, classic and lectin pathways. As described herein, the present invention is, in part, based on the identification of a new class of C3b inhibitor polypeptides that (i) irreversibly inhibit alternative pathway; (ii) inhibit the C3 convertase formation; (iii) target both solid phase and fluid phase alternative pathway C3 convertase activity; (iv) block C3b interaction with factor B (fB); (v) inhibit activity specific to the alternative pathway (AP); (vi) have favorable biophysical and pharmacokinetic properties to allow for prolonged therapeutic effect; and/or (vii) inhibit stabilization of C3 convertase by C3bNef autoantibodies. Inventive polypeptides capable of binding to C3b of the present invention promise a more potent treatment of C3b mediated diseases and disorders.

In one aspect, the present invention provides, among other things, a polypeptide that is capable of binding to complement C3b (C3b), wherein the polypeptide comprises at least (A) a first moiety and (B) a second moiety, wherein the first moiety comprises a variable domain capable of binding to C3b, and the second moiety comprises an amino acid sequence of membrane cofactor protein (MCP) or a variant thereof.

In some embodiments, the variable domain of the first moiety comprises a heavy complementary-determining region 1 (HCDR1), a heavy complementary-determining region 2 (HCDR2) and a heavy complementary-determining region 3 (HCDR3), wherein the HCDR1, HCDR2 and HCDR3 comprises the sequence of SEQ ID NO: 16, 17, and 18 respectively. In some embodiments, the variable domain of the first moiety comprises HCDR1, HCDR2 and HCDR3, wherein the HCDR1, HCDR2 and HCDR3 comprises the sequence of SEQ ID NO: 19, 20, and 18 respectively.

In some embodiments, the variable domain of the first moiety comprises HCDR1, HCDR2 and HCDR3, wherein the HCDR1, HCDR2 and HCDR3 comprises the sequence of SEQ ID NO: 16, 26, and 22, respectively. In some embodiments, the variable domain of the first moiety comprises HCDR1, HCDR2 and HCDR3, wherein the HCDR1, HCDR2 and HCDR3 comprises the sequence of SEQ ID NO: 19, 27, and 22, respectively.

In some embodiments, the first moiety comprises the sequence of SEQ ID NO: 13. In some embodiments, the first moiety comprises the sequence of SEQ ID NO: 10. In some embodiments, the first moiety comprises the sequence of SEQ ID NO: 11. In some embodiments, the first moiety comprises the sequence of SEQ ID NO: 12.

In some embodiments, the second moiety has at least 90% sequence identity to the sequence of SEQ ID NO: 14. In some embodiments, the second moiety has at least 91% sequence identity to the sequence of SEQ ID NO:14. In some embodiments, the second moiety has at least 92% sequence identity to the sequence of SEQ ID NO: 14. In some embodiments, the second moiety has at least 93% sequence identity to the sequence of SEQ ID NO: 14. In some embodiments, the second moiety has at least 94% sequence identity to the sequence of SEQ ID NO: 14. In some embodiments, the second moiety has at least 95% sequence identity to the sequence of SEQ ID NO: 14. In some embodiments, the second moiety has at least 96% sequence identity to the sequence of SEQ ID NO: 14. In some embodiments, the second moiety has at least 97% sequence identity to the sequence of SEQ ID NO: 14. In some embodiments, the second moiety has at least 98% sequence identity to the sequence of SEQ ID NO: 14. In some embodiments, the second moiety has at least 99% sequence identity to the sequence of SEQ ID NO: 14.

In some embodiments, the second moiety has at least 90% sequence identity to the sequence of SEQ ID NO: 9. In some embodiments, the second moiety has at least 91% sequence identity to the sequence of SEQ ID NO: 9. In some embodiments, the second moiety has at least 92% sequence identity to the sequence of SEQ ID NO: 9. In some embodiments, the second moiety has at least 93% sequence identity to the sequence of SEQ ID NO: 9. In some embodiments, the second moiety has at least 94% sequence identity to the sequence of SEQ ID NO: 9. In some embodiments, the second moiety has at least 95% sequence identity to the sequence of SEQ ID NO: 9. In some embodiments, the second moiety has at least 96% sequence identity to the sequence of SEQ ID NO: 9. In some embodiments, the second moiety has at least 97% sequence identity to the sequence of SEQ ID NO: 9. In some embodiments, the second moiety has at least 98% sequence identity to the sequence of SEQ ID NO: 9. In some embodiments, the second moiety has at least 99% sequence identity to the sequence of SEQ ID NO: 9.

In some embodiments, the second moiety comprises a mutation of S177R to the sequence of SEQ ID NO: 9.

In some embodiments, the second moiety comprises the sequence of SEQ ID NO: 15.

In some embodiments, the first moiety is indirectly linked to the second moiety.

In some embodiments, the first moiety is directly linked to the second moiety.

In some embodiments, the first moiety is directly linked to the second moiety via a linker (Linker 1).

In some embodiments, the C-terminus of the first moiety is linked to the N-terminus of the second moiety.

In some embodiments, the N-terminus of the first moiety is linked to the C-terminus of the second moiety.

In some embodiments, from the N-terminus to the C-terminus, the first moiety comprising the sequence of SEQ ID NO: 10 linked to the second moiety comprising the sequence of SEQ ID NO: 9. In some embodiments, from the C-terminus to the N-terminus, the first moiety comprising the sequence of SEQ ID NO: 10 linked to the second moiety comprising the sequence of SEQ ID NO: 9.

In some embodiments, from the N-terminus to the C-terminus, the first moiety comprising the sequence of SEQ ID NO: 13 is linked to the second moiety comprising the sequence of SEQ ID NO: 9. In some embodiments, from the C-terminus to the N-terminus, the first moiety comprising the sequence of SEQ ID NO: 13 is linked to the second moiety comprising the sequence of SEQ ID NO: 9.

In some embodiments, from the N-terminus to the C-terminus, the first moiety comprising the sequence of SEQ ID NO: 10 is linked to the second moiety comprising the sequence of SEQ ID NO: 15. In some embodiments, from the C-terminus to the N-terminus, the first moiety comprising the sequence of SEQ ID NO: 10 is linked to the second moiety comprising the sequence of SEQ ID NO: 15.

In some embodiments, from the N-terminus to the C-terminus, the first moiety comprising the sequence of SEQ ID NO: 13 is linked to the second moiety comprising the sequence of SEQ ID NO: 15. In some embodiments, from the C-terminus to the N-terminus, the first moiety comprising the sequence of SEQ ID NO: 13 is linked to the second moiety comprising the sequence of SEQ ID NO: 15.

In some embodiments, Linker 1 is 20 amino acids or less. In some embodiments, Linker 1 is 15 amino acids or less.

In some embodiments, Linker 1 comprises GGGGS (SEQ ID NO: 37). In some embodiments, Linker 1 comprises GGGGSGGGGS (SEQ ID NO: 38). In some embodiments, Linker 1 comprises GGGGSGGGGSGGGGS (SEQ ID NO: 39). In some embodiments, Linker 1 comprises GGGGSGGGGSGGGGSGGGGS (SEQ ID NO: 40).

In some embodiments, the polypeptide comprises the sequence of SEQ ID NO: 5. In some embodiments, the polypeptide comprises the sequence of SEQ ID NO: 8.

In some embodiments, the polypeptide further comprises (C) a third moiety comprising a half-life extension domain.

In some embodiments, the third moiety comprises an Fc domain.

In some embodiments, the Fc domain is derived from IgG1, IgG2, or IgG4. In some embodiments, the Fc domain is derived from IgG1. In some embodiments, the Fc domain is derived from IgG2. In some embodiments, the Fc domain is derived from IgG4.

In some embodiments, the Fc domain comprises an amino acid modification of T307Q. In some embodiments, the Fc domain comprises an amino acid modification of Q311V. In some embodiments, the Fc domain comprises an amino acid modification of A378V. In some embodiments, the Fc domain comprises an amino acid modification of T307Q, Q311V and A378V.

In some embodiments, the Fc domain comprises an amino acid modification of F234V. In some embodiments, the Fc domain comprises an amino acid modification of L235E. In some embodiments, the Fc domain comprises an amino acid modification of D265G. In some embodiments, the Fc domain comprises an amino acid modification of F234V, L235E and D265G.

In some embodiments, the Fc domain comprises an amino acid modification of S228P.

In some embodiments, the N-terminus of the first moiety is linked to the C-terminus of the third moiety. In some embodiments, the C-terminus of the first moiety is linked to the N-terminus of the third moiety. As non-limiting examples, the polypeptide comprises from the N-terminus to the C-terminus, the Fc domain, the first moiety and the second moiety.

In some embodiments, the polypeptide comprises, from the N-terminus to the C-terminus, the third moiety comprising the Fc domain having the amino acid modifications of a) T307Q, Q311V and A378V; b) F234V, L235E and D265G; and c) S228P, the first moiety comprising the sequence of SEQ ID NO: 10, and the second moiety comprising the sequence of SEQ ID NO: 9.

In some embodiments, the polypeptide comprises, from the N-terminus to the C-terminus, the third moiety comprising the Fc domain having the amino acid modifications of a)T307Q, Q311V and A378V; and b) F234V, L235E and D265G, the first moiety comprising the sequence of SEQ ID NO: 10, and the second moiety comprising the sequence of SEQ ID NO: 9.

In some embodiments, the polypeptide comprises, from the N-terminus to the C-terminus, the third moiety comprising the Fc domain having the amino acid modifications of a)T307Q, Q311V and A378V; b) F234V, L235E and D265G; and c) S228P, the first moiety comprising the sequence of SEQ ID NO: 10, the second moiety comprising the sequence of SEQ ID NO: 15.

In some embodiments, the polypeptide comprises, from the N-terminus to the C-terminus, the third moiety comprising the Fc domain having the amino acid modifications of a) F234V, L235E and D265G; and b) S228P, the first moiety comprising the sequence of SEQ ID NO: 10, the second moiety comprising the sequence of SEQ ID NO: 9.

In some embodiments, the polypeptide comprises, from the N-terminus to the C-terminus, the third moiety comprising the Fc domain having the amino acid modification of S228P, the first moiety comprising the sequence of SEQ ID NO: 13, and the second moiety comprising the sequence of SEQ ID NO: 9.

In some embodiments, the polypeptide comprises, from the N-terminus to the C-terminus, the third moiety comprising the Fc domain having the amino acid modifications of a) F234V, L235E and D265G and b) S228P, the first moiety comprising the sequence of SEQ ID NO: 13, and the second moiety comprising the sequence of SEQ ID NO: 9.

In some embodiments, the polypeptide comprises, from the N-terminus to the C-terminus, the third moiety comprising the Fc domain having the amino acid modifications of a)T307Q, Q311V and A378V; b) F234V, L235E and D265G; and c) S228P, the first moiety comprising the sequence of SEQ ID NO: 13, and the second moiety comprising the sequence of SEQ ID NO: 9.

In some embodiments, the polypeptide comprises, from the N-terminus to the C-terminus, the third moiety comprising the Fc domain having the amino acid modification of S228P, the first moiety comprising the sequence of SEQ ID NO: 10, and the second moiety comprising the sequence of SEQ ID NO: 9.

In some embodiments, the polypeptide comprises, from the N-terminus to the C-terminus, the third moiety comprising the Fc domain having the amino acid modifications of a) F234V, L235E and D265G and b) S228P, the first moiety comprising the sequence of SEQ ID NO: 55, and the second moiety comprising the sequence of SEQ ID NO: 9.

In some embodiments, the C-terminus of the second moiety is linked to the N-terminus of the third moiety. In some embodiments, the N-terminus of the second moiety is linked to the C-terminus of the third moiety. As non-limiting examples, the polypeptide comprises from the N-terminus to the C-terminus, the first moiety, the second moiety and the Fc domain.

In some embodiments, the polypeptide comprises, from the N-terminus to the C-terminus, the first moiety comprising the sequence of SEQ ID NO: 13, the second moiety comprising the sequence of SEQ ID NO: 9 and the third moiety comprising the Fc domain having the amino acid modifications of a)T307Q, Q311V and A378V; b) F234V, L235E and D265G; and c) S228P.

In some embodiments, the polypeptide comprises, from the N-terminus to the C-terminus, the first moiety comprising the sequence of SEQ ID NO: 13, the second moiety comprising the sequence of SEQ ID NO: 9 and the third moiety comprising the Fc domain having the amino acid modification of S228P.

In some embodiments, the polypeptide comprises, from the N-terminus to the C-terminus, the first moiety comprising the sequence of SEQ ID NO: 10, the second moiety comprising the sequence of SEQ ID NO: 9 and the third moiety comprising the Fc domain having the amino acid modifications a)T307Q, Q311V and A378V; b) F234V, L235E and D265G; and c) S228P.

In some embodiments, the first moiety and the third moiety are linked by Linker 2. In some embodiments, Linker 2 is 5-10 amino acids or less. In some embodiments, Linker 2 is 1-5 amino acids.

In some embodiments, the first moiety and the third moiety are linked by a linker (Linker 3). In some embodiments, Linker 3 is 5-10 amino acids or less. In some embodiments, Linker 3 is 1-5 amino acids.

In some embodiments, Linker 2 comprises GGGGGS (SEQ ID NO: 41). In some embodiments, Linker 2 is GGGGGS (SEQ ID NO: 41).

In some embodiments, Linker 3 comprises GGGGGS (SEQ ID NO: 41). In some embodiments, Linker 3 is GGGGGS (SEQ ID NO: 41).

In some embodiments, the sequence of the polypeptide is SEQ ID NO: 3, 4, 5, 6, 7, 8 or 43-54. In some embodiments, the polypeptide comprises an amino acid of SEQ ID NO: 3. In some embodiments, the polypeptide comprises an amino acid of SEQ ID NO: 4. In some embodiments, the polypeptide comprises an amino acid of SEQ ID NO: 5. In some embodiments, the polypeptide comprises an amino acid of SEQ ID NO: 6. In some embodiments, the polypeptide comprises an amino acid of SEQ ID NO: 7. In some embodiments, the polypeptide comprises an amino acid of SEQ ID NO: 8. In some embodiments, the polypeptide comprises an amino acid of SEQ ID NO: 43. In some embodiments, the polypeptide comprises an amino acid of SEQ ID NO:44. In some embodiments, the polypeptide comprises an amino acid of SEQ ID NO:45. In some embodiments, the polypeptide comprises an amino acid of SEQ ID NO:46. In some embodiments, the polypeptide comprises an amino acid of SEQ ID NO:47. In some embodiments, the polypeptide comprises an amino acid of SEQ ID NO:48. In some embodiments, the polypeptide comprises an amino acid of SEQ ID NO:49. In some embodiments, the polypeptide comprises an amino acid of SEQ ID NO:50. In some embodiments, the polypeptide comprises an amino acid of SEQ ID NO:51. In some embodiments, the polypeptide comprises an amino acid of SEQ ID NO:52. In some embodiments, the polypeptide comprises an amino acid of SEQ ID NO:53. In some embodiments, the polypeptide comprises an amino acid of SEQ ID NO:54.

In some embodiments, the variable domain of the first moiety of said each polypeptide comprises HCDR1, HCDR2 and HCDR3, wherein the HCDR1, HCDR2 and HCDR3 comprises a) the sequence of SEQ ID NO: 16, 17, and 18, respectively; or b) the sequences of SEQ ID NO: 16, 26, and 22, respectively.

In some embodiments, the second moiety of said each polypeptide comprises a sequence having at least 85% sequence identity to the sequence of SEQ ID NO: 9. In some embodiments, the second moiety of said each polypeptide comprises a sequence having at least 90% sequence identity to the sequence of SEQ ID NO: 9. In some embodiments, the second moiety of said each polypeptide comprises a sequence having at least 92% sequence identity to the sequence of SEQ ID NO: 9. In some embodiments, the second moiety of said each polypeptide comprises a sequence having at least 95% sequence identity to the sequence of SEQ ID NO: 9. In some embodiments, the second moiety of said each polypeptide comprises a sequence having at least 97% sequence identity to the sequence of SEQ ID NO: 9. In some embodiments, the second moiety of said each polypeptide comprises a sequence having at least 98% sequence identity to the sequence of SEQ ID NO: 9. In some embodiments, the second moiety of said each polypeptide comprises a sequence having at least 99% sequence identity to the sequence of SEQ ID NO: 9. In some embodiments, the second moiety of said each polypeptide comprises a sequence of SEQ ID NO: 9.

In some embodiments, the second moiety of said each polypeptide comprises a sequence having at least 85% sequence identity to the sequence of SEQ ID NO: 14. In some embodiments, the second moiety of said each polypeptide comprises a sequence having at least 90% sequence identity to the sequence of SEQ ID NO: 14. In some embodiments, the second moiety of said each polypeptide comprises a sequence having at least 92% sequence identity to the sequence of SEQ ID NO: 14. In some embodiments, the second moiety of said each polypeptide comprises a sequence having at least 95% sequence identity to the sequence of SEQ ID NO: 14. In some embodiments, the second moiety of said each polypeptide comprises a sequence having at least 97% sequence identity to the sequence of SEQ ID NO: 14. In some embodiments, the second moiety of said each polypeptide comprises a sequence having at least 98% sequence identity to the sequence of SEQ ID NO: 14. In some embodiments, the second moiety of said each polypeptide comprises a sequence having at least 99% sequence identity to the sequence of SEQ ID NO: 14. In some embodiments, the second moiety of said each polypeptide comprises a sequence of SEQ ID NO: 14.

In some embodiments, the second moiety of said each polypeptide comprises a sequence having at least 85% sequence identity to the sequence of SEQ ID NO: 15. In some embodiments, the second moiety of said each polypeptide comprises a sequence having at least 90% sequence identity to the sequence of SEQ ID NO: 15. In some embodiments, the second moiety of said each polypeptide comprises a sequence having at least 92% sequence identity to the sequence of SEQ ID NO: 15. In some embodiments, the second moiety of said each polypeptide comprises a sequence having at least 95% sequence identity to the sequence of SEQ ID NO: 15. In some embodiments, the second moiety of said each polypeptide comprises a sequence having at least 97% sequence identity to the sequence of SEQ ID NO: 15. In some embodiments, the second moiety of said each polypeptide comprises a sequence having at least 98% sequence identity to the sequence of SEQ ID NO: 15. In some embodiments, the second moiety of said each polypeptide comprises a sequence having at least 99% sequence identity to the sequence of SEQ ID NO: 15. In some embodiments, the second moiety of said each polypeptide comprises a sequence of SEQ ID NO: 15.

In some embodiments, the Fc domain of said each polypeptide comprises one or more amino acid modifications of a) T307Q, Q311V and A378V; b) F234V, L235E and D265G; and c) S228P.

In some embodiments, the polypeptides of the present invention form a homodimer. In some embodiments, the polypeptides of the present invention form a heterodimer.

In some embodiments, the present invention provides a fusion protein comprising two identical polypeptides of the present invention.

In some embodiments, the polypeptide is encoded by nucleic acids. In some embodiments, the protein is encoded by nucleic acids.

In some embodiments, a vector comprises the nucleic acids encoding the polypeptide. In some embodiments, a vector comprises the nucleic acids encoding the protein.

In some embodiments, the vector is a viral vector.

In some embodiments, the viral vector is an adeno-associated virus (AAV) vector.

In some embodiments, the AAV vector is selected from AAV1, AAV2, AAV3, AAV4, AAV5, AAV6, AAV7, AAV8, AAV9, AAV10, AAV10, AAV11, AAV12, AAV13. In some embodiments, the AAV vector is AAV1. In some embodiments, the AAV vector is AAV2. In some embodiments, the AAV vector is AAV3. In some embodiments, the AAV vector is AAV4. In some embodiments, the AAV vector is AAV5. In some embodiments, the AAV vector is AAV6. In some embodiments, the AAV vector is AAV7. In some embodiments, the AAV vector is AAV8. In some embodiments, the AAV vector is AAV9. In some embodiments, the AAV vector is AAV10. In some embodiments, the AAV vector is AAV11. In some embodiments, the AAV vector is AAV12. In some embodiments, the AAV vector is AAV13.

In some embodiments, a liquid nanoparticle (LNP) comprises a nucleic acid or the vector.

In some embodiments, a host cell comprises a nucleic acid or the vector. In some embodiments, a host cell comprises a nucleic acid. In some embodiments, a host cell comprises a vector.

In one aspect, the present invention provides, among other things, a method of producing a polypeptide or a protein comprising culturing a host cell under conditions that allow production of the polypeptide or the protein.

In some embodiments, the method further comprising isolating the polypeptide or the protein.

In one aspect, the present invention provides, among other things, a pharmaceutical composition comprising a polypeptide of the present invention and one or more physiologically acceptable carriers, excipients, or diluents.

In one aspect, the present invention provides, among other things, a pharmaceutical composition comprising a protein of the present invention and one or more physiologically acceptable carriers, excipients, or diluents.

In one aspect, the present invention provides, among other things, a method of treating a disease or disorder associated with C3b in a subject in need thereof, comprising administering to the subject the polypeptide or the protein of the present invention.

In one aspect, the present invention provides, among other things, a method of treating a disease or disorder associated with C3b in a subject in need thereof, comprising administering to the subject a nucleic acid encoding a polypeptide or the protein of the present invention.

In some embodiments, the disease or disorder is dense deposit disease (DDD). In some embodiments, the disease or disorder is C3 glomerulonephritis. In some embodiments, the disease or disorder is passive heymann nephritis. In some embodiments, the disease or disorder is age-related macular degeneration.

In one aspect, the present invention provides, among other things, a method of modulating alternative complement activity in a subject comprising administering to the subject a nucleic acid encoding a polypeptide or the protein of the present invention.

In some embodiments, the method inhibits C3 convertase.

### BRIEF DESCRIPTION OF THE DRAWINGS

The drawings are for illustration purposes only, not for limitation.

FIG. 1A is an exemplary graph showing inhibition of Factor B binding to C3b as determined by ELISA. FIG. 1B is an exemplary SDS-PAGE gel showing C3b→iC3b and fB→Bb conversion of alternative pathway after treatment with test articles.

FIG. 2 shows cryo-EM structures of exemplary C3b inhibitor polypeptides in complex with C3b.

FIG. 3A is a series of exemplary graphs showing C3 deposition in 50% normal human serum (NHS), normal cynomolgus monkey serum (NCS), or individual human donor serum (KP68861 and KP68862) as determined by ELISA. FIG. 3B is a series of exemplary graphs showing alternative pathway (AP) inhibition as indicated by MAC deposition using 6% normal human serum (NHS) and 6% normal cynomolgus monkey serum (NCS). FIG. 3C is a series of exemplary graphs showing C3 consumption in normal cynomolgus monkey serum (NCS).

FIG. 4A is a series of exemplary graphs showing the effect of test antibodies on the classical C3 pathway as determined by measuring the deposit of the C3b on the surface of an IgM-coated plate in factor B-depleted serum or C5-depleted serum as well as a graph showing the effects of C3b inhibition on membrane attack complex (MAC) formation. FIG. 4B is a series of exemplary graphs showing the effects of test antibodies on either the alternative pathway or the classical pathway.

FIG. 5A shows a measurement of alternative pathway inhibition by quantification of a hemolytic assay in the serum of cynomolgus monkeys. FIG. 5B shows a measurement of alternative pathway inhibition by quantification of a hemolytic assay in the serum of humans. FIG. 5C shows a measurement of either total hemolysis or alternative pathway-specific hemolysis in the serum of humans.

FIG. 6A shows the results of C3b inhibition on C3 area in a mouse model with hyperactive alternative pathway using two different administration strategies. FIG. 6B shows cellular staining and quantifies C3 deposits following treatment with C3b inhibitors.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Any numerical values used in this application are meant to cover any variations within the standard deviation or normal fluctuations appreciated by one of ordinary skill in the relevant art.

Antibody: As used herein, the term “antibody” refers to immunoglobulin molecules and immunologically active portions of immunoglobulin (Ig) molecules, i.e., molecules that contain an antigen binding site that binds (immunoreacts with) an antigen. By “binds” or “immunoreacts with” is meant that the antibody reacts with one or more antigenic determinants of the desired. Antibodies include antibody fragments. Antibodies also include, but are not limited to, polyclonal, monoclonal, chimeric dAb (domain antibody), single chain, Fab, Fab′, F(ab′)2 fragments, scFvs, single-domain antibodies (sdAb), and VHHs. An antibody may be a whole antibody, or immunoglobulin, or an antibody fragment.

Antibody dependent cytotoxicity: As used herein, the term “antibody-dependent cytotoxicity” or “ADCC” refers to lysis of human target cells by an antibody according to the invention in the presence of effector cells.

Fab arm exchange: The term, “Fab arm exchange” refers to the phenomenon that IgG4 antibodies can exchange ‘half-molecules’, an activity termed Fab arm exchange herein. Especially in bispecific or biparatopic molecules, this results in functionally monovalent antibodies with unknown specificity and hence, potentially, reduced therapeutic efficacy. Mutations can be introduced in the Fc region to inhibit the Fab arm exchange. It is known that S228P mutation can prevent IgG4 FAE to undetectable levels both in vitro and in vivo.

Fc region: As used herein, the term “Fc region” refers to a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

IC₅₀: As used herein, the term “IC₅₀” refers to the concentration needed to inhibit half of the maximum biological response of the ligand agonist, and is generally determined by competition binding assays.

Linker: As used herein, the term “linker” refers to a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-terminus of proteins or protein domains, linkage via disulfide bonds, and linkage via chemical cross-linking reagents. In one aspect of this embodiment, the linker is a peptide bond, generated by recombinant techniques or peptide synthesis. In some embodiments, the linker may contain amino acid residues that provide flexibility. Thus, the linker peptide may predominantly include the following amino acid residues: Gly (G), Ser (S), Ala (A), or Thr (T). The linker peptide should have a length that is adequate to link two molecules in such a way that they assume the correct conformation relative to one another so that they retain the desired activity.

Identity: As used herein, the term “identity” refers a relationship between the sequences of two or more polypeptide molecules or two or more nucleic acid molecules as known in the art, comparing the sequences of these molecules. In the art, “identity” also means the degree of sequence relatedness between nucleic acid molecules or polypeptides, and in some cases more than one nucleotide sequence or more than one. It may be determined by a match between amino acid sequence strings. “Identity” means between a gap alignment (if any) addressed by a particular mathematical model or computer program (ie, an “algorithm”) and a smaller sequence of two or more sequences.

Inhibition: the terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater.

Vector: The term “vector” refers to a polynucleotide (usually DNA) used to artificially carry foreign genetic material to another cell where it can be replicated or expressed. Non-limiting exemplary vectors include plasmids, viral vectors, cosmids, and artificial chromosomes. Such vectors may be derived from a variety of sources, including bacterial and viral sources. A non-limiting exemplary viral source for a plasmid is adeno-associated virus.

### DETAILED DESCRIPTION

The present invention provides, among other things, C3b inhibitor polypeptides comprising a variable domain capable of binding to C3b and an amino acid sequence of membrane cofactor protein (MCP) and therapeutic proteins containing the same. In particular, the polypeptides and therapeutic proteins of the present invention selectively bind to C3b with high affinity, but do not bind to C3, and inhibit alternative pathway of complement system without affecting the initiation of classical and lectin pathways. Without wishing to be bound by any particular theory, the present invention encompasses C3b inhibitor polypeptides and therapeutic proteins thereof that inhibit alternative pathway specifically and those that inhibit the downstream components and steps shared by both alternative pathway and classic pathway through, for example, inhibition of C3b, the central and activated component of alternative, classic and lectin pathways.

The present invention further provides uses of the polypeptides, proteins and compositions disclosed herein. In some embodiments, the present invention provides methods of treating complement associated diseases such as, but not limited to: C3 glomerulopathy (C3G), including C3 Glomerulonephritis (C3GN) Dense Deposit Disease (DDD), Paroxysmal Nocturnal Hemoglobinuria, Geographic Atrophy (GA), Age-related Macular Degeneration ((w)AMD), hematuria, proteinuria, Acute Kidney Injury (AKI), Kidney Disease (CKD), autoimmune hemolytic anemia (AIHA), rheumatoid arthritis (RA), Membranous Nephropathy (MN), Diabetic Kidney Disease (DKD), and Sepsis. In some embodiments, the C3b targeted polypeptides can be used for the treatment of other disorders caused by excessively activated and dysregulated complement activation such as Chronic antibody mediated rejection (cAMR), chronic inflammatory demyelinating polyneuropathy (CIDP), Dermatomyositis (DM). Complement System

The complement system is a part of the innate immune system which does not adapt to changes over the course of the host's life, but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells) and agglutination (clustering and binding of pathogens together). The complement system has three pathways: classical, alternative and lectin. Classical pathway (CP) is activated by certain isotypes of antibodies bound to antigens. Alternative pathway (AP) is activated on microbial cell surfaces in the absence of antibody. Lectin pathway is activated by a plasma lectin that binds to mannose residues on microbes. Although the pathways of complement activation differ in how they are initiated, all of them result in the generation of enzyme complexes that are able to cleave the most abundant complement protein, C3.

### Classical Pathway (CP)

The classical pathway, so called because it was discovered first, uses a plasma protein called C1q to detect antibodies bound to the surface of a microbe or other structure. Once C1q binds to the Fc portion of the antibodies, two associated serine proteases, called C1r and C1s, become active and initiate a proteolytic cascade involving other complement proteins. The classical pathway is one of the major effector mechanisms of the humoral arm of adaptive immune responses. Innate immune system soluble proteins called pentraxins, can also bind C1q and initiate the classical pathway.

### Lectin Pathway

Lectin pathway is triggered by a plasma protein called mannose-binding lectin (MBL), which recognizes terminal mannose residues on microbial glycoproteins and glycolipids, similar to the mannose receptor on phagocyte membranes described earlier. MBL is a member of the collection family with a hexameric structure similar to the C1q component of the complement system. After MBL binds to microbes, two zymogens called MASP1 (mannose-associated serine protease 1, or mannan-binding lectin-associated serine protease) and MASP2, with similar functions to C1r and C1s, associate with MBL and initiate downstream proteolytic steps identical to the classical pathway. The central event in complement activation is proteolysis of the complement protein C3 to generate biologically active products and the subsequent covalent attachment of a product of C3, called C3b, to microbial cell surfaces or to antibody bound to antigen. Complement activation depends on the generation of two proteolytic complexes: the C3 convertase, which cleaves C3 into two proteolytic fragments called C3a and C3b; and the C5 convertase, which cleaves C5 into C5a and C5b.

### Alternative Pathway (AP)

The third pathway of complement activation is called the alternative pathway because it was discovered as a second, or ‘alternative,’ pathway for complement activation after the classical pathway had been defined. This pathway can proceed on many microbial surfaces in the absence of specific antibody, and it leads to the generation of a distinct C3 convertase designated C3bBb. In contrast to the classical and MB-lectin pathways of complement activation, the alternative pathway does not depend on a pathogen-binding protein for its initiation; instead, it is initiated through the spontaneous hydrolysis of C3. A number of mechanisms ensure that the activation pathway will only proceed on the surface of a pathogen.

### C3 Convertase and C3b

C3 is abundant in plasma, and C3b is produced at a significant rate by spontaneous cleavage (also known as ‘tickover’). This occurs through the spontaneous hydrolysis of the thioester bond in C3 to form C3(H2O) which has an altered conformation, allowing binding of the plasma protein factor B. The binding of B by C3(H2O) then allows a plasma protease called factor D to cleave factor B to Ba and Bb, the latter remaining associated with C3(H2O) to form the C3(H2O)Bb complex. This complex is a fluid-phase C3 convertase, and although it is only formed in small amounts it can cleave many molecules of C3 to C3a and C3b. Much of this C3b is inactivated by enzymatic cleavage into iC3b, but some attaches covalently, through its reactive thioester group, to the surfaces of host cells or to pathogens. C3b bound in this way is able to bind factor B, allowing its cleavage by factor D to yield the small fragment Ba and the active protease Bb. This results in formation of the alternative pathway C3 convertase, C3b, Bb.

When C3b binds to host cells, a number of complement-regulatory proteins, present in the plasma and on host cell membranes combine to prevent complement activation from proceeding. These proteins interact with C3b and either prevent the convertase from forming, or promote its rapid dissociation. Thus, the complement receptor 1 (CR1) and a membrane-attached protein known as decay-accelerating factor (DAF or CD55) compete with factor B for binding to C3b on the cell surface, and can displace Bb from a convertase that has already formed. Convertase formation can also be prevented by cleaving C3b to its inactive derivative iC3b. This is achieved by a plasma protease, factor I, in conjunction with C3b-binding proteins that can act as cofactors, such as CR1 and membrane cofactor of proteolysis (MCP or CD46), another host cell membrane protein. Factor H is another complement-regulatory protein in plasma that binds C3b and, like CR1, it is able to compete with factor B and displace Bb from the convertase in addition to acting as a cofactor for factor I. Factor H binds preferentially to C3b bound to vertebrate cells as it has an affinity for the sialic acid residues present on these cells.

By contrast, because pathogen surfaces lack these regulatory proteins and sialic acid residues, the C3bBb convertase can form and persist. Indeed, this process may be favored by a positive regulatory factor, known as properdin or factor P, which binds to many microbial surfaces and stabilizes the convertase. Deficiencies in factor P are associated with a heightened susceptibility to infection with Neisseria species. Once formed, the C3b, Bb convertase rapidly cleaves yet more C3 to C3b, which can bind to the pathogen and either act as an opsonin or reinitiate the pathway to form another molecule of C3bBb convertase. Thus, the alternative pathway activates through an amplification loop that can proceed on the surface of a pathogen, but not on a host cell. This same amplification loop enables the alternative pathway to contribute to complement activation initially triggered through the classical or MB-lectin pathways.

The C3 convertases resulting from activation of the classical and MB-lectin pathways (C4b, 2b) and from the alternative pathway (C3b, Bb) are apparently distinct. Only one component of the alternative pathway appears entirely unrelated to its functional equivalents in the classical and MB-lectin pathways; this is the initiating serine protease, factor D. Factor D can also be singled out as the only activating protease of the complement system to circulate as an active enzyme rather than a zymogen. This is both necessary for the initiation of the alternative pathway through spontaneous C3 cleavage, and safe for the host because factor D has no other substrate than factor B when bound to C3b. This means that factor D only finds its substrate at a very low level in plasma, and at pathogen surfaces where the alternative pathway of complement activation is allowed to proceed.

The formation of C3 convertases is the point at which the three pathways of complement activation converge, because both the classical pathway and MB-lectin pathway convertases C4b,2b, and the alternative pathway convertase C3bBb have the same activity, and they initiate the same subsequent events. They both cleave C3 to C3b and C3a. C3b binds covalently through its thioester bond to adjacent molecules on the pathogen surface; otherwise it is inactivated by hydrolysis. C3 is the most abundant complement protein in plasma, occurring at a concentration of 1.2 mg ml, and up to 1000 molecules of C3b can bind in the vicinity of a single active C3 convertase. Thus, the main effect of complement activation is to deposit large quantities of C3b on the surface of the infecting pathogen, where it forms a covalently bonded coat that can signal the ultimate destruction of the pathogen by phagocytes.

The next step in the cascade is the generation of the C5 convertases. In the classical and the MB-lectin pathways, a C5 convertase is formed by the binding of C3b to C4b,2b to yield C4b,2b,3b. By the same token, the C5 convertase of the alternative pathway is formed by the binding of C3b to the C3 convertase to form C3b2Bb. C5 is captured by these C5 convertase complexes through binding to an acceptor site on C3b, and is then rendered susceptible to cleavage by the serine protease activity of C2b or Bb. This reaction, which generates C5b and C5a, is much more limited than cleavage of C3, as C5 can be cleaved only when it binds to C3b that is part of the C5 convertase complex. Thus, complement activation by both the alternative, MB-lectin and classical pathways leads to the binding of large numbers of C3b molecules on the surface of the pathogen, the generation of a more limited number of C5b molecules, and the release of C3a and C5a.

C3b is the central and converged point for Classic, Alternative or lectin complement pathways, inactivating C3b could potentially block all three pathways simultaneously and/or retain the functions of other complement factors such as those involved in the initiation of Classic pathway. A C3b targeted inhibitor, for example, the polypeptides and therapeutic proteins of the present invention could inhibit inactivate alternative pathways. Some can also block for example the downstream components and steps that are shared by alternation pathway and classic pathway.

### C3b Inhibitor Polypeptides and Proteins

In one aspect, the present invention provides, among other things, a polypeptide that is capable of binding to C3b, wherein the polypeptide comprises a first moiety and a second moiety, wherein the first moiety comprises a variable domain capable of binding to C3b, and the second moiety comprises an amino acid sequence of membrane cofactor protein (MCP) or a variant thereof.

In one aspect, the present invention provides, among other things, a protein comprising two polypeptides, wherein each polypeptide is capable of binding to complement C3b (C3b) and comprises a first moiety and a second moiety, wherein the first moiety comprises a variable domain capable of binding to C3b, and the second moiety comprises an amino acid sequence of membrane cofactor protein (MCP) or a variant thereof. In some embodiments, the protein is a homodimer, comprising two identical polypeptides described herein. In other embodiments, the protein is heterodimer.

In some embodiments, each polypeptide binds to C3b, but not to C3.

In some embodiments, the first moiety and second moiety are linked. In some embodiments, the first moiety and second moiety are linked via a peptide linker.

In some embodiments, the polypeptide comprises from the N-terminus to the C-terminus, the first moiety linked to the second moiety. In alternative embodiments, the polypeptide comprises from the N-terminus to the C-terminus, the second moiety linked to the first moiety.

In some embodiments, the polypeptide described herein further comprises a third moiety which is a half-life extension moiety. In some embodiments, the third moiety is a Fc domain or variant thereof.

In other non-limiting examples, the polypeptide comprises a first moiety comprising an antibody or an antigen binding domain thereof that binds to C3b, a second moiety comprising an amino acid sequence of MCP or a variant thereof, and a third moiety which is a Fc domain.

In some embodiments, the polypeptide described herein comprises from the N-terminus to the C-terminus, the third moiety, the first moiety and the second moiety. In some embodiments, the polypeptide described herein comprises from the N-terminus to the C-terminus, the first moiety, the second moiety and the third moiety. In some embodiments, the polypeptide described herein comprises from the N-terminus to the C-terminus, the second moiety, the first moiety and the third moiety.

In some embodiments, the polypeptide described herein forms a dimer; the dimer can be a homodimer comprising two identical polypeptides, or a heterodimer comprising two non-identical polypeptides, e.g., two polypeptides with different first moieties, second moieties or Fc domains.

Accordingly, the polypeptides or the proteins described herein can inhibit formation of C3 convertase and provides bifunctional inhibition of complement Alternative pathway (AP). In some embodiments, the polypeptides and proteins of the present invention selectively bind to C3b and inhibit the C3 convertase formation and mediate clearance of C3 deposits. In some embodiments, the polypeptides and proteins of the present invention can inhibit the downstream components that are shared by all complement systems.

In some embodiments, the polypeptide described herein is a biparatopic polypeptide.

In some embodiments, the biparatopic polypeptide comprises the first moiety and the second moiety described herein, wherein the first moiety and the second moiety each binds to a single molecule of C3b at the same time.

In some embodiments, the biparatopic polypeptide comprises the first moiety and the second moiety described herein, wherein the first moiety and the second moiety each binds to a single molecule of C3(H2O) at the same time.

In some embodiments, the polypeptide described herein is a bifunctional polypeptide. In some embodiments, the bifunctional polypeptide comprises the first moiety and the second moiety described herein, wherein the first moiety i) prevents the association of factor B with C3b or C3(H2O) and/or ii) hinders formation of C3 convertase, wherein the second moiety iii) recruits factor I to C3b or C3(H2O); and/or iv) facilitates proteolytic cleavage of C3b or C3(H2O).

In some embodiments, the polypeptide described herein is a biparatopic and bifunctional polypeptide described herein.

### Variable Domain Targeting C3b

In some embodiments, the first moiety binds to C3b. In some embodiments, the first moiety binds to C3(H2O). In some embodiments, the first moiety does not bind to C3. In some embodiments, the first moiety has reduced affinity to C3 compared to C3b. In some embodiments, the first moiety has reduced affinity to C3 compared to C3(H2O). In some embodiments, the first moiety has reduced affinity to C3 compared to C3b and C3(H2O).

In some embodiments, the variable domain of the first moiety binds to C3(H2O). In some embodiments, the variable domain of the first moiety does not bind to C3. In some embodiments, the variable domain of the first moiety has reduced affinity to C3 compared to C3b. In some embodiments, the variable domain of the first moiety has reduced affinity to C3 compared to C3(H2O). In some embodiments, the variable domain of the first moiety has reduced affinity to C3 compared to C3b and C3(H2O).

In some embodiments, the first moiety of the polypeptide comprises a variable domain capable of binding to C3b. In some embodiments, the first moiety comprises an antibody or an antibody fragment capable of binding to C3b.

A traditional antibody, also known as an immunoglobulin, is a Y-shaped structure which consists of four polypeptides—two heavy chains and two light chains. This structure allows antibody molecules to carry out their dual functions: antigen binding and biological activity mediation. Each function is carried out by different parts of the antibody: fragment antigen-binding (Fab fragment) and fragment crystallizable region (Fc region). Fab fragment is a region on an antibody that binds to antigens. It is composed of one constant and one variable domain of each of the heavy and the light chain. These domains shape the paratope—the antigen-binding site—at the amino terminal end of the monomer. Fc region is the tail region of an antibody that interacts with cell surface receptors called Fc receptors and some proteins of the complement system. This property allows antibodies to activate the immune system. The Fc regions of immunoglobulin Gs bear a highly conserved N-glycosylation site.

Within the VH and VL regions exists three CDRs, hypervariable amino acid sequences, forming the antigen-binding sites of each Fab. The interface of each heavy and light chain CDRs with the constant segments creates a three-dimensional binding structure with high specificity for a distinct antigenic determinant or epitope.

In some embodiments, the first moiety comprises a Fab. In some embodiments, the first moiety comprises a F(ab′)2. In some embodiments, the first moiety comprises a scFv. In some embodiments, the first moiety comprises a VHH, also known as sdAb and nanobody.

In some embodiments, the antibody or the antibody fragment thereof is a single domain antibody (VHH). A single domain antibody (VHH) is a fragment represents the smallest antigen binding domains of antibodies. Despite having a molecular weight typically less than its parent antibody of 12-15 KDa, this type of antibody retains its binding specificity and affinity. This type of single-domain antibody is characterized by its high physical and thermal stability and production process.

In some embodiments, the variable domain of the first moiety is a VHH.

In some embodiments, the variable domain of the first moiety is a single domain antibody.

In some embodiments, the variable domain of the first moiety comprises HCDR1. In some embodiments, the variable domain of the first moiety comprises HCDR2. In some embodiments, the variable domain of the first moiety comprises HCDR3. In some embodiments, the variable domain of the first moiety comprises HCDR1, HCDR2, and HCDR3. The terms CDR1, CDR2, and CDR3 and the terms HCDR1, HCDR2, and HCDR3, respectively, are used interchangeably.

In some embodiments, the variable domain of the first moiety comprises CDRs: CDR1, CDR2, and CDR3 as identified by the amino acid sequences in Table A and Table B.

Exemplary anti-C3b antibody sequences are provided below:

TABLE A
CDR Sequences of Exemplary
Anti-C3bVHHs based on Kabat convention

| | CDRs | | | | | |
|---|---|---|---|---|---|---|
| | | SEQ | | SEQ | | SEQ |
| | | ID | | ID | | ID |
| Ab | CDR1 | NO | CDR2 | NO | CDR3 | NO |
| APAb1 | NYHMG | 16 | TIIRTGTTI | 24 | ATSGWHIK | 22 |
| | | | YYADSVKG | | TVTPYEY | |
| APAb2 | NYHMG | 16 | TIIRHGTTI | 26 | ATSGWHIK | 22 |
| | | | YYADSVKG | | TVTPYEY | |
| APAb3 | NYHMG | 16 | TIIRTGETI | 17 | ATSGWNIK | 18 |
| | | | YYADSVKG | | TVTPYEY | |
| APAb4 | NYHMG | 16 | TIIRTGKTI | 21 | ATSGWHIK | 22 |
| | | | YYADSVKG | | TVTPYEY | |

TABLE B
CDR Sequences of Exemplary
Anti-C3b VHHs based on Chothia convention

| | CDRs | | | | | |
|---|---|---|---|---|---|---|
| | | SEQ | | SEQ | | SEQ |
| | | ID | | ID | | ID |
| Ab | CDR1 | NO | CDR2 | NO | CDR3 | NO |
| APAb1 | GRTFSNY | 19 | IRTGTT | 25 | ATSGWHIK | 22 |
| | | | | | TVTPYEY | |
| APAb2 | GRTFSNY | 19 | IRHGTT | 27 | ATSGWHIK | 22 |
| | | | | | TVTPYEY | |
| APAb3 | GRTFSNY | 19 | IRTGET | 20 | ATSGWNIK | 18 |
| | | | | | TVTPYEY | |
| APAb4 | GRTFSNY | 19 | IRTGKT | 23 | ATSGWHIK | 22 |
| | | | | | TVTPYEY | |

In some embodiments, the variable domain of the first moiety comprises CDR amino acid sequences with at least 75%, 78%, 80%, 82%, 85%, 87%, 90%, 91%, 92%, 93%, 94%, 95%, 96%, 97%, 98%, 99% or more identity with one or more of SEQ ID NO: 16-27.

In some embodiments, the variable domain of the first moiety comprises: a CDR1 amino acid sequence with at least 75% identity with SEQ ID NO: 16. In some embodiments, the variable domain of the first moiety comprises: a CDR1 amino acid sequence with at least 80% identity with SEQ ID NO: 16. In some embodiments, the variable domain of the first moiety comprises: a CDR1 amino acid sequence with at least 85% identity with SEQ ID NO: 16. In some embodiments, the variable domain of the first moiety comprises: a CDR1 amino acid sequence with at least 88% identity with SEQ ID NO: 16. In some embodiments, the variable domain of the first moiety comprises: a CDR1 amino acid sequence with at least 90% identity with SEQ ID NO: 16. In some embodiments, the variable domain of the first moiety comprises: a CDR1 amino acid sequence with at least 92% identity with SEQ ID NO: 16. In some embodiments, the variable domain of the first moiety comprises: a CDR1 amino acid sequence with at least 95% identity with SEQ ID NO: 16. In some embodiments, the variable domain of the first moiety comprises: a CDR1 amino acid sequence with at least 98% identity with SEQ ID NO: 16.

In some embodiments, the variable domain of the first moiety comprises: a CDR2 amino acid sequence with at least 75% identity with SEQ ID NO: 17. In some embodiments, the variable domain of the first moiety comprises: a CDR2 amino acid sequence with at least 80% identity with SEQ ID NO: 17. In some embodiments, the variable domain of the first moiety comprises: a CDR2 amino acid sequence with at least 85% identity with SEQ ID NO: 17. In some embodiments, the variable domain of the first moiety comprises: a CDR2 amino acid sequence with at least 88% identity with SEQ ID NO: 17. In some embodiments, the variable domain of the first moiety comprises: a CDR2 amino acid sequence with at least 90% identity with SEQ ID NO: 17. In some embodiments, the variable domain of the first moiety comprises: a CDR2 amino acid sequence with at least 92% identity with SEQ ID NO: 17. In some embodiments, the variable domain of the first moiety comprises: a CDR2 amino acid sequence with at least 95% identity with SEQ ID NO: 17. In some embodiments, the variable domain of the first moiety comprises: a CDR2 amino acid sequence with at least 98% identity with SEQ ID NO: 17.

In some embodiments, the variable domain of the first moiety comprises: a CDR3 amino acid sequence with at least 75% identity with SEQ ID NO: 18. In some embodiments, the variable domain of the first moiety comprises: a CDR3 amino acid sequence with at least 80% identity with SEQ ID NO: 18. In some embodiments, the variable domain of the first moiety comprises: a CDR3 amino acid sequence with at least 85% identity with SEQ ID NO: 18. In some embodiments, the variable domain of the first moiety comprises: a CDR3 amino acid sequence with at least 88% identity with SEQ ID NO: 18. In some embodiments, the variable domain of the first moiety comprises: a CDR3 amino acid sequence with at least 90% identity with SEQ ID NO: 18. In some embodiments, the variable domain of the first moiety comprises: a CDR3 amino acid sequence with at least 92% identity with SEQ ID NO: 18. In some embodiments, the variable domain of the first moiety comprises: a CDR3 amino acid sequence with at least 95% identity with SEQ ID NO: 18. In some embodiments, the variable domain of the first moiety comprises: a CDR3 amino acid sequence with at least 98% identity with SEQ ID NO: 18.

In some embodiments, the variable domain of the first moiety comprises: a CDR1 amino acid sequence with at least 75% identity with SEQ ID NO: 19. In some embodiments, the variable domain of the first moiety comprises: a CDR1 amino acid sequence with at least 80% identity with SEQ ID NO: 19. In some embodiments, the variable domain of the first moiety comprises: a CDR1 amino acid sequence with at least 85% identity with SEQ ID NO: 19. In some embodiments, the variable domain of the first moiety comprises: a CDR1 amino acid sequence with at least 88% identity with SEQ ID NO: 19. In some embodiments, the variable domain of the first moiety comprises: a CDR1 amino acid sequence with at least 90% identity with SEQ ID NO: 19. In some embodiments, the variable domain of the first moiety comprises: a CDR1 amino acid sequence with at least 92% identity with SEQ ID NO: 19. In some embodiments, the variable domain of the first moiety comprises: a CDR1 amino acid sequence with at least 95% identity with SEQ ID NO: 19. In some embodiments, the variable domain of the first moiety comprises: a CDR1 amino acid sequence with at least 98% identity with SEQ ID NO: 19.

In some embodiments, the variable domain of the first moiety comprises: a CDR2 amino acid sequence with at least 75% identity with SEQ ID NO: 20. In some embodiments, the variable domain of the first moiety comprises: a CDR2 amino acid sequence with at least 80% identity with SEQ ID NO: 20. In some embodiments, the variable domain of the first moiety comprises: a CDR2 amino acid sequence with at least 85% identity with SEQ ID NO: 20. In some embodiments, the variable domain of the first moiety comprises: a CDR2 amino acid sequence with at least 88% identity with SEQ ID NO: 20. In some embodiments, the variable domain of the first moiety comprises: a CDR2 amino acid sequence with at least 90% identity with SEQ ID NO: 20. In some embodiments, the variable domain of the first moiety comprises: a CDR2 amino acid sequence with at least 92% identity with SEQ ID NO: 20. In some embodiments, the variable domain of the first moiety comprises: a CDR2 amino acid sequence with at least 95% identity with SEQ ID NO: 20. In some embodiments, the variable domain of the first moiety comprises: a CDR2 amino acid sequence with at least 98% identity with SEQ ID NO: 20.

In some embodiments, the variable domain of the first moiety comprises: a CDR2 amino acid sequence with at least 75% identity with SEQ ID NO: 21. In some embodiments, the variable domain of the first moiety comprises: a CDR2 amino acid sequence with at least 80% identity with SEQ ID NO: 21. In some embodiments, the variable domain of the first moiety comprises: a CDR2 amino acid sequence with at least 85% identity with SEQ ID NO: 21. In some embodiments, the variable domain of the first moiety comprises: a CDR2 amino acid sequence with at least 88% identity with SEQ ID NO: 21. In some embodiments, the variable domain of the first moiety comprises: a CDR2 amino acid sequence with at least 90% identity with SEQ ID NO: 21. In some embodiments, the variable domain of the first moiety comprises: a CDR2 amino acid sequence with at least 92% identity with SEQ ID NO: 21. In some embodiments, the variable domain of the first moiety comprises: a CDR2 amino acid sequence with at least 95% identity with SEQ ID NO: 21. In some embodiments, the variable domain of the first moiety comprises: a CDR2 amino acid sequence with at least 98% identity with SEQ ID NO: 21.

In some embodiments, the variable domain of the first moiety comprises: a CDR3 amino acid sequence with at least 75% identity with SEQ ID NO: 22. In some embodiments, the variable domain of the first moiety comprises: a CDR3 amino acid sequence with at least 80% identity with SEQ ID NO: 22. In some embodiments, the variable domain of the first moiety comprises: a CDR3 amino acid sequence with at least 85% identity with SEQ ID NO: 22. In some embodiments, the variable domain of the first moiety comprises: a CDR3 amino acid sequence with at least 88% identity with SEQ ID NO: 22. In some embodiments, the variable domain of the first moiety comprises: a CDR3 amino acid sequence with at least 90% identity with SEQ ID NO: 22. In some embodiments, the variable domain of the first moiety comprises: a CDR3 amino acid sequence with at least 92% identity with SEQ ID NO: 22. In some embodiments, the variable domain of the first moiety comprises: a CDR3 amino acid sequence with at least 95% identity with SEQ ID NO: 22. In some embodiments, the variable domain of the first moiety comprises: a CDR3 amino acid sequence with at least 98% identity with SEQ ID NO: 22.

In some embodiments, the variable domain of the first moiety comprises: a CDR2 amino acid sequence with at least 75% identity with SEQ ID NO: 23. In some embodiments, the variable domain of the first moiety comprises: a CDR2 amino acid sequence with at least 80% identity with SEQ ID NO: 23. In some embodiments, the variable domain of the first moiety comprises: a CDR2 amino acid sequence with at least 85% identity with SEQ ID NO: 23. In some embodiments, the variable domain of the first moiety comprises: a CDR2 amino acid sequence with at least 88% identity with SEQ ID NO: 23. In some embodiments, the variable domain of the first moiety comprises: a CDR2 amino acid sequence with at least 90% identity with SEQ ID NO: 23. In some embodiments, the variable domain of the first moiety comprises: a CDR2 amino acid sequence with at least 92% identity with SEQ ID NO: 23. In some embodiments, the variable domain of the first moiety comprises: a CDR2 amino acid sequence with at least 95% identity with SEQ ID NO: 23. In some embodiments, the variable domain of the first moiety comprises: a CDR2 amino acid sequence with at least 98% identity with SEQ ID NO: 23.

In some embodiments, the variable domain of the first moiety comprises: a CDR2 amino acid sequence with at least 75% identity with SEQ ID NO: 24. In some embodiments, the variable domain of the first moiety comprises: a CDR2 amino acid sequence with at least 80% identity with SEQ ID NO: 24. In some embodiments, the variable domain of the first moiety comprises: a CDR2 amino acid sequence with at least 85% identity with SEQ ID NO: 24. In some embodiments, the variable domain of the first moiety comprises: a CDR2 amino acid sequence with at least 88% identity with SEQ ID NO: 24. In some embodiments, the variable domain of the first moiety comprises: a CDR2 amino acid sequence with at least 90% identity with SEQ ID NO: 24. In some embodiments, the variable domain of the first moiety comprises: a CDR2 amino acid sequence with at least 92% identity with SEQ ID NO: 24. In some embodiments, the variable domain of the first moiety comprises: a CDR2 amino acid sequence with at least 95% identity with SEQ ID NO: 24. In some embodiments, the variable domain of the first moiety comprises: a CDR2 amino acid sequence with at least 98% identity with SEQ ID NO: 24.

In some embodiments, the variable domain of the first moiety comprises: a CDR2 amino acid sequence with at least 75% identity with SEQ ID NO: 25. In some embodiments, the variable domain of the first moiety comprises: a CDR2 amino acid sequence with at least 80% identity with SEQ ID NO: 25. In some embodiments, the variable domain of the first moiety comprises: a CDR2 amino acid sequence with at least 85% identity with SEQ ID NO: 25. In some embodiments, the variable domain of the first moiety comprises: a CDR2 amino acid sequence with at least 88% identity with SEQ ID NO: 25. In some embodiments, the variable domain of the first moiety comprises: a CDR2 amino acid sequence with at least 90% identity with SEQ ID NO: 25. In some embodiments, the variable domain of the first moiety comprises: a CDR2 amino acid sequence with at least 92% identity with SEQ ID NO: 25. In some embodiments, the variable domain of the first moiety comprises: a CDR2 amino acid sequence with at least 95% identity with SEQ ID NO: 25. In some embodiments, the variable domain of the first moiety comprises: a CDR2 amino acid sequence with at least 98% identity with SEQ ID NO: 25.

In some embodiments, the variable domain of the first moiety comprises: a CDR2 amino acid sequence with at least 75% identity with SEQ ID NO: 26. In some embodiments, the variable domain of the first moiety comprises: a CDR2 amino acid sequence with at least 80% identity with SEQ ID NO: 26. In some embodiments, the variable domain of the first moiety comprises: a CDR2 amino acid sequence with at least 85% identity with SEQ ID NO: 26. In some embodiments, the variable domain of the first moiety comprises: a CDR2 amino acid sequence with at least 88% identity with SEQ ID NO: 26. In some embodiments, the variable domain of the first moiety comprises: a CDR2 amino acid sequence with at least 90% identity with SEQ ID NO: 26. In some embodiments, the variable domain of the first moiety comprises: a CDR2 amino acid sequence with at least 92% identity with SEQ ID NO: 26. In some embodiments, the variable domain of the first moiety comprises: a CDR2 amino acid sequence with at least 95% identity with SEQ ID NO: 26. In some embodiments, the variable domain of the first moiety comprises: a CDR2 amino acid sequence with at least 98% identity with SEQ ID NO: 26.

In some embodiments, the variable domain of the first moiety comprises: a CDR2 amino acid sequence with at least 75% identity with SEQ ID NO: 27. In some embodiments, the variable domain of the first moiety comprises: a CDR2 amino acid sequence with at least 80% identity with SEQ ID NO: 27. In some embodiments, the variable domain of the first moiety comprises: a CDR2 amino acid sequence with at least 85% identity with SEQ ID NO: 27. In some embodiments, the variable domain of the first moiety comprises: a CDR2 amino acid sequence with at least 88% identity with SEQ ID NO: 27. In some embodiments, the variable domain of the first moiety comprises: a CDR2 amino acid sequence with at least 90% identity with SEQ ID NO: 27. In some embodiments, the variable domain of the first moiety comprises: a CDR2 amino acid sequence with at least 92% identity with SEQ ID NO: 27. In some embodiments, the variable domain of the first moiety comprises: a CDR2 amino acid sequence with at least 95% identity with SEQ ID NO: 27. In some embodiments, the variable domain of the first moiety comprises: a CDR2 amino acid sequence with at least 98% identity with SEQ ID NO: 27.

In some embodiments, the variable domain of the first moiety comprises up to one, two, three, four or five amino acid substitutions in one or more of the CDR sequences of SEQ ID NO: 16-27.

In some embodiments, the variable domain of the first moiety comprises one amino acid substitution in CDR1 of SEQ ID NO: 16. In some embodiments, the variable domain of the first moiety comprises two amino acid substitutions in CDR1 of SEQ ID NO: 16. In some embodiments, the variable domain of the first moiety comprises three amino acid substitutions in CDR1 of SEQ ID NO: 16. In some embodiments, the variable domain of the first moiety comprises four amino acid substitutions in CDR1 of SEQ ID NO: 16. In some embodiments, the variable domain of the first moiety comprises five amino acid substitutions in CDR1 of SEQ ID NO: 16.

In some embodiments, the variable domain of the first moiety comprises one amino acid substitution in CDR2 of SEQ ID NO: 17. In some embodiments, the variable domain of the first moiety comprises two amino acid substitutions in CDR2 of SEQ ID NO: 17. In some embodiments, the variable domain of the first moiety comprises three amino acid substitutions in CDR2 of SEQ ID NO: 17. In some embodiments, the variable domain of the first moiety comprises four amino acid substitutions in CDR2 of SEQ ID NO: 17. In some embodiments, the variable domain of the first moiety comprises five amino acid substitutions in CDR2 of SEQ ID NO: 17.

In some embodiments, the variable domain of the first moiety comprises one amino acid substitution in CDR3 of SEQ ID NO: 18. In some embodiments, the variable domain of the first moiety comprises two amino acid substitutions in CDR3 of SEQ ID NO: 18. In some embodiments, the variable domain of the first moiety comprises three amino acid substitutions in CDR3 of SEQ ID NO: 18. In some embodiments, the variable domain of the first moiety comprises four amino acid substitutions in CDR3 of SEQ ID NO: 18. In some embodiments, the variable domain of the first moiety comprises five amino acid substitutions in CDR3 of SEQ ID NO: 18.

In some embodiments, the variable domain of the first moiety comprises one amino acid substitution in CDR1 of SEQ ID NO: 19. In some embodiments, the variable domain of the first moiety comprises two amino acid substitutions in CDR1 of SEQ ID NO: 19. In some embodiments, the variable domain of the first moiety comprises three amino acid substitutions in CDR1 of SEQ ID NO: 19. In some embodiments, the variable domain of the first moiety comprises four amino acid substitutions in CDR1 of SEQ ID NO: 19. In some embodiments, the variable domain of the first moiety comprises five amino acid substitutions in CDR1 of SEQ ID NO: 19.

In some embodiments, the variable domain of the first moiety comprises one amino acid substitution in CDR2 of SEQ ID NO: 20. In some embodiments, the variable domain of the first moiety comprises two amino acid substitutions in CDR2 of SEQ ID NO: 20. In some embodiments, the variable domain of the first moiety comprises three amino acid substitutions in CDR2 of SEQ ID NO: 20. In some embodiments, the variable domain of the first moiety comprises four amino acid substitutions in CDR2 of SEQ ID NO: 20. In some embodiments, the variable domain of the first moiety comprises five amino acid substitutions in CDR2 of SEQ ID NO: 20.

In some embodiments, the variable domain of the first moiety comprises one amino acid substitution in CDR2 of SEQ ID NO: 21. In some embodiments, the variable domain of the first moiety comprises two amino acid substitutions in CDR2 of SEQ ID NO: 21. In some embodiments, the variable domain of the first moiety comprises three amino acid substitutions in CDR2 of SEQ ID NO: 21. In some embodiments, the variable domain of the first moiety comprises four amino acid substitutions in CDR2 of SEQ ID NO: 21. In some embodiments, the variable domain of the first moiety comprises five amino acid substitutions in CDR2 of SEQ ID NO: 21.

In some embodiments, the variable domain of the first moiety comprises one amino acid substitution in CDR3 of SEQ ID NO: 22. In some embodiments, the variable domain of the first moiety comprises two amino acid substitutions in CDR3 of SEQ ID NO: 22. In some embodiments, the variable domain of the first moiety comprises three amino acid substitutions in CDR3 of SEQ ID NO: 22. In some embodiments, the variable domain of the first moiety comprises four amino acid substitutions in CDR3 of SEQ ID NO: 22. In some embodiments, the variable domain of the first moiety comprises five amino acid substitutions in CDR3 of SEQ ID NO: 22.

In some embodiments, the variable domain of the first moiety comprises one amino acid substitution in CDR2 of SEQ ID NO: 23. In some embodiments, the variable domain of the first moiety comprises two amino acid substitutions in CDR2 of SEQ ID NO: 23. In some embodiments, the variable domain of the first moiety comprises three amino acid substitutions in CDR2 of SEQ ID NO: 23. In some embodiments, the variable domain of the first moiety comprises four amino acid substitutions in CDR2 of SEQ ID NO: 23. In some embodiments, the variable domain of the first moiety comprises five amino acid substitutions in CDR2 of SEQ ID NO: 23.

In some embodiments, the variable domain of the first moiety comprises one amino acid substitution in CDR2 of SEQ ID NO: 24. In some embodiments, the variable domain of the first moiety comprises two amino acid substitutions in CDR2 of SEQ ID NO: 24. In some embodiments, the variable domain of the first moiety comprises three amino acid substitutions in CDR2 of SEQ ID NO: 24. In some embodiments, the variable domain of the first moiety comprises four amino acid substitutions in CDR2 of SEQ ID NO: 24. In some embodiments, the variable domain of the first moiety comprises five amino acid substitutions in CDR2 of SEQ ID NO: 24.

In some embodiments, the variable domain of the first moiety comprises one amino acid substitution in CDR2 of SEQ ID NO: 25. In some embodiments, the variable domain of the first moiety comprises two amino acid substitutions in CDR2 of SEQ ID NO: 25. In some embodiments, the variable domain of the first moiety comprises three amino acid substitutions in CDR2 of SEQ ID NO: 25. In some embodiments, the variable domain of the first moiety comprises four amino acid substitutions in CDR2 of SEQ ID NO: 25. In some embodiments, the variable domain of the first moiety comprises five amino acid substitutions in CDR2 of SEQ ID NO: 25.

In some embodiments, the variable domain of the first moiety comprises one amino acid substitution in CDR2 of SEQ ID NO: 26. In some embodiments, the variable domain of the first moiety comprises two amino acid substitutions in CDR2 of SEQ ID NO: 26. In some embodiments, the variable domain of the first moiety comprises three amino acid substitutions in CDR2 of SEQ ID NO: 26. In some embodiments, the variable domain of the first moiety comprises four amino acid substitutions in CDR2 of SEQ ID NO: 26. In some embodiments, the variable domain of the first moiety comprises five amino acid substitutions in CDR2 of SEQ ID NO: 26.

In some embodiments, the variable domain of the first moiety comprises one amino acid substitution in CDR2 of SEQ ID NO: 27. In some embodiments, the variable domain of the first moiety comprises two amino acid substitutions in CDR2 of SEQ ID NO: 27. In some embodiments, the variable domain of the first moiety comprises three amino acid substitutions in CDR2 of SEQ ID NO: 27. In some embodiments, the variable domain of the first moiety comprises four amino acid substitutions in CDR2 of SEQ ID NO: 27. In some embodiments, the variable domain of the first moiety comprises five amino acid substitutions in CDR2 of SEQ ID NO: 27.

In some embodiments, the variable domain of the first moiety comprises a CDR1 defined by SEQ ID NO: 16. In some embodiments, the variable domain of the first moiety comprises a CDR2 defined by SEQ ID NO: 17. In some embodiments, the variable domain of the first moiety comprises a CDR3 defined by SEQ ID NO: 18. In some embodiments, the variable domain of the first moiety comprises a CDR1 defined by SEQ ID NO: 19. In some embodiments, the variable domain of the first moiety comprises a CDR2 defined by SEQ ID NO: 20. In some embodiments, the variable domain of the first moiety comprises a CDR2 defined by SEQ ID NO: 21. In some embodiments, the variable domain of the first moiety comprises a CDR3 defined by SEQ ID NO: 22. In some embodiments, the variable domain of the first moiety comprises a CDR2 defined by SEQ ID NO: 23. In some embodiments, the variable domain of the first moiety comprises a CDR2 defined by SEQ ID NO: 24. In some embodiments, the variable domain of the first moiety comprises a CDR2 defined by SEQ ID NO: 25. In some embodiments, the variable domain of the first moiety comprises a CDR2 defined by SEQ ID NO: 26. In some embodiments, the variable domain of the first moiety comprises a CDR2 defined by SEQ ID NO: 27.

In some embodiments, the variable domain of the first moiety comprises a CDR1 defined by SEQ ID NO: 16, a CDR2 defined by SEQ ID NO: 17, and a CDR3 defined by SEQ ID NO: 18. In some embodiments, the variable domain of the first moiety comprises a CDR1 defined by SEQ ID NO: 19, a CDR2 defined by SEQ ID NO: 20, and a CDR3 defined by SEQ ID NO: 18. In some embodiments, the variable domain of the first moiety comprises a CDR1 defined by SEQ ID NO: 16, a CDR2 defined by SEQ ID NO: 21, and a CDR3 defined by SEQ ID NO: 22. In some embodiments, the variable domain of the first moiety comprises a CDR1 defined by SEQ ID NO: 19, a CDR2 defined by SEQ ID NO: 23, and a CDR3 defined by SEQ ID NO: 22. In some embodiments, the variable domain of the first moiety comprises a CDR1 defined by SEQ ID NO: 16, a CDR2 defined by SEQ ID NO: 24, and a CDR3 defined by SEQ ID NO: 22. In some embodiments, the variable domain of the first moiety comprises a CDR1 defined by SEQ ID NO: 19, a CDR2 defined by SEQ ID NO: 25, and a CDR3 defined by SEQ ID NO: 22. In some embodiments, the variable domain of the first moiety comprises a CDR1 defined by SEQ ID NO: 16, a CDR2 defined by SEQ ID NO: 26, and a CDR3 defined by SEQ ID NO: 22. In some embodiments, the variable domain of the first moiety comprises a CDR1 defined by SEQ ID NO: 19, a CDR2 defined by SEQ ID NO: 27, and a CDR3 defined by SEQ ID NO: 22.

In some embodiments, the variable domain of the first moiety comprises the sequence of

(SEQ ID NO: 10)
EVQLLESGGGLVQPGGSLRLSCAASGRTFSNYHMGWFRQAPGQG
REFVATIIRTGETIYYADSVKGRFTISRDNSKNTLYLQMNSLRA
EDTAVYYCAAATSGWNIKTVTPYEYWGQGTLVTVSS.

In some embodiments, the variable domain of the first moiety comprises the sequence of

(SEQ ID NO: 11)
EVQLLESGGGLVQPGGSLRLSCAASGRTFSNYHMGWFRQAPGQG
REFVATIIRTGKTIYYADSVKGRFTISRDNSKNTLYLQMNSLRA
EDTAVYYCAAATSGWHIKTVTPYEYWGQGTLVTVSS.

In some embodiments, the variable domain of the first moiety comprises the sequence of

(SEQ ID NO: 12)
EVQLLESGGGLVQPGGSLRLSCAASGRTFSNYHMGWFRQAPGQG
REFVATIIRTGTTIYYADSVKGRFTISRDNSKNTLYLQMNSLRA
EDTAVYYCAAATSGWHIKTVTPYEYWGQGTLVTVSS.

In some embodiments, the variable domain of the first moiety comprises the sequence of

(SEQ ID NO: 13)
EVQLLESGGGLVQPGGSLRLSCAASGRTFSNYHMGWFRQAPGQG
REFVATIIRHGTTIYYADSVKGRFTISRDNSKNTLYLQMNSLRA
EDTAVYYCAAATSGWHIKTVTPYEYWGQGTLVTVSS.

In some embodiments, the variable domain of the first moiety comprises amino acid sequences with at least 75%, 78%, 80%, 82%, 85%, 87%, 90%, 91%, 92%, 93%, 94%, 95%, 96%, 97%, 98%, 99% or more identity with one or more of SEQ ID NO: 10-13.

In some embodiments, the variable domain of the first moiety comprises amino acid sequence with at least 75% identity to SEQ ID NO: 10. In some embodiments, the variable domain of the first moiety comprises amino acid sequence with at least 80% identity to SEQ ID NO: 10. In some embodiments, the variable domain of the first moiety comprises amino acid sequence with at least 82% identity to SEQ ID NO: 10. In some embodiments, the variable domain of the first moiety comprises amino acid sequence with at least 85% identity to SEQ ID NO: 10. In some embodiments, the variable domain of the first moiety comprises amino acid sequence with at least 88% identity to SEQ ID NO: 10. In some embodiments, the variable domain of the first moiety comprises amino acid sequence with at least 90% identity to SEQ ID NO: 10. In some embodiments, the variable domain of the first moiety comprises amino acid sequence with at least 91% identity to SEQ ID NO: 10. In some embodiments, the variable domain of the first moiety comprises amino acid sequence with at least 92% identity to SEQ ID NO: 10. In some embodiments, the variable domain of the first moiety comprises amino acid sequence with at least 93% identity to SEQ ID NO: 10. In some embodiments, the variable domain of the first moiety comprises amino acid sequence with at least 94% identity to SEQ ID NO: 10. In some embodiments, the variable domain of the first moiety comprises amino acid sequence with at least 95% identity to SEQ ID NO: 10. In some embodiments, the variable domain of the first moiety comprises amino acid sequence with at least 96% identity to SEQ ID NO: 10. In some embodiments, the variable domain of the first moiety comprises amino acid sequence with at least 97% identity to SEQ ID NO: 10. In some embodiments, the variable domain of the first moiety comprises amino acid sequence with at least 98% identity to SEQ ID NO: 10. In some embodiments, the variable domain of the first moiety comprises amino acid sequence with at least 99% identity to SEQ ID NO: 10. In some embodiments, the variable domain of the first moiety comprises amino acid sequence of SEQ ID NO: 10.

In some embodiments, the variable domain of the first moiety comprises amino acid sequence with at least 75% identity to SEQ ID NO: 11. In some embodiments, the variable domain of the first moiety comprises amino acid sequence with at least 80% identity to SEQ ID NO: 11. In some embodiments, the variable domain of the first moiety comprises amino acid sequence with at least 82% identity to SEQ ID NO: 11. In some embodiments, the variable domain of the first moiety comprises amino acid sequence with at least 85% identity to SEQ ID NO: 11. In some embodiments, the variable domain of the first moiety comprises amino acid sequence with at least 88% identity to SEQ ID NO: 11. In some embodiments, the variable domain of the first moiety comprises amino acid sequence with at least 90% identity to SEQ ID NO: 11. In some embodiments, the variable domain of the first moiety comprises amino acid sequence with at least 91% identity to SEQ ID NO: 11. In some embodiments, the variable domain of the first moiety comprises amino acid sequence with at least 92% identity to SEQ ID NO: 11. In some embodiments, the variable domain of the first moiety comprises amino acid sequence with at least 93% identity to SEQ ID NO: 11. In some embodiments, the variable domain of the first moiety comprises amino acid sequence with at least 94% identity to SEQ ID NO: 11. In some embodiments, the variable domain of the first moiety comprises amino acid sequence with at least 95% identity to SEQ ID NO: 11. In some embodiments, the variable domain of the first moiety comprises amino acid sequence with at least 96% identity to SEQ ID NO: 11. In some embodiments, the variable domain of the first moiety comprises amino acid sequence with at least 97% identity to SEQ ID NO: 1. In some embodiments, the variable domain of the first moiety comprises amino acid sequence with at least 98% identity to SEQ ID NO: 11. In some embodiments, the variable domain of the first moiety comprises amino acid sequence with at least 99% identity to SEQ ID NO: 11. In some embodiments, the variable domain of the first moiety comprises amino acid sequence of SEQ ID NO: 11.

In some embodiments, the variable domain of the first moiety comprises amino acid sequence with at least 75% identity to SEQ ID NO: 12. In some embodiments, the variable domain of the first moiety comprises amino acid sequence with at least 80% identity to SEQ ID NO: 12. In some embodiments, the variable domain of the first moiety comprises amino acid sequence with at least 82% identity to SEQ ID NO: 12. In some embodiments, the variable domain of the first moiety comprises amino acid sequence with at least 85% identity to SEQ ID NO: 12. In some embodiments, the variable domain of the first moiety comprises amino acid sequence with at least 88% identity to SEQ ID NO: 12. In some embodiments, the variable domain of the first moiety comprises amino acid sequence with at least 90% identity to SEQ ID NO: 12. In some embodiments, the variable domain of the first moiety comprises amino acid sequence with at least 91% identity to SEQ ID NO: 12. In some embodiments, the variable domain of the first moiety comprises amino acid sequence with at least 92% identity to SEQ ID NO: 12. In some embodiments, the variable domain of the first moiety comprises amino acid sequence with at least 93% identity to SEQ ID NO: 12. In some embodiments, the variable domain of the first moiety comprises amino acid sequence with at least 94% identity to SEQ ID NO: 12. In some embodiments, the variable domain of the first moiety comprises amino acid sequence with at least 95% identity to SEQ ID NO: 12. In some embodiments, the variable domain of the first moiety comprises amino acid sequence with at least 96% identity to SEQ ID NO: 12. In some embodiments, the variable domain of the first moiety comprises amino acid sequence with at least 97% identity to SEQ ID NO: 12. In some embodiments, the variable domain of the first moiety comprises amino acid sequence with at least 98% identity to SEQ ID NO: 12. In some embodiments, the variable domain of the first moiety comprises amino acid sequence with at least 99% identity to SEQ ID NO: 12. In some embodiments, the variable domain of the first moiety comprises amino acid sequence of SEQ ID NO: 12.

In some embodiments, the variable domain of the first moiety comprises amino acid sequence with at least 75% identity to SEQ ID NO: 13. In some embodiments, the variable domain of the first moiety comprises amino acid sequence with at least 80% identity to SEQ ID NO: 13. In some embodiments, the variable domain of the first moiety comprises amino acid sequence with at least 82% identity to SEQ ID NO: 13. In some embodiments, the variable domain of the first moiety comprises amino acid sequence with at least 85% identity to SEQ ID NO: 13. In some embodiments, the variable domain of the first moiety comprises amino acid sequence with at least 88% identity to SEQ ID NO: 13. In some embodiments, the variable domain of the first moiety comprises amino acid sequence with at least 90% identity to SEQ ID NO: 13. In some embodiments, the variable domain of the first moiety comprises amino acid sequence with at least 91% identity to SEQ ID NO: 13. In some embodiments, the variable domain of the first moiety comprises amino acid sequence with at least 92% identity to SEQ ID NO: 13. In some embodiments, the variable domain of the first moiety comprises amino acid sequence with at least 93% identity to SEQ ID NO: 13. In some embodiments, the variable domain of the first moiety comprises amino acid sequence with at least 94% identity to SEQ ID NO: 13. In some embodiments, the variable domain of the first moiety comprises amino acid sequence with at least 95% identity to SEQ ID NO: 13. In some embodiments, the variable domain of the first moiety comprises amino acid sequence with at least 96% identity to SEQ ID NO: 13. In some embodiments, the variable domain of the first moiety comprises amino acid sequence with at least 97% identity to SEQ ID NO: 13. In some embodiments, the variable domain of the first moiety comprises amino acid sequence with at least 98% identity to SEQ ID NO: 13. In some embodiments, the variable domain of the first moiety comprises amino acid sequence with at least 99% identity to SEQ ID NO: 13. In some embodiments, the variable domain of the first moiety comprises amino acid sequence of SEQ ID NO: 13.

### Membrane Cofactor Protein (MCP)

MCP (also known as CD46) has of four main domains: four short consensus repeats (SCRs) of complement control protein (CCP) modules, also known as sushi domains at the N-terminus, a serine/threonine/proline (STP)-rich region, a transmembrane domain, and a cytoplasmic tail; with the majority of its function occurring within the extracellular SCR domains responsible for binding to complement proteins like C3b and C4b to protect host cells from complement-mediated damage.

In some embodiments, a polypeptide that is capable of binding to C3b comprises a second moiety comprising an amino acid sequence of MCP or a variant thereof. In some embodiments, a second moiety comprises domains 2-4 of MCP. In some embodiments, a second moiety comprises amino acids residues 96-285 of SEQ ID NO: 14.

MCP comprises an amino acid sequence of:

(SEQ ID NO: 14)
CEEPPTFEAMELIGKPKPYYEIGERVDYKCKKGYFYIPPLATHT
ICDRNHTWLPVSDDACYRETCPYIRDPLNGQAVPANGTYEFGYQ
MHFICNEGYYLIGEEILYCELKGSVAIWSGKPPICEKVLCTPPP
KIKNGKHTFSEVEVFEYLDAVTYSCDPAPGPDPFSLIGESTIYC
GDNSVWSRAAPECKVVKCRFPVVENGKQISGFGKKFYYKATVMF
ECDKGFYLDGSDTIVCDSNSTWDPPVPKCLKVLPPSSTKPPALS
HSVSTSSTTKSPASSASGPRPTYKPPVSNYPGYPKPEEGILDSL
DVWVIAVIVIAIVVGVAVICVVPYRYLQRRKKKGTYLTDETHRE
VKFTSL.

Domains 2-4 of MCP comprises an amino acid sequence of:

(SEQ ID NO: 9)
RETCPYIRDPLNGQAVPANGTYEFGYQMHFICNEGYYLIGEEIL
YCELKGSVAIWSGKPPICEKVLCTPPPKIKNGKHTFSEVEVFEY
LDAVTYSCDPAPGPDPFSLIGESTIYCGDNSVWSRAAPECKVVK
CRFPVVENGKQISGFGKKFYYKATVMFECDKGFYLDGSDTIVCD
SNSTWDPPVPKCLKV.

In some embodiments, a second moiety comprises an amino acid sequence of MCP having at least 70% identity to SEQ ID NO: 14. In some embodiments, a second moiety comprises an amino acid sequence of MCP having at least 75% identity to SEQ ID NO: 14. In some embodiments, a second moiety comprises an amino acid sequence of MCP having at least 78% identity to SEQ ID NO: 14. In some embodiments, a second moiety comprises an amino acid sequence of MCP having at least 80% identity to SEQ ID NO: 14. In some embodiments, a second moiety comprises an amino acid sequence of MCP having at least 82% identity to SEQ ID NO: 14. In some embodiments, a second moiety comprises an amino acid sequence of MCP having at least 85% identity to SEQ ID NO: 14. In some embodiments, a second moiety comprises an amino acid sequence of MCP having at least 88% identity to SEQ ID NO: 14. In some embodiments, a second moiety comprises an amino acid sequence of MCP having at least 90% identity to SEQ ID NO: 14. In some embodiments, a second moiety comprises an amino acid sequence of MCP having at least 91% identity to SEQ ID NO: 14. In some embodiments, a second moiety comprises an amino acid sequence of MCP having at least 92% identity to SEQ ID NO: 14. In some embodiments, a second moiety comprises an amino acid sequence of MCP having at least 93% identity to SEQ ID NO: 14. In some embodiments, a second moiety comprises an amino acid sequence of MCP having at least 94% identity to SEQ ID NO: 14. In some embodiments, a second moiety comprises an amino acid sequence of MCP having at least 95% identity to SEQ ID NO: 14. In some embodiments, a second moiety comprises an amino acid sequence of MCP having at least 96% identity to SEQ ID NO: 14. In some embodiments, a second moiety comprises an amino acid sequence of MCP having at least 97% identity to SEQ ID NO: 14. In some embodiments, a second moiety comprises an amino acid sequence of MCP having at least 98% identity to SEQ ID NO: 14. In some embodiments, a second moiety comprises an amino acid sequence of MCP having at least 99% identity to SEQ ID NO: 14. In some embodiments, a second moiety comprises an amino acid sequence of MCP comprising SEQ ID NO: 14.

In some embodiments, a second moiety comprises an amino acid sequence of MCP having at least 70% identity to SEQ ID NO: 9. In some embodiments, a second moiety comprises an amino acid sequence of MCP having at least 75% identity to SEQ ID NO: 9. In some embodiments, a second moiety comprises an amino acid sequence of MCP having at least 78% identity to SEQ ID NO: 9. In some embodiments, a second moiety comprises an amino acid sequence of MCP having at least 80% identity to SEQ ID NO: 9. In some embodiments, a second moiety comprises an amino acid sequence of MCP having at least 82% identity to SEQ ID NO: 9. In some embodiments, a second moiety comprises an amino acid sequence of MCP having at least 85% identity to SEQ ID NO: 9. In some embodiments, a second moiety comprises an amino acid sequence of MCP having at least 88% identity to SEQ ID NO: 9. In some embodiments, a second moiety comprises an amino acid sequence of MCP having at least 90% identity to SEQ ID NO: 9. In some embodiments, a second moiety comprises an amino acid sequence of MCP having at least 91% identity to SEQ ID NO: 9. In some embodiments, a second moiety comprises an amino acid sequence of MCP having at least 92% identity to SEQ ID NO: 9. In some embodiments, a second moiety comprises an amino acid sequence of MCP having at least 93% identity to SEQ ID NO: 9. In some embodiments, a second moiety comprises an amino acid sequence of MCP having at least 94% identity to SEQ ID NO: 9. In some embodiments, a second moiety comprises an amino acid sequence of MCP having at least 95% identity to SEQ ID NO: 9. In some embodiments, a second moiety comprises an amino acid sequence of MCP having at least 96% identity to SEQ ID NO: 9. In some embodiments, a second moiety comprises an amino acid sequence of MCP having at least 97% identity to SEQ ID NO: 9. In some embodiments, a second moiety comprises an amino acid sequence of MCP having at least 98% identity to SEQ ID NO: 9. In some embodiments, a second moiety comprises an amino acid sequence of MCP having at least 99% identity to SEQ ID NO: 9. In some embodiments, a second moiety comprises an amino acid sequence of MCP comprising SEQ ID NO: 9.

In some embodiments, the MCP comprises one or more amino acid modifications.

In some embodiments, the MCP variant comprises the amino acid modification of S177R (corresponds to S238R in MCP full length sequence). relative to SEQ ID NO: 9.

In some embodiments, a second moiety comprises an amino acid sequence of MCP comprising S177R substitution relative to SEQ ID NO: 9:

(SEQ ID NO: 15)
RETCPYIRDPLNGQAVPANGTYEFGYQMHFICNEGYYLIGEEILYCELK
GSVAIWSGKPPICEKVLCTPPPKIKNGKHTFSEVEVFEYLDAVTYSCDP
APGPDPFSLIGESTIYCGDNSVWSRAAPECKVVKCRFPVVENGKQISGF
GKKFYYKATVMFECDKGFYLDGSDTIVCDRNSTWDPPVPKCLKV.

In some embodiments, a second moiety comprises an amino acid sequence of MCP having at least 70% identity to SEQ ID NO: 15. In some embodiments, a second moiety comprises an amino acid sequence of MCP having at least 75% identity to SEQ ID NO: 15. In some embodiments, a second moiety comprises an amino acid sequence of MCP having at least 78% identity to SEQ ID NO: 15. In some embodiments, a second moiety comprises an amino acid sequence of MCP having at least 80% identity to SEQ ID NO: 15. In some embodiments, a second moiety comprises an amino acid sequence of MCP having at least 82% identity to SEQ ID NO: 15. In some embodiments, a second moiety comprises an amino acid sequence of MCP having at least 85% identity to SEQ ID NO: 15. In some embodiments, a second moiety comprises an amino acid sequence of MCP having at least 88% identity to SEQ ID NO: 15. In some embodiments, a second moiety comprises an amino acid sequence of MCP having at least 90% identity to SEQ ID NO: 15. In some embodiments, a second moiety comprises an amino acid sequence of MCP having at least 91% identity to SEQ ID NO: 15. In some embodiments, a second moiety comprises an amino acid sequence of MCP having at least 92% identity to SEQ ID NO: 15. In some embodiments, a second moiety comprises an amino acid sequence of MCP having at least 93% identity to SEQ ID NO: 15. In some embodiments, a second moiety comprises an amino acid sequence of MCP having at least 94% identity to SEQ ID NO: 15. In some embodiments, a second moiety comprises an amino acid sequence of MCP having at least 95% identity to SEQ ID NO: 15. In some embodiments, a second moiety comprises an amino acid sequence of MCP having at least 96% identity to SEQ ID NO: 15. In some embodiments, a second moiety comprises an amino acid sequence of MCP having at least 97% identity to SEQ ID NO: 15. In some embodiments, a second moiety comprises an amino acid sequence of MCP having at least 98% identity to SEQ ID NO: 15. In some embodiments, a second moiety comprises an amino acid sequence of MCP having at least 99% identity to SEQ ID NO: 9. In some embodiments, a second moiety comprises an amino acid sequence of MCP comprising SEQ ID NO: 15.

In some embodiments, C-terminus of the first moiety is linked to the N-terminus of the second moiety. In some embodiments, N-terminus of the first moiety is linked to the C-terminus of the second moiety.

### Half-Life Extension Domain

In some embodiments, the polypeptide further comprises a third moiety comprising a half-life extension domain. In some embodiments, the half-life extension domain comprises an Fc domain, an albumin binding domain, a human serum albumin, one or more lipids, one or more polyethylene glycols (PEGs), or any other half-life extending polypeptide or polymer known in the art.

In some embodiments, the half-life extension domain comprises a serum protein binding moiety. In some embodiments, the half-life extension domain comprises human serum albumin. In some embodiments, the half-life extension domain comprises one or more lipids. In some embodiments, the half-life extension domain comprises one or more half-life extending polymers. In some embodiments, the half-life extension domain comprises one or more PEGs. In some embodiments, the half-life extension domain comprises a half-life extending polypeptide.

In some embodiments, the half-life extension moiety is a Fc domain. The Fc domain is the constant region (Fc) of immunoglobulin. The Fc domain can be derived from IgG1, IgG2, IgG3 or IgG4. In one embodiment, the Fc domain is from IgG4 or a variant thereof.

### Fc Domain

As used herein, the “Fc domain” and “Fc region” are used interchangeably. The Fc domain of an antibody interacts with a number of Fc receptors and ligands, imparting an array of important functional capabilities referred to as effector functions. For IgG, the Fc region comprises Ig domains Cγ2 and Cγ3 and the N-terminal hinge leading into Cγ2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, incorporated by reference). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and γδ T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290, incorporated by reference). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). A number of structures have been solved of the extracellular domains of human FcγRs, including FcγRIIa (pdb accession code 1H9V) (Sondermann et al., 2001, J Mol Biol 309:737-749) (pdb accession code 1 FCG) (Maxwell et al., 1999, Nat Struct Biol 6:437-442), FcγRIIb (pdb accession code 2FCB) (Sondermann et al., 1999, Embo J 18:1095-1103); and FcγRIIIb (pdb accession code 1E4J) (Sondermann et al., 2000, Nature 406:267-273, incorporated by reference). All FcγRs bind the same region on Fc, at the N-terminal end of the Cγ2 domain and the preceding hinge. This interaction is well characterized structurally (Sondermann et al., 2001, J Mol Biol 309:737-749 incorporated by reference), and several structures of the human Fc bound to the extracellular domain of human FcγRIIIb have been solved (pdb accession code 1E4K)(Sondermann et al., 2000, Nature 406:267-273) (pdb accession codes 1IIS and 1IIX) (Radaev et al., 2001, J Biol Chem 276:16469-16477, incorporated by reference), as well as has the structure of the human IgE Fd/FcεRIa complex (pdb accession code 1F6A) (Garman et al., 2000, Nature 406:259-266, incorporated by reference).

An overlapping but separate site on Fc serves as the interface for the complement protein C1q. In the same way that Fc/FcγR binding mediates ADCC, Fc/C1q binding mediates complement dependent cytotoxicity (CDC). C1q forms a complex with the serine proteases C1r and C1s to form the C1 complex. C1q is capable of binding six antibodies, although binding to two IgGs is sufficient to activate the complement cascade. Similar to Fc interaction with FcγRs, different IgG subclasses have different affinity for C1q, with IgG1 and IgG3 typically binding substantially better to the FcγRs than IgG2 and IgG4.

A site on Fc between the Cγ2 and Cγ3 domains mediates interaction with the neonatal receptor FcRn, the binding of which recycles endocytosed antibody from the endosome back to the bloodstream (Raghavan et al., 1996, Annu Rev Cell Dev Biol 12:181-220; Ghetie et al., 2000, Annu Rev Immunol 18:739-766, incorporated by reference). This process, coupled with preclusion of kidney filtration due to the large size of the full-length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. Thus the fidelity of this region on Fc is important for both the clinical properties of antibodies and their purification. Available structures of the rat Fc/FcRn complex (Martin et al., 2001, Mol Cell 7:867-877, incorporated by reference), and of the complexes of Fc with proteins A and G (Deisenhofer, 1981, Biochemistry 20:2361-2370; Sauer-Eriksson et al., 1995, Structure 3:265-278; Tashiro et al., 1995, Curr Opin Struct Biol 5:471-481, incorporated by reference) provide insight into the interaction of Fc with these proteins.

In some embodiments, the third moiety is a Fe variant. Fe variants described herein are optimized for a number of therapeutically relevant properties. An Fe variant comprises one or more amino acid modifications relative to a parent Fc region, wherein said amino acid modification(s) provide one or more optimized properties. In some embodiments, Fe variants have at least one amino acid modification compared to the parent. Alternatively, in some embodiments the Fe variants may have more than one amino acid modification as compared to the parent, for example from about one to fifty amino acid modifications, from about one to ten amino acid modifications, or from about one to about five amino acid modifications compared to the parent. Thus, the sequences of the Fe variants and those of the parent Fc polypeptide are substantially homologous. For example, the variant Fe variant sequences herein will possess about 80% homology with the parent Fe variant sequence, preferably at least about 90% homology, and most preferably at least about 95% homology.

The Fe variants may be optimized for a variety of properties. An Fe variant that is engineered or predicted to display one or more optimized properties is herein referred to as an “optimized Fe variant”. Properties that may be optimized include but are not limited to enhanced or reduced affinity for an FcγR. In some embodiments, the Fe variants are optimized to have reduced or ablated affinity for a human FcγR, including but not limited to FcγRI, FcγRIIa, FcγRIIb, FcγRIIc, FcγRIIIa, and FcγRIIIb. These embodiments are anticipated to provide Fc polypeptides with enhanced therapeutic properties in humans, for example reduced effector function and reduced toxicity. In other embodiments, Fe variants provide enhanced affinity for one or more FcγRs, yet reduced affinity for one or more other FcγRs. For example, an Fe variant may have enhanced binding to FcγRIIIa, yet reduced binding to FcγRIIb. Alternately, an Fe variant may have enhanced binding to FcγRIIa and FcγRI, yet reduced binding to FcγRIIb. In yet another embodiment, an Fe variant may have enhanced affinity for FcγRIIb, yet reduced affinity to one or more activating FcγRs.

In some embodiments, an Fe variant has reduced or ablated affinity for FcγRI. In some embodiments, an Fe variant has reduced or ablated affinity for FcγRIIa. In some embodiments, an Fe variant has reduced or ablated affinity for FcγRIIb. In some embodiments, an Fe variant has reduced or ablated affinity for FcγRIIc. In some embodiments, an Fe variant has reduced or ablated affinity for FcγRIIIa. In some embodiments, an Fe variant has reduced or ablated affinity for FcγRIIIb. In some embodiments, an Fe variant has reduced or ablated affinity for C1q. In some embodiments, an Fe variant has enhanced affinity for FcRn. In some embodiments, an Fe variant maintains affinity for FcRn. In some embodiments, an Fe variant has reduced or ablated affinity for FcγRI, FcγRIIa, FcγRIIb, FcγRIIIa, FcγRIIIb, and C1q. In some embodiments, an Fe variant has reduced or ablated affinity for FcγRI, FcγRIIa, FcγRIIb, FcγRIIIa, FcγRIIIb, and C1q, and retains binding to FcRn.

The Fe variants may also be optimized for enhanced functionality and/or solution properties in aglycosylated form. In a preferred embodiment, the aglycosylated Fe variants bind an Fc ligand with reduced affinity than the aglycosylated form of the parent Fe variant. Said Fc ligands include but are not limited to FcγRs, C1q, FcRn, and proteins A and G, and may be from any source including but not limited to human, mouse, rat, rabbit, or monkey, preferably human. In an alternately preferred embodiment, the Fe variants are optimized to be more stable and/or more soluble than the aglycosylated form of the parent Fe variant.

In some embodiments, the Fc domain is derived from IgG1. In some embodiments, the Fc domain is derived from IgG1 allotype nG1m(a) (also known as nG1m1 or G1m(f)). In some embodiments, the Fc domain is derived from IgG2. In some embodiments, the Fc domain is derived from IgG4.

In some embodiments, the Fc domain is an Fe variant comprising one or more amino acid modifications.

In some embodiments, the Fc domain is derived from IgG4 and comprises an amino acid modification of S228P.

In some embodiments, the Fc domain comprises the amino acid modification of T307Q. In some embodiments, the Fc domain comprises an amino acid modification of Q311V. In some embodiments, the Fc domain comprises an amino acid modification of A378V. In some embodiments, the Fc domain comprises the amino acid modifications of T307Q, Q311V and A378V. In some embodiments, the Fc domain is derived from IgG4 and comprises amino acid modifications S228P, T307Q, Q311V and A378V.

In some embodiments, the Fc domain comprises an amino acid modification of 234V. In some embodiments, the Fc domain comprises an amino acid modification of L235E. In some embodiments, the Fc domain comprises an amino acid modification of D265G. In some embodiments, the Fc domain comprises amino acid modifications of 234V, L235E, and D265G. In some embodiments, the Fc domain comprises at least one amino acid modification from 234V, L235E and D265G. In some embodiments, the Fc domain is derived from IgG4 and comprises amino acid modifications S228P, F234V, L235E and D265G.

In some embodiments, the Fc domain comprises amino acid modifications 234V, L235E and D265G, T307Q, Q311V and A378V. In some embodiments, the Fc domain comprises amino acid modifications S228P, F234V, L235E and D265G, T307Q, Q311V and A378V.

In some embodiments, the Fc domain comprises the amino acid sequence of SEQ ID: 28.

(SEQ ID NO: 28)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG

In some embodiments, the polypeptide comprises an Fc domain having an amino acid sequence with at least 80% identity to SEQ ID NO: 28. In some embodiments, the polypeptide comprises an Fc domain having an amino acid sequence with at least 82% identity to SEQ ID NO: 28. In some embodiments, the polypeptide comprises an Fc domain having an amino acid sequence with at least 85% identity to SEQ ID NO: 28. In some embodiments, the polypeptide comprises an Fc domain having an amino acid sequence with at least 88% identity to SEQ ID NO: 28. In some embodiments, the polypeptide comprises an Fc domain having an amino acid sequence with at least 90% identity to SEQ ID NO: 28. In some embodiments, the polypeptide comprises an Fc domain having an amino acid sequence with at least 92% identity to SEQ ID NO: 28. In some embodiments, the polypeptide comprises an Fc domain having an amino acid sequence with at least 95% identity to SEQ ID NO: 28. In some embodiments, the polypeptide comprises an Fc domain having an amino acid sequence with at least 96% identity to SEQ ID NO: 28. In some embodiments, the polypeptide comprises an Fc domain having an amino acid sequence with at least 97% identity to SEQ ID NO: 28. In some embodiments, the polypeptide comprises an Fc domain having an amino acid sequence with at least 98% identity to SEQ ID NO: 28. In some embodiments, the polypeptide comprises an Fc domain having an amino acid sequence with at least 99% identity to SEQ ID NO: 28.

In some embodiments, the Fc domain comprises the amino acid sequence of SEQ ID: 29.

(SEQ ID NO: 29)
DKTVERKCCVECPPCPAPPVAGPSVFLFPPKPKDTLMISRTPEVTCVVV
DVSHEDPEVQFNWYVDGVEVHNAKTKPREEQFNSTFRVVSVLTVVHQDW
LNGKEYKCKVSNKGLPAPIEKTISKTKGQPREPQVYTLPPSREEMTKNQ
VSLTCLVKGFYPSDISVEWESNGQPENNYKTTPPMLDSDGSFFLYSKLT
VDKSRWQQGNVFSCSVMHEALHNHYTQKSLSLSPG

In some embodiments, the polypeptide comprises an Fc domain having an amino acid sequence with at least 80% identity to SEQ ID NO: 29. In some embodiments, the polypeptide comprises an Fc domain having an amino acid sequence with at least 82% identity to SEQ ID NO: 29. In some embodiments, the polypeptide comprises an Fc domain having an amino acid sequence with at least 85% identity to SEQ ID NO: 29. In some embodiments, the polypeptide comprises an Fc domain having an amino acid sequence with at least 88% identity to SEQ ID NO: 29. In some embodiments, the polypeptide comprises an Fc domain having an amino acid sequence with at least 90% identity to SEQ ID NO: 29. In some embodiments, the polypeptide comprises an Fc domain having an amino acid sequence with at least 92% identity to SEQ ID NO: 29. In some embodiments, the polypeptide comprises an Fc domain having an amino acid sequence with at least 95% identity to SEQ ID NO: 29. In some embodiments, the polypeptide comprises an Fc domain having an amino acid sequence with at least 96% identity to SEQ ID NO: 29. In some embodiments, the polypeptide comprises an Fc domain having an amino acid sequence with at least 97% identity to SEQ ID NO: 29. In some embodiments, the polypeptide comprises an Fc domain having an amino acid sequence with at least 98% identity to SEQ ID NO: 29. In some embodiments, the polypeptide comprises an Fc domain having an amino acid sequence with at least 99% identity to SEQ ID NO: 29.

In some embodiments, the Fc domain comprises the amino acid sequence of SEQ ID: 30.

(SEQ ID NO: 30)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLG

In some embodiments, the polypeptide comprises an Fc domain having an amino acid sequence with at least 80% identity to SEQ ID NO: 30. In some embodiments, the polypeptide comprises an Fc domain having an amino acid sequence with at least 82% identity to SEQ ID NO: 30. In some embodiments, the polypeptide comprises an Fc domain having an amino acid sequence with at least 85% identity to SEQ ID NO: 30. In some embodiments, the polypeptide comprises an Fc domain having an amino acid sequence with at least 88% identity to SEQ ID NO: 30. In some embodiments, the polypeptide comprises an Fc domain having an amino acid sequence with at least 90% identity to SEQ ID NO: 30. In some embodiments, the polypeptide comprises an Fc domain having an amino acid sequence with at least 92% identity to SEQ ID NO: 30. In some embodiments, the polypeptide comprises an Fc domain having an amino acid sequence with at least 95% identity to SEQ ID NO: 30. In some embodiments, the polypeptide comprises an Fc domain having an amino acid sequence with at least 96% identity to SEQ ID NO: 30. In some embodiments, the polypeptide comprises an Fc domain having an amino acid sequence with at least 97% identity to SEQ ID NO: 30. In some embodiments, the polypeptide comprises an Fc domain having an amino acid sequence with at least 98% identity to SEQ ID NO: 30. In some embodiments, the polypeptide comprises an Fc domain having an amino acid sequence with at least 99% identity to SEQ ID NO: 30.

In some embodiments, the Fc domain comprises the amino acid sequence of SEQ ID: 31.

(SEQ ID NO: 31)
ESKYGPPCPPCPAPEVEGGPSVFLFPPKPKDTLMISRTPEVTCVVVGVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLG

In some embodiments, the polypeptide comprises an Fc domain having an amino acid sequence with at least 80% identity to SEQ ID NO: 31. In some embodiments, the polypeptide comprises an Fc domain having an amino acid sequence with at least 82% identity to SEQ ID NO: 31. In some embodiments, the polypeptide comprises an Fc domain having an amino acid sequence with at least 85% identity to SEQ ID NO: 31. In some embodiments, the polypeptide comprises an Fc domain having an amino acid sequence with at least 88% identity to SEQ ID NO: 31. In some embodiments, the polypeptide comprises an Fc domain having an amino acid sequence with at least 90% identity to SEQ ID NO: 31. In some embodiments, the polypeptide comprises an Fc domain having an amino acid sequence with at least 92% identity to SEQ ID NO: 31. In some embodiments, the polypeptide comprises an Fc domain having an amino acid sequence with at least 95% identity to SEQ ID NO: 31. In some embodiments, the polypeptide comprises an Fc domain having an amino acid sequence with at least 96% identity to SEQ ID NO: 31. In some embodiments, the polypeptide comprises an Fc domain having an amino acid sequence with at least 97% identity to SEQ ID NO: 31. In some embodiments, the polypeptide comprises an Fc domain having an amino acid sequence with at least 98% identity to SEQ ID NO: 31. In some embodiments, the polypeptide comprises an Fc domain having an amino acid sequence with at least 99% identity to SEQ ID NO: 31.

In some embodiments, the Fc domain comprises the amino acid sequence of SEQ ID: 32.

(SEQ ID NO: 32)
ESKYGPPCPPCPAPEFLGGPSVFLFPPKPKDTLMISRTPEVTCVVVDVS
QEDPEVQFNWYVDGVEVHNAKTKPREEQFNSTYRVVSVLQVLHVDWLNG
KEYKCKVSNKGLPSSIEKTISKAKGQPREPQVYTLPPSQEEMTKNQVSL
TCLVKGFYPSDIVVEWESNGQPENNYKTTPPVLDSDGSFFLYSRLTVDK
SRWQEGNVFSCSVMHEALHNHYTQKSLSLSLG

In some embodiments, the polypeptide comprises an Fc domain having an amino acid sequence with at least 80% identity to SEQ ID NO: 32. In some embodiments, the polypeptide comprises an Fc domain having an amino acid sequence with at least 82% identity to SEQ ID NO: 32. In some embodiments, the polypeptide comprises an Fc domain having an amino acid sequence with at least 85% identity to SEQ ID NO: 32. In some embodiments, the polypeptide comprises an Fc domain having an amino acid sequence with at least 88% identity to SEQ ID NO: 32. In some embodiments, the polypeptide comprises an Fc domain having an amino acid sequence with at least 90% identity to SEQ ID NO: 32. In some embodiments, the polypeptide comprises an Fc domain having an amino acid sequence with at least 92% identity to SEQ ID NO: 32. In some embodiments, the polypeptide comprises an Fc domain having an amino acid sequence with at least 95% identity to SEQ ID NO: 32. In some embodiments, the polypeptide comprises an Fc domain having an amino acid sequence with at least 96% identity to SEQ ID NO: 32. In some embodiments, the polypeptide comprises an Fc domain having an amino acid sequence with at least 97% identity to SEQ ID NO: 32. In some embodiments, the polypeptide comprises an Fc domain having an amino acid sequence with at least 98% identity to SEQ ID NO: 32. In some embodiments, the polypeptide comprises an Fc domain having an amino acid sequence with at least 99% identity to SEQ ID NO: 32.

In some embodiments, the Fc domain comprises the amino acid sequence of SEQ ID: 33.

(SEQ ID NO: 33)
ESKYGPPCPPCPAPEVEGGPSVFLFPPKPKDTLMISRTPEVTCVVVGVS
QEDPEVQFNWYVDGVEVHNAKTKPREEQFNSTYRVVSVLQVLHVDWLNG
KEYKCKVSNKGLPSSIEKTISKAKGQPREPQVYTLPPSQEEMTKNQVSL
TCLVKGFYPSDIVVEWESNGQPENNYKTTPPVLDSDGSFFLYSRLTVDK
SRWQEGNVFSCSVMHEALHNHYTQKSLSLSLG

In some embodiments, the polypeptide comprises an Fc domain having an amino acid sequence with at least 80% identity to SEQ ID NO: 33. In some embodiments, the polypeptide comprises an Fc domain having an amino acid sequence with at least 82% identity to SEQ ID NO: 33. In some embodiments, the polypeptide comprises an Fc domain having an amino acid sequence with at least 85% identity to SEQ ID NO: 33. In some embodiments, the polypeptide comprises an Fc domain having an amino acid sequence with at least 88% identity to SEQ ID NO: 33. In some embodiments, the polypeptide comprises an Fc domain having an amino acid sequence with at least 90% identity to SEQ ID NO: 33. In some embodiments, the polypeptide comprises an Fc domain having an amino acid sequence with at least 92% identity to SEQ ID NO: 33. In some embodiments, the polypeptide comprises an Fc domain having an amino acid sequence with at least 95% identity to SEQ ID NO: 33. In some embodiments, the polypeptide comprises an Fc domain having an amino acid sequence with at least 96% identity to SEQ ID NO: 33. In some embodiments, the polypeptide comprises an Fc domain having an amino acid sequence with at least 97% identity to SEQ ID NO: 33. In some embodiments, the polypeptide comprises an Fc domain having an amino acid sequence with at least 98% identity to SEQ ID NO: 33. In some embodiments, the polypeptide comprises an Fc domain having an amino acid sequence with at least 99% identity to SEQ ID NO: 33.

In some embodiments, the Fc domain comprises the amino acid sequence of SEQ ID: 34.

(SEQ ID NO: 34)
DKTHTCPPCPAPEVEGGPSVFLFPPKPKDTLMISRTPEVTCVVVGVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG

In some embodiments, the polypeptide comprises an Fc domain having an amino acid sequence with at least 80% identity to SEQ ID NO: 34. In some embodiments, the polypeptide comprises an Fc domain having an amino acid sequence with at least 82% identity to SEQ ID NO: 34. In some embodiments, the polypeptide comprises an Fc domain having an amino acid sequence with at least 85% identity to SEQ ID NO: 34. In some embodiments, the polypeptide comprises an Fc domain having an amino acid sequence with at least 88% identity to SEQ ID NO: 34. In some embodiments, the polypeptide comprises an Fc domain having an amino acid sequence with at least 90% identity to SEQ ID NO: 34. In some embodiments, the polypeptide comprises an Fc domain having an amino acid sequence with at least 92% identity to SEQ ID NO: 34. In some embodiments, the polypeptide comprises an Fc domain having an amino acid sequence with at least 95% identity to SEQ ID NO: 34. In some embodiments, the polypeptide comprises an Fc domain having an amino acid sequence with at least 96% identity to SEQ ID NO: 34. In some embodiments, the polypeptide comprises an Fc domain having an amino acid sequence with at least 97% identity to SEQ ID NO: 34. In some embodiments, the polypeptide comprises an Fc domain having an amino acid sequence with at least 98% identity to SEQ ID NO: 34. In some embodiments, the polypeptide comprises an Fc domain having an amino acid sequence with at least 99% identity to SEQ ID NO: 34.

In some embodiments, the Fc domain comprises the amino acid sequence of SEQ ID: 35.

(SEQ ID NO: 35)
DKTHTCPPCPAPELLGGPSVFLFPPKPKDTLMISRTPEVTCVVVDVSHE
DPEVKFNWYVDGVEVHNAKTKPREEQYNSTYRVVSVLQVLHVDWLNGKE
YKCKVSNKALPAPIEKTISKAKGQPREPQVYTLPPSREEMTKNQVSLTC
LVKGFYPSDIVVEWESNGQPENNYKTTPPVLDSDGSFFLYSKLTVDKSR
WQQGNVFSCSVMHEALHNHYTQKSLSLSPG

In some embodiments, the polypeptide comprises an Fc domain having an amino acid sequence with at least 80% identity to SEQ ID NO: 35. In some embodiments, the polypeptide comprises an Fc domain having an amino acid sequence with at least 82% identity to SEQ ID NO: 35. In some embodiments, the polypeptide comprises an Fc domain having an amino acid sequence with at least 85% identity to SEQ ID NO: 35. In some embodiments, the polypeptide comprises an Fc domain having an amino acid sequence with at least 88% identity to SEQ ID NO: 35. In some embodiments, the polypeptide comprises an Fc domain having an amino acid sequence with at least 90% identity to SEQ ID NO: 35. In some embodiments, the polypeptide comprises an Fc domain having an amino acid sequence with at least 92% identity to SEQ ID NO: 35. In some embodiments, the polypeptide comprises an Fc domain having an amino acid sequence with at least 95% identity to SEQ ID NO: 35. In some embodiments, the polypeptide comprises an Fc domain having an amino acid sequence with at least 96% identity to SEQ ID NO: 35. In some embodiments, the polypeptide comprises an Fc domain having an amino acid sequence with at least 97% identity to SEQ ID NO: 35. In some embodiments, the polypeptide comprises an Fc domain having an amino acid sequence with at least 98% identity to SEQ ID NO: 35. In some embodiments, the polypeptide comprises an Fc domain having an amino acid sequence with at least 99% identity to SEQ ID NO: 35.

In some embodiments, the Fc domain comprises the amino acid sequence of SEQ ID: 36.

(SEQ ID NO: 36)
DKTHTCPPCPAPEVEGGPSVFLFPPKPKDTLMISRTPEVTCVVVGVSHE
DPEVKFNWYVDGVEVHNAKTKPREEQYNSTYRVVSVLQVLHVDWLNGKE
YKCKVSNKALPAPIEKTISKAKGQPREPQVYTLPPSREEMTKNQVSLTC
LVKGFYPSDIVVEWESNGQPENNYKTTPPVLDSDGSFFLYSKLTVDKSR
WQQGNVFSCSVMHEALHNHYTQKSLSLSPG

In some embodiments, the polypeptide comprises an Fc domain having an amino acid sequence with at least 80% identity to SEQ ID NO: 36. In some embodiments, the polypeptide comprises an Fc domain having an amino acid sequence with at least 82% identity to SEQ ID NO: 36. In some embodiments, the polypeptide comprises an Fc domain having an amino acid sequence with at least 85% identity to SEQ ID NO: 36. In some embodiments, the polypeptide comprises an Fc domain having an amino acid sequence with at least 88% identity to SEQ ID NO: 36. In some embodiments, the polypeptide comprises an Fc domain having an amino acid sequence with at least 90% identity to SEQ ID NO: 36. In some embodiments, the polypeptide comprises an Fc domain having an amino acid sequence with at least 92% identity to SEQ ID NO: 36. In some embodiments, the polypeptide comprises an Fc domain having an amino acid sequence with at least 95% identity to SEQ ID NO: 36. In some embodiments, the polypeptide comprises an Fc domain having an amino acid sequence with at least 96% identity to SEQ ID NO: 36. In some embodiments, the polypeptide comprises an Fc domain having an amino acid sequence with at least 97% identity to SEQ ID NO: 36. In some embodiments, the polypeptide comprises an Fc domain having an amino acid sequence with at least 98% identity to SEQ ID NO: 36. In some embodiments, the polypeptide comprises an Fc domain having an amino acid sequence with at least 99% identity to SEQ ID NO: 36.

In some embodiments, an anti-C3b VHH is fused to an Fc domain. In some embodiments, the polypeptide comprises, from the N-terminus to the C-terminus, an Fc domain and a moiety comprising a variable domain capable of binding to complement C3b, or a variant thereof.

In some embodiments, an anti-C3b antibody or antigen binding fragment thereof comprises a VHH-Fc domain fusion of

(SEQ ID NO: 1)
EVQLLESGGGLVQPGGSLRLSCAASGRTFSNYHMGWFRQAPGQGREFVA
TIIRTGETIYYADSVKGRFTISRDNSKNTLYLQMNSLRAEDTAVYYCAA
ATSGWNIKTVTPYEYWGQGTLVTVSSESKYGPPCPPCPAPEFLGGPSVF
LFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYT
QKSLSLSLGK.

In some embodiments, an anti-C3b antibody or antigen binding fragment thereof comprises a VHH-Fc domain fusion of

(SEQ ID NO: 2)
EVQLLESGGGLVQPGGSLRLSCAASGRTFSNYHMGWFRQAPGQGREFVA
TIIRHGTTIYYADSVKGRFTISRDNSKNTLYLQMNSLRAEDTAVYYCAA
ATSGWHIKTVTPYEYWGQGTLVTVSSESKYGPPCPPCPAPEVEGGPSVF
LFPPKPKDTLMISRTPEVTCVVVGVSQEDPEVQFNWYVDGVEVHNAKTK
PREEQFNSTYRVVSVLQVLHVDWLNGKEYKCKVSNKGLPSSIEKTISKA
KGQPREPQVYTLPPSQEEMTKNQVSLTCLVKGFYPSDIVVEWESNGQPE
NNYKTTPPVLDSDGSFFLYSRLTVDKSRWQEGNVFSCSVMHEALHNHYT
QKSLSLSLGK.

In some embodiments, the polypeptide that is capable of binding to C3b comprises an amino acid sequence of MCP, and a VHH-Fc domain fusion of SEQ ID NO: 1. In some embodiments, the polypeptide that is capable of binding to C3b comprises an amino acid sequence of MCP, and a VHH-Fc domain fusion that is at least 80% identical to SEQ ID NO: 1. In some embodiments, the polypeptide that is capable of binding to C3b comprises an amino acid sequence of MCP, and a VHH-Fc domain fusion that is at least 85% identical to SEQ ID NO: 1. In some embodiments, the polypeptide that is capable of binding to C3b comprises an amino acid sequence of MCP, and a VHH-Fc domain fusion that is at least 88% identical to SEQ ID NO: 1. In some embodiments, the polypeptide that is capable of binding to C3b comprises an amino acid sequence of MCP, and a VHH-Fc domain fusion that is at least 90% identical to SEQ ID NO: 1. In some embodiments, the polypeptide that is capable of binding to C3b comprises an amino acid sequence of MCP, and a VHH-Fc domain fusion that is at least 93% identical to SEQ ID NO: 1. In some embodiments, the polypeptide that is capable of binding to C3b comprises an amino acid sequence of MCP, and a VHH-Fc domain fusion that is at least 95% identical to SEQ ID NO: 1. In some embodiments, the polypeptide that is capable of binding to C3b comprises an amino acid sequence of MCP, and a VHH-Fc domain fusion that is at least 96% identical to SEQ ID NO: 1. In some embodiments, the polypeptide that is capable of binding to C3b comprises an amino acid sequence of MCP, and a VHH-Fc domain fusion that is at least 97% identical to SEQ ID NO: 1. In some embodiments, the polypeptide that is capable of binding to C3b comprises an amino acid sequence of MCP, and a VHH-Fc domain fusion that is at least 98% identical to SEQ ID NO: 1. In some embodiments, the polypeptide that is capable of binding to C3b comprises an amino acid sequence of MCP, and a VHH-Fc domain fusion that is at least 99% identical to SEQ ID NO: 1. In some embodiments, the polypeptide that is capable of binding to C3b comprises an amino acid sequence of MCP, and a VHH-Fc domain fusion that is at least 99% identical to SEQ ID NO: 1.

In some embodiments, the polypeptide that is capable of binding to C3b comprises an amino acid sequence of MCP, and a VHH-Fc domain fusion of SEQ ID NO: 2. In some embodiments, the polypeptide that is capable of binding to C3b comprises an amino acid sequence of MCP, and a VHH-Fc domain fusion that is at least 80% identical to SEQ ID NO: 2. In some embodiments, the polypeptide that is capable of binding to C3b comprises an amino acid sequence of MCP, and a VHH-Fc domain fusion that is at least 85% identical to SEQ ID NO: 2. In some embodiments, the polypeptide that is capable of binding to C3b comprises an amino acid sequence of MCP, and a VHH-Fc domain fusion that is at least 88% identical to SEQ ID NO: 12 In some embodiments, the polypeptide that is capable of binding to C3b comprises an amino acid sequence of MCP, and a VHH-Fc domain fusion that is at least 90% identical to SEQ ID NO: 2. In some embodiments, the polypeptide that is capable of binding to C3b comprises an amino acid sequence of MCP, and a VHH-Fc domain fusion that is at least 93% identical to SEQ ID NO: 2. In some embodiments, the polypeptide that is capable of binding to C3b comprises an amino acid sequence of MCP, and a VHH-Fc domain fusion that is at least 95% identical to SEQ ID NO: 2. In some embodiments, the polypeptide that is capable of binding to C3b comprises an amino acid sequence of MCP, and a VHH-Fc domain fusion that is at least 96% identical to SEQ ID NO: 2. In some embodiments, the polypeptide that is capable of binding to C3b comprises an amino acid sequence of MCP, and a VHH-Fc domain fusion that is at least 97% identical to SEQ ID NO: 2. In some embodiments, the polypeptide that is capable of binding to C3b comprises an amino acid sequence of MCP, and a VHH-Fc domain fusion that is at least 98% identical to SEQ ID NO: 2. In some embodiments, the polypeptide that is capable of binding to C3b comprises an amino acid sequence of MCP, and a VHH-Fc domain fusion that is at least 99% identical to SEQ ID NO: 2. In some embodiments, the polypeptide that is capable of binding to C3b comprises an amino acid sequence of MCP, and a VHH-Fc domain fusion that is at least 99% identical to SEQ ID NO: 2.

### Linker

The linker may be a flexible peptide sequence, such as (GGGGS)n, where n is an integer from 1 to 10. Alternatively, the linker may be a cleavable linker, such as sequences cleavable by specific enzymes (e.g., proteases), allowing for controlled release of the therapeutic protein at the target site. In another aspect, the linker may be a rigid linker, such as alpha-helical peptides or proline-rich sequences, to maintain a fixed orientation between domains. The linker can also incorporate glycosylation sites to improve solubility or cysteine residues for disulfide bond formation, enhancing structural stability. The length and composition of the linker can be optimized to ensure minimal steric hindrance, preserve the biological function of the fusion protein, or to enhance pharmacokinetic properties.

In some embodiments, a polypeptide capable of binding to C3b comprises a first moiety linked to a second moiety. In some embodiments, the first moiety and the second moiety are linked via a linker (Linker 1).

In some embodiments, a linker between the first moiety and the second moiety is between 3 and 20 amino acids. In some embodiments, a linker between the first moiety and the second moiety is between 5 and 15 amino acids. In some embodiments, a linker between the first moiety and the second moiety is between 10 and 15 amino acids.

In some embodiments, a linker between the first moiety and the second moiety is 3 amino acids. In some embodiments, a linker between the first moiety and the second moiety is 4 amino acids. In some embodiments, a linker between the first moiety and the second moiety is 5 amino acids. In some embodiments, a linker between the first moiety and the second moiety is 6 amino acids. In some embodiments, a linker between the first moiety and the second moiety is 7 amino acids. In some embodiments, a linker between the first moiety and the second moiety is 8 amino acids. In some embodiments, a linker between the first moiety and the second moiety is 9 amino acids. In some embodiments, a linker between the first moiety and the second moiety is 10 amino acids. In some embodiments, a linker between the first moiety and the second moiety is 11 amino acids. In some embodiments, a linker between the first moiety and the second moiety is 12 amino acids. In some embodiments, a linker between the first moiety and the second moiety is 13 amino acids. In some embodiments, a linker between the first moiety and the second moiety is 14 amino acids. In some embodiments, a linker between the first moiety and the second moiety is 15 amino acids. In some embodiments, a linker between the first moiety and the second moiety is 16 amino acids. In some embodiments, a linker between the first moiety and the second moiety is 17 amino acids. In some embodiments, a linker between the first moiety and the second moiety is 18 amino acids. In some embodiments, a linker between the first moiety and the second moiety is 19 amino acids. In some embodiments, a linker between the first moiety and the second moiety is 20 amino acids. In some embodiments, a linker between the first moiety and the second moiety is 21 amino acids. In some embodiments, a linker between the first moiety and the second moiety is 22 amino acids. In some embodiments, a linker between the first moiety and the second moiety is 23 amino acids. In some embodiments, a linker between the first moiety and the second moiety is 24 amino acids. In some embodiments, a linker between the first moiety and the second moiety is 25 amino acids.

In some embodiments, a linker between the first moiety and the second moiety comprises GGGGS (SEQ ID NO: 37). In some embodiments, a linker between the first moiety and the second moiety comprises GGGGSGGGGS (SEQ ID NO: 38). In some embodiments, a linker between the first moiety and the second moiety comprises GGGGSGGGGSGGGGS (SEQ ID NO: 39). In some embodiments, a linker between the first moiety and the second moiety comprises GGGGSGGGGSGGGGSGGGGS (SEQ ID NO: 40).

In some embodiments, a linker between the first moiety and the second moiety consists of glycine residues. In some embodiments, a linker between the first moiety and the second moiety comprises of glycine, proline and serine residues.

In some embodiments, a polypeptide capable of binding to C3b comprises a first moiety linked to a third moiety. In some embodiments, the first moiety and the third moiety are linked via a linker (Linker 2). In some embodiments, a polypeptide capable of binding to C3b comprises a second moiety linked to a third moiety. In some embodiments, the second moiety and the third moiety are linked via a linker (Linker 3).

In some embodiments, a linker between the first moiety and the third moiety is between 3 and 20 amino acids. In some embodiments, a linker between the first moiety and the third moiety is between 5 and 15 amino acids. In some embodiments, a linker between the first moiety and the third moiety is between 5 and 10 amino acids. In some embodiments, a linker between the first moiety and the third moiety is between 10 and 15 amino acids.

In some embodiments, a linker between the first moiety and the third moiety is 3 amino acids. In some embodiments, a linker between the first moiety and the third moiety is 4 amino acids. In some embodiments, a linker between the first moiety and the third moiety is 5 amino acids. In some embodiments, a linker between the first moiety and the third moiety is 6 amino acids. In some embodiments, a linker between the first moiety and the third moiety is 7 amino acids. In some embodiments, a linker between the first moiety and the third moiety is 8 amino acids. In some embodiments, a linker between the first moiety and the third moiety is 9 amino acids. In some embodiments, a linker between the first moiety and the third moiety is 10 amino acids. In some embodiments, a linker between the first moiety and the third moiety is 11 amino acids. In some embodiments, a linker between the first moiety and the third moiety is 12 amino acids. In some embodiments, a linker between the first moiety and the third moiety is 13 amino acids. In some embodiments, a linker between the first moiety and the third moiety is 14 amino acids. In some embodiments, a linker between the first moiety and the third moiety is 15 amino acids. In some embodiments, a linker between the first moiety and the third moiety is 16 amino acids. In some embodiments, a linker between the first moiety and the third moiety is 17 amino acids. In some embodiments, a linker between the first moiety and the third moiety is 18 amino acids. In some embodiments, a linker between the first moiety and the third moiety is 19 amino acids. In some embodiments, a linker between the first moiety and the third moiety is 20 amino acids. In some embodiments, a linker between the first moiety and the third moiety is 21 amino acids. In some embodiments, a linker between the first moiety and the third moiety is 22 amino acids. In some embodiments, a linker between the first moiety and the third moiety is 23 amino acids. In some embodiments, a linker between the first moiety and the third moiety is 24 amino acids. In some embodiments, a linker between the first moiety and the third moiety is 25 amino acids.

In some embodiments, a linker between the first moiety and the third moiety comprises GGGGGS (SEQ ID NO: 41). In some embodiments, a linker between the first moiety and the third moiety comprises GGGGGSGGGGGS (SEQ ID NO: 42).

In some embodiments, a linker between the first moiety and the third moiety consists of glycine residues. In some embodiments, a linker between the first moiety and the third moiety comprises of glycine, proline and serine residues.

In some embodiments, a linker between the second moiety and the third moiety is between 3 and 20 amino acids. In some embodiments, a linker between the second moiety and the third moiety is between 5 and 15 amino acids. In some embodiments, a linker between the second moiety and the third moiety is between 10 and 15 amino acids.

In some embodiments, a linker between the second moiety and the third moiety is 3 amino acids. In some embodiments, a linker between the second moiety and the third moiety is 4 amino acids. In some embodiments, a linker between the second moiety and the third moiety is 5 amino acids. In some embodiments, a linker between the second moiety and the third moiety is 6 amino acids. In some embodiments, a linker between the second moiety and the third moiety is 7 amino acids. In some embodiments, a linker between the second moiety and the third moiety is 8 amino acids. In some embodiments, a linker between the second moiety and the third moiety is 9 amino acids. In some embodiments, a linker between the second moiety and the third moiety is 10 amino acids. In some embodiments, a linker between the second moiety and the third moiety is 11 amino acids. In some embodiments, a linker between the second moiety and the third moiety is 12 amino acids. In some embodiments, a linker between the second moiety and the third moiety is 13 amino acids. In some embodiments, a linker between the second moiety and the third moiety is 14 amino acids. In some embodiments, a linker between the second moiety and the third moiety is 15 amino acids. In some embodiments, a linker between the second moiety and the third moiety is 16 amino acids. In some embodiments, a linker between the second moiety and the third moiety is 17 amino acids. In some embodiments, a linker between the second moiety and the third moiety is 18 amino acids. In some embodiments, a linker between the second moiety and the third moiety is 19 amino acids. In some embodiments, a linker between the second moiety and the third moiety is 20 amino acids. In some embodiments, a linker between the second moiety and the third moiety is 21 amino acids. In some embodiments, a linker between the second moiety and the third moiety is 22 amino acids. In some embodiments, a linker between the second moiety and the third moiety is 23 amino acids. In some embodiments, a linker between the second moiety and the third moiety is 24 amino acids. In some embodiments, a linker between the second moiety and the third moiety is 25 amino acids.

In some embodiments, a linker between the second moiety and the third moiety comprises GGGGGS (SEQ ID NO: 41). In some embodiments, a linker between the second moiety and the third moiety comprises GGGGGSGGGGGS (SEQ ID NO: 42).

In some embodiments, a linker between the second moiety and the third moiety consists of glycine residues. In some embodiments, a linker between the second moiety and the third moiety comprises of glycine, proline and serine residues.

### Exemplary Polypeptides and Proteins

In one aspect, the present disclosure provides, among other things a polypeptide that is capable of binding to C3b, wherein the polypeptide comprises (A) a first moiety and (B) a second moiety, wherein the first moiety comprises a variable domain capable of binding to C3b, and the second moiety comprises an amino acid sequence of MCP or a variant thereof.

In one aspect, the present disclosure provides, among other things a polypeptide that is capable of binding to C3b, wherein the polypeptide comprises (A) a first moiety, (B) a second moiety, and (C) a third moiety, wherein the first moiety comprises a variable domain capable of binding to C3b, the second moiety comprises an amino acid sequence of MCP or a variant thereof, and the third moiety comprises a half-life extension domain.

In some embodiments, the polypeptide that is capable of binding to C3b comprises a format and components shown in Table C.

TABLE C
Exemplary Polypeptides Capable of Binding to C3b

| Construct | First Moiety | Second Moiety | Third Moiety | Format |
|---|---|---|---|---|
| AP1 | anti-C3b-VHH | MCP domain | IgG4 Fc domain (F234V, | Fc-VHH-MCP |
| | | D2-4 | L235E D265G, and S228P) | |
| AP2 | anti-C3b-VHH | MCP domain | IgG4 Fc domain (T307Q, | Fc-VHH-MCP |
| | | D2-4 | Q311V, A378V, F234V, | |
| | | | L235E, D265G, and S228P) | |
| AP3 | anti-C3b-VHH | MCP domain | | VHH-MCP |
| | | D2-4 | | |
| AP4 | anti-C3b-VHH | MCP domain | IgG1 Fc domain 9 T307Q, | Fc-VHH-MCP |
| | | D2-4 | Q311V, A378V, F234V, | |
| | | | L235E, and D265G) | |
| AP5 | anti-C3b-VHH | MCP domain | IgG4 Fc domain (T307Q, | Fc-VHH-MCP |
| | | D2-4(S177R) | Q311V, A378V, F234V, | |
| | | | L235E, D265G, and S228P) | |
| AP6 | anti-C3b-VHH | MCP domain | — | VHH-MCP |
| | | D2-4 | | |
| AP7 | anti-C3b-VHH | MCP domain | IgG4 Fc domain (T307Q, | VHH-MCP-Fc |
| | | D2-4 | Q311V, A378V, F234V, | |
| | | | L235E, D265G, and S228P) | |
| AP8 | anti-C3b-VHH | MCP domain | IgG4 Fc domain (S228P) | VHH-MCP-Fc |
| | | D2-4 | | |
| AP9 | anti-C3b-VHH | MCP domain | IgG4 Fc domain (S228P) | Fc-VHH-MCP |
| | | D2-4 | | |
| AP10 | anti-C3b-VHH | MCP domain | IgG4 Fc domain 9 T307Q, | VHH-MCP-Fc |
| | | D2-4 (10aa) | Q311V, A378V, F234V, | |
| | | | L235E, D265G, and S228P) | |
| AP11 | anti-C3b-VHH | MCP domain | IgG4 Fc domain (T307Q, | VHH-MCP-Fc |
| | | D2-4 (5aa) | Q311V, A378V, F234V, | |
| | | | L235E, D265G, and S228P) | |
| AP12 | anti-C3b-VHH | MCP domain | IgG4 Fc domain (F234V, | Fc-VHH-MCP |
| | | D2-4 | L235E D265G, and S228P) | |
| AP13 | anti-C3b-VHH | MCP domain | IgG4 Fc domain (T307Q, | Fc-VHH-MCP |
| | | D2-4 | Q311V, A378V, F234V, | |
| | | | L235E, D265G, and S228P) | |
| AP14 | anti-C3b-VHH | MCP domain | IgG4 Fc domain (S228P) | Fc-VHH-MCP |
| | | D2-4 | | |
| AP15 | anti-C3b-VHH | MCP domain | IgG4 Fc domain (T307Q, | VHH-MCP-Fc |
| | | D2-4 | Q311V, A378V, F234V, | |
| | | | L235E, D265G, and S228P) | |
| AP16 | anti-C3b-VHH | MCP domain | IgG4 Fc domain (F234V, | Fc-VHH-MCP |
| | | D2-4 | L235E D265G, and S228P) | |
| AP17 | anti-C3b-VHH | MCP domain | IgG4 Fc domain (F234V, | Fc-VHH-MCP |
| | | D2-4 | L235E D265G, and S228P) | |
| Ap18 | anti-C3b-VHH | MCP domain | IgG4 Fc domain (F234V, | Fc-VHH-MCP |
| | | D2-4 | L235E D265G, and S228P) | |

In some embodiments, the first moiety comprises an anti-C3b VHH.

In some embodiments, the second moiety comprises MCP.

In some embodiments, the second moiety comprises domains 2-4 of MCP. In some embodiments, the second moiety comprises a sequence corresponding to amino acids residues 96-285 of MCP, according to SEQ ID NO: 14.

In some embodiments, the third moiety comprises an Fc domain.

In some embodiments, the third moiety comprises an Fc domain derived from IgG4. In some embodiments, the second moiety comprises an Fc domain from IgG1.

In some embodiments, the polypeptide comprises from the N-terminus to the C-terminus, the first moiety comprising a variable domain capable of binding to complement C3b, a linker, and a second moiety comprising an amino acid sequence of a membrane complement protein (MCP), or a variant thereof.

In some embodiments, the polypeptide comprises, from the N-terminus to the C-terminus, the first moiety comprising an anti-C3bVHH and the second moiety comprising the domains 2-4 of MCP.

In some embodiments, the polypeptide comprises, from the N-terminus to the C-terminus, the variable domain of the first moiety comprising a HCDR1 of SEQ ID NO: 16, a HCDR2 of SEQ ID NO: 17, and a HCDR3 of SEQ ID NO: 18, and the second moiety comprising the domains 2-4 of MCP.

In some embodiments, the polypeptide comprises, from the N-terminus to the C-terminus, the variable domain of the first moiety comprising a HCDR1 of SEQ ID NO: 19, a HCDR2 of SEQ ID NO: 20, and a HCDR3 of SEQ ID NO: 18, and the second moiety comprising the domains 2-4 of MCP.

In some embodiments, the polypeptide comprises, from the N-terminus to the C-terminus, the variable domain of the first moiety comprising a HCDR1 of SEQ ID NO: 16, a HCDR2 of SEQ ID NO: 26, and a HCDR3 of SEQ ID NO: 22, and the second moiety comprising the domains 2-4 of MCP.

In some embodiments, the polypeptide comprises, from the N-terminus to the C-terminus, the variable domain of the first moiety comprising a HCDR1 of SEQ ID NO: 19, a HCDR2 of SEQ ID NO: 27, and a HCDR3 of SEQ ID NO: 22, and the second moiety comprising the domains 2-4 of MCP.

In some embodiments, the polypeptide comprises, from the N-terminus to the C-terminus, the first moiety comprising a variable domain having the sequence of SEQ ID NO: 10 and the second moiety comprising the amino acid sequence of SEQ ID NO: 9.

In some embodiments, the polypeptide comprises, from the N-terminus to the C-terminus, the first moiety comprising a variable domain having the sequence of SEQ ID NO: 13 and the second moiety comprising the amino acid sequence of SEQ ID NO: 9.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 2. In some embodiments, the polypeptide comprises an amino acid sequence at least 80% identical to SEQ ID NO: 2. In some embodiments, the polypeptide comprises an amino acid sequence at least 85% identical to SEQ ID NO: 2. In some embodiments, the polypeptide comprises an amino acid sequence at least 88% identical to SEQ ID NO: 2. In some embodiments, the polypeptide comprises an amino acid sequence at least 90% identical to SEQ ID NO: 2. In some embodiments, the polypeptide comprises an amino acid sequence at least 91% identical to SEQ ID NO: 2. In some embodiments, the polypeptide comprises an amino acid sequence at least 92% identical to SEQ ID NO: 2. In some embodiments, the polypeptide comprises an amino acid sequence at least 93% identical to SEQ ID NO: 2. In some embodiments, the polypeptide comprises an amino acid sequence at least 94% identical to SEQ ID NO: 2. In some embodiments, the polypeptide comprises an amino acid sequence at least 95% identical to SEQ ID NO: 2. In some embodiments, the polypeptide comprises an amino acid sequence at least 96% identical to SEQ ID NO: 2. In some embodiments, the polypeptide comprises an amino acid sequence at least 97% identical to SEQ ID NO: 2. In some embodiments, the polypeptide comprises an amino acid sequence at least 98% identical to SEQ ID NO: 2. In some embodiments, the polypeptide comprises an amino acid sequence at least 99% identical to SEQ ID NO: 2.

In some embodiments, the polypeptide comprises the amino acid sequence of

(SEQ ID NO: 8)
EVQLLESGGGLVQPGGSLRLSCAASGRTFSNYHMGWFRQAPGQGREFVA
TIIRHGTTIYYADSVKGRFTISRDNSKNTLYLQMNSLRAEDTAVYYCAA
ATSGWHIKTVTPYEYWGQGTLVTVSSGGGGSGGGGSGGGGSRETCPYIR
DPLNGQAVPANGTYEFGYQMHFICNEGYYLIGEEILYCELKGSVAIWSG
KPPICEKVLCTPPPKIKNGKHTFSEVEVFEYLDAVTYSCDPAPGPDPFS
LIGESTIYCGDNSVWSRAAPECKVVKCRFPVVENGKQISGFGKKFYYKA
TVMFECDKGFYLDGSDTIVCDSNSTWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 8. In some embodiments, the polypeptide comprises an amino acid sequence at least 80% identical to SEQ ID NO: 8. In some embodiments, the polypeptide comprises an amino acid sequence at least 85% identical to SEQ ID NO: 8. In some embodiments, the polypeptide comprises an amino acid sequence at least 88% identical to SEQ ID NO: 8. In some embodiments, the polypeptide comprises an amino acid sequence at least 90% identical to SEQ ID NO: 8. In some embodiments, the polypeptide comprises an amino acid sequence at least 91% identical to SEQ ID NO: 8. In some embodiments, the polypeptide comprises an amino acid sequence at least 92% identical to SEQ ID NO: 8. In some embodiments, the polypeptide comprises an amino acid sequence at least 93% identical to SEQ ID NO: 8. In some embodiments, the polypeptide comprises an amino acid sequence at least 94% identical to SEQ ID NO: 8. In some embodiments, the polypeptide comprises an amino acid sequence at least 95% identical to SEQ ID NO: 8. In some embodiments, the polypeptide comprises an amino acid sequence at least 96% identical to SEQ ID NO: 8. In some embodiments, the polypeptide comprises an amino acid sequence at least 97% identical to SEQ ID NO: 8. In some embodiments, the polypeptide comprises an amino acid sequence at least 98% identical to SEQ ID NO: 8. In some embodiments, the polypeptide comprises an amino acid sequence at least 99% identical to SEQ ID NO: 8.

In some embodiments, the polypeptide comprises the amino acid sequence of

(SEQ ID NO: 5)
EVQLLESGGGLVQPGGSLRLSCAASGRTFSNYHMGWFRQAPGQGREFVA
TIIRTGETIYYADSVKGRFTISRDNSKNTLYLQMNSLRAEDTAVYYCAA
ATSGWNIKTVTPYEYWGQGTLVTVSSGGGGSGGGGSGGGGSRETCPYIR
DPLNGQAVPANGTYEFGYQMHFICNEGYYLIGEEILYCELKGSVAIWSG
KPPICEKVLCTPPPKIKNGKHTFSEVEVFEYLDAVTYSCDPAPGPDPFS
LIGESTIYCGDNSVWSRAAPECKVVKCRFPVVENGKQISGFGKKFYYKA
TVMFECDKGFYLDGSDTIVCDSNSTWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 5. In some embodiments, the polypeptide comprises an amino acid sequence at least 80% identical to SEQ ID NO: 5. In some embodiments, the polypeptide comprises an amino acid sequence at least 85% identical to SEQ ID NO: 5. In some embodiments, the polypeptide comprises an amino acid sequence at least 88% identical to SEQ ID NO: 5. In some embodiments, the polypeptide comprises an amino acid sequence at least 90% identical to SEQ ID NO: 5. In some embodiments, the polypeptide comprises an amino acid sequence at least 91% identical to SEQ ID NO: 5. In some embodiments, the polypeptide comprises an amino acid sequence at least 92% identical to SEQ ID NO: 5. In some embodiments, the polypeptide comprises an amino acid sequence at least 93% identical to SEQ ID NO: 5. In some embodiments, the polypeptide comprises an amino acid sequence at least 94% identical to SEQ ID NO: 5. In some embodiments, the polypeptide comprises an amino acid sequence at least 95% identical to SEQ ID NO: 5. In some embodiments, the polypeptide comprises an amino acid sequence at least 96% identical to SEQ ID NO: 5. In some embodiments, the polypeptide comprises an amino acid sequence at least 97% identical to SEQ ID NO: 5. In some embodiments, the polypeptide comprises an amino acid sequence at least 98% identical to SEQ ID NO: 5. In some embodiments, the polypeptide comprises an amino acid sequence at least 99% identical to SEQ ID NO: 5.

In some embodiments, the polypeptide comprises, from the N-terminus to the C-terminus, the third moiety comprising an Fc domain, the first moiety comprising an anti-C3bVHH and the second moiety comprising the domains 2-4 of MCP.

In some embodiments, the polypeptide comprises from the N-terminus to the C-terminus, the third moiety comprising the Fc domain having the amino acid modifications of a)T307Q, Q311V and A378V; b) F234V, L235E and D265G; and c) S228P, the first moiety comprising the sequence of SEQ ID NO: 10, and the second moiety comprising the sequence of SEQ ID NO: 9.

In some embodiments, the polypeptide comprises, from the N-terminus to the C-terminus, the third moiety comprising the Fc domain having the amino acid modifications of a)T307Q, Q311V and A378V; and b) F234V, L235E and D265G, the first moiety comprising the sequence of SEQ ID NO: 10, and the second moiety comprising the sequence of SEQ ID NO: 9.

In some embodiments, the polypeptide comprises, from the N-terminus to the C-terminus, the third moiety comprising the Fc domain having the amino acid modifications of a) T307Q, Q311V and A378V; b) F234V, L235E and D265G; and c) S228P, the first moiety comprising the sequence of SEQ ID NO: 10, the second moiety comprising the sequence of SEQ ID NO: 15.

In some embodiments, the polypeptide comprises, from the N-terminus to the C-terminus, the third moiety comprising the Fc domain having the amino acid modifications of a) F234V, L235E and D265G, and b) S228P, the first moiety comprising the sequence of SEQ ID NO: 10, the second moiety comprising the sequence of SEQ ID NO: 9.

In some embodiments, the polypeptide comprises, from the N-terminus to the C-terminus, the third moiety comprising the Fc domain having the amino acid modification of S228P, the first moiety comprising the sequence of SEQ ID NO: 13, and the second moiety comprising the sequence of SEQ ID NO: 9.

In some embodiments, the polypeptide comprises, from the N-terminus to the C-terminus, the third moiety comprising the Fc domain having the amino acid modifications of a) F234V, L235E and D265G and b) S228P, the first moiety comprising the sequence of SEQ ID NO: 13, and the second moiety comprising the sequence of SEQ ID NO: 9.

In some embodiments, the polypeptide comprises, from the N-terminus to the C-terminus, the third moiety comprising the Fc domain having the amino acid modifications of a)T307Q, Q311V and A378V; b) F234V, L235E and D265G; and c) S228P, the first moiety comprising the sequence of SEQ ID NO: 13, and the second moiety comprising the sequence of SEQ ID NO: 9.

In some embodiments, the polypeptide comprises, from the N-terminus to the C-terminus, the third moiety comprising the Fc domain having the amino acid modification of S228P, the first moiety comprising the sequence of SEQ ID NO: 10, and the second moiety comprising the sequence of SEQ ID NO: 9.

In some embodiments, the polypeptide comprises, from the N-terminus to the C-terminus, the third moiety comprising the Fc domain having the amino acid modifications of a) F234V, L235E and D265G and b) S228P, the first moiety comprising the sequence of SEQ ID NO: 55, and the second moiety comprising the sequence of SEQ ID NO: 9.

In some embodiments, the polypeptide comprises, from the N-terminus to the C-terminus, the first moiety comprising the sequence of SEQ ID NO: 13, the second moiety comprising the sequence of SEQ ID NO: 9 and the third moiety comprising the Fc domain having the amino acid modifications of a)T307Q, Q311V and A378V; b) F234V, L235E and D265G; and c) S228P.

In some embodiments, the polypeptide comprises, from the N-terminus to the C-terminus, the first moiety comprising the sequence of SEQ ID NO: 13, the second moiety comprising the sequence of SEQ ID NO: 9 and the third moiety comprising the Fc domain having the amino acid modification of S228P.

In some embodiments, the polypeptide comprises, from the N-terminus to the C-terminus, the first moiety comprising the sequence of SEQ ID NO: 10, the second moiety comprising the sequence of SEQ ID NO: 9 and the third moiety comprising the Fc domain having the amino acid modifications a)T307Q, Q311V and A378V; b) F234V, L235E and D265G; and c) S228P.

In some embodiments, the polypeptide comprises, from N-terminus to the C-terminus, the third moiety comprising SEQ ID NO: 31, the variable domain of the first moiety comprising a HCDR1 of SEQ ID NO: 16, a HCDR2 of SEQ ID NO: 17, and a HCDR3 of SEQ ID NO: 18, and the second moiety comprising the domains 2-4 of MCP.

In some embodiments, the polypeptide comprises, from N-terminus to the C-terminus, the third moiety comprising SEQ ID NO: 33, the variable domain of the first moiety comprising a HCDR1 of SEQ ID NO: 16, a HCDR2 of SEQ ID NO: 17, and a HCDR3 of SEQ ID NO: 18, and the second moiety comprising the domains 2-4 of MCP.

In some embodiments, the polypeptide comprises, from N-terminus to the C-terminus, the third moiety comprising SEQ ID NO: 36, the variable domain of the first moiety comprising a HCDR1 of SEQ ID NO: 16, a HCDR2 of SEQ ID NO: 17, and a HCDR3 of SEQ ID NO: 18, and the second moiety comprising the domains 2-4 of MCP.

In some embodiments, the polypeptide comprises, from N-terminus to the C-terminus, the third moiety comprising SEQ ID NO: 31, the variable domain of the first moiety comprising a HCDR1 of SEQ ID NO: 19, a HCDR2 of SEQ ID NO: 20, and a HCDR3 of SEQ ID NO: 18, and the second moiety comprising the domains 2-4 of MCP.

In some embodiments, the polypeptide comprises, from N-terminus to the C-terminus, the third moiety comprising SEQ ID NO: 33, the variable domain of the first moiety comprising a HCDR1 of SEQ ID NO: 19, a HCDR2 of SEQ ID NO: 20, and a HCDR3 of SEQ ID NO: 18, and the second moiety comprising the domains 2-4 of MCP.

In some embodiments, the polypeptide comprises, from N-terminus to the C-terminus, the third moiety comprising SEQ ID NO: 36, the variable domain of the first moiety comprising a HCDR1 of SEQ ID NO: 19, a HCDR2 of SEQ ID NO: 20, and a HCDR3 of SEQ ID NO: 18, and the second moiety comprising the domains 2-4 of MCP.

In some embodiments, the polypeptide comprises, from N-terminus to the C-terminus, the third moiety comprising SEQ ID NO: 31, the variable domain of the first moiety comprising a HCDR1 of SEQ ID NO: 16, a HCDR2 of SEQ ID NO: 26, and a HCDR3 of SEQ ID NO: 22, and the second moiety comprising the domains 2-4 of MCP.

In some embodiments, the polypeptide comprises, from N-terminus to the C-terminus, the third moiety comprising SEQ ID NO: 33, the variable domain of the first moiety comprising a HCDR1 of SEQ ID NO: 16, a HCDR2 of SEQ ID NO: 26, and a HCDR3 of SEQ ID NO: 22, and the second moiety comprising the domains 2-4 of MCP.

In some embodiments, the polypeptide comprises, from N-terminus to the C-terminus, the third moiety comprising SEQ ID NO: 36, the variable domain of the first moiety comprising a HCDR1 of SEQ ID NO: 16, a HCDR2 of SEQ ID NO: 26, and a HCDR3 of SEQ ID NO: 22, and the second moiety comprising the domains 2-4 of MCP.

In some embodiments, the polypeptide comprises, from N-terminus to the C-terminus, the third moiety comprising SEQ ID NO: 31, the variable domain of the first moiety comprising a HCDR1 of SEQ ID NO: 19, a HCDR2 of SEQ ID NO: 28, and a HCDR3 of SEQ ID NO: 27, and the second moiety comprising the domains 2-4 of MCP.

In some embodiments, the polypeptide comprises, from N-terminus to the C-terminus, the third moiety comprising SEQ ID NO: 33, the variable domain of the first moiety comprising a HCDR1 of SEQ ID NO: 19, a HCDR2 of SEQ ID NO: 28, and a HCDR3 of SEQ ID NO: 27, and the second moiety comprising the domains 2-4 of MCP.

In some embodiments, the polypeptide comprises, from N-terminus to the C-terminus, the third moiety comprising SEQ ID NO: 36, the variable domain of the first moiety comprising a HCDR1 of SEQ ID NO: 19, a HCDR2 of SEQ ID NO: 28, and a HCDR3 of SEQ ID NO: 27, and the second moiety comprising the domains 2-4 of MCP.

In some embodiments, the polypeptide comprises, from the N-terminus to the C-terminus, the third moiety comprising SEQ ID NO: 31, the first moiety comprising a variable domain having the sequence of SEQ ID NO: 10 and the second moiety comprising the amino acid sequence of SEQ ID NO: 9.

In some embodiments, the polypeptide comprises, from the N-terminus to the C-terminus, the third moiety comprising SEQ ID NO: 33, the first moiety comprising a variable domain having the sequence of SEQ ID NO: 10 and the second moiety comprising the amino acid sequence of SEQ ID NO: 9.

In some embodiments, the polypeptide comprises, from the N-terminus to the C-terminus, the third moiety comprising SEQ ID NO: 36, the first moiety comprising a variable domain having the sequence of SEQ ID NO: 10 and the second moiety comprising the amino acid sequence of SEQ ID NO: 9.

In some embodiments, the polypeptide comprises, from the N-terminus to the C-terminus, the third moiety comprising SEQ ID NO: 31, the first moiety comprising a variable domain having the sequence of SEQ ID NO: 13 and the second moiety comprising the amino acid sequence of SEQ ID NO: 9.

In some embodiments, the polypeptide comprises, from the N-terminus to the C-terminus, the third moiety comprising SEQ ID NO: 33, the first moiety comprising a variable domain having the sequence of SEQ ID NO: 13 and the second moiety comprising the amino acid sequence of SEQ ID NO: 9.

In some embodiments, the polypeptide comprises, from the N-terminus to the C-terminus, the third moiety comprising SEQ ID NO: 36, the first moiety comprising a variable domain having the sequence of SEQ ID NO: 13 and the second moiety comprising the amino acid sequence of SEQ ID NO: 9.

In some embodiments, the polypeptide comprises, from the N-terminus to the C-terminus, the third moiety comprising SEQ ID NO: 31, the first moiety comprising a variable domain having the sequence of SEQ ID NO: 10 and the second moiety comprising the amino acid sequence of SEQ ID NO: 15.

In some embodiments, the polypeptide comprises, from the N-terminus to the C-terminus, the third moiety comprising SEQ ID NO: 33, the first moiety comprising a variable domain having the sequence of SEQ ID NO: 10 and the second moiety comprising the amino acid sequence of SEQ ID NO: 15.

In some embodiments, the polypeptide comprises, from the N-terminus to the C-terminus, the third moiety comprising SEQ ID NO: 36, the first moiety comprising a variable domain having the sequence of SEQ ID NO: 10 and the second moiety comprising the amino acid sequence of SEQ ID NO: 15.

In some embodiments, the polypeptide comprises, from the N-terminus to the C-terminus, the third moiety comprising SEQ ID NO: 31, the first moiety comprising a variable domain having the sequence of SEQ ID NO: 13 and the second moiety comprising the amino acid sequence of SEQ ID NO: 15.

In some embodiments, the polypeptide comprises, from the N-terminus to the C-terminus, the third moiety comprising SEQ ID NO: 33, the first moiety comprising a variable domain having the sequence of SEQ ID NO: 13 and the second moiety comprising the amino acid sequence of SEQ ID NO: 15.

In some embodiments, the polypeptide comprises, from the N-terminus to the C-terminus, the third moiety comprising SEQ ID NO: 36, the first moiety comprising a variable domain having the sequence of SEQ ID NO: 13 and the second moiety comprising the amino acid sequence of SEQ ID NO: 15.

In some embodiments, the polypeptide comprises, from the N-terminus to the C-terminus, the third moiety comprising SEQ ID NO: 31, the first moiety comprising a variable domain having the sequence of SEQ ID NO: 55 and the second moiety comprising the amino acid sequence of SEQ ID NO: 9.

In some embodiments, the polypeptide comprises the amino acid sequence of

(SEQ ID NO: 4)
ESKYGPPCPPCPAPEVEGGPSVFLFPPKPKDTLMISRTPEVTCVVVGVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGGGGGGSEVQLLESGGGL
VQPGGSLRLSCAASGRTFSNYHMGWFRQAPGQGREFVATIIRTGETIYY
ADSVKGRFTISRDNSKNTLYLQMNSLRAEDTAVYYCAAATSGWNIKTVT
PYEYWGQGTLVTVSSGGGGSGGGGSGGGGSRETCPYIRDPLNGQAVPAN
GTYEFGYQMHFICNEGYYLIGEEILYCELKGSVAIWSGKPPICEKVLCT
PPPKIKNGKHTFSEVEVFEYLDAVTYSCDPAPGPDPFSLIGESTIYCGD
NSVWSRAAPECKVVKCRFPVVENGKQISGFGKKFYYKATVMFECDKGFY
LDGSDTIVCDSNSTWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 4. In some embodiments, the polypeptide comprises an amino acid sequence at least 80% identical to SEQ ID NO: 4. In some embodiments, the polypeptide comprises an amino acid sequence at least 85% identical to SEQ ID NO: 4. In some embodiments, the polypeptide comprises an amino acid sequence at least 88% identical to SEQ ID NO: 4. In some embodiments, the polypeptide comprises an amino acid sequence at least 90% identical to SEQ ID NO: 4. In some embodiments, the polypeptide comprises an amino acid sequence at least 91% identical to SEQ ID NO: 4. In some embodiments, the polypeptide comprises an amino acid sequence at least 92% identical to SEQ ID NO: 4. In some embodiments, the polypeptide comprises an amino acid sequence at least 93% identical to SEQ ID NO: 4. In some embodiments, the polypeptide comprises an amino acid sequence at least 94% identical to SEQ ID NO: 4. In some embodiments, the polypeptide comprises an amino acid sequence at least 95% identical to SEQ ID NO: 4. In some embodiments, the polypeptide comprises an amino acid sequence at least 96% identical to SEQ ID NO: 4. In some embodiments, the polypeptide comprises an amino acid sequence at least 97% identical to SEQ ID NO: 4. In some embodiments, the polypeptide comprises an amino acid sequence at least 98% identical to SEQ ID NO: 4. In some embodiments, the polypeptide comprises an amino acid sequence at least 99% identical to SEQ ID NO: 4.

In some embodiments, the polypeptide comprises the amino acid sequence of

(SEQ ID NO: 3)
ESKYGPPCPPCPAPEVEGGPSVFLFPPKPKDTLMISRTPEVTCVVVGVSQ
EDPEVQFNWYVDGVEVHNAKTKPREEQFNSTYRVVSVLQVLHVDWLNGKE
YKCKVSNKGLPSSIEKTISKAKGQPREPQVYTLPPSQEEMTKNQVSLTCL
VKGFYPSDIVVEWESNGQPENNYKTTPPVLDSDGSFFLYSRLTVDKSRWQ
EGNVFSCSVMHEALHNHYTQKSLSLSLGGGGGGSEVQLLESGGGLVQPGG
SLRLSCAASGRTFSNYHMGWFRQAPGQGREFVATIIRTGETIYYADSVKG
RFTISRDNSKNTLYLQMNSLRAEDTAVYYCAAATSGWNIKTVTPYEYWGQ
GTLVTVSSGGGGSGGGGSGGGGSRETCPYIRDPLNGQAVPANGTYEFGYQ
MHFICNEGYYLIGEEILYCELKGSVAIWSGKPPICEKVLCTPPPKIKNGK
HTFSEVEVFEYLDAVTYSCDPAPGPDPFSLIGESTIYCGDNSVWSRAAPE
CKVVKCRFPVVENGKQISGFGKKFYYKATVMFECDKGFYLDGSDTIVCDS
NSTWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 3. In some embodiments, the polypeptide comprises an amino acid sequence at least 80% identical to SEQ ID NO: 3. In some embodiments, the polypeptide comprises an amino acid sequence at least 85% identical to SEQ ID NO: 3. In some embodiments, the polypeptide comprises an amino acid sequence at least 88% identical to SEQ ID NO: 3. In some embodiments, the polypeptide comprises an amino acid sequence at least 90% identical to SEQ ID NO: 3. In some embodiments, the polypeptide comprises an amino acid sequence at least 91% identical to SEQ ID NO: 3. In some embodiments, the polypeptide comprises an amino acid sequence at least 92% identical to SEQ ID NO: 3. In some embodiments, the polypeptide comprises an amino acid sequence at least 93% identical to SEQ ID NO: 3. In some embodiments, the polypeptide comprises an amino acid sequence at least 94% identical to SEQ ID NO: 3. In some embodiments, the polypeptide comprises an amino acid sequence at least 95% identical to SEQ ID NO: 3. In some embodiments, the polypeptide comprises an amino acid sequence at least 96% identical to SEQ ID NO: 3. In some embodiments, the polypeptide comprises an amino acid sequence at least 97% identical to SEQ ID NO: 3. In some embodiments, the polypeptide comprises an amino acid sequence at least 98% identical to SEQ ID NO: 3. In some embodiments, the polypeptide comprises an amino acid sequence at least 99% identical to SEQ ID NO: 3.

In some embodiments, the polypeptide comprises the amino acid sequence of

(SEQ ID NO: 6)
DKTHTCPPCPAPEVEGGPSVFLFPPKPKDTLMISRTPEVTCVVVGVSHED
PEVKFNWYVDGVEVHNAKTKPREEQYNSTYRVVSVLQVLHVDWLNGKEYK
CKVSNKALPAPIEKTISKAKGQPREPQVYTLPPSREEMTKNQVSLTCLVK
GFYPSDIVVEWESNGQPENNYKTTPPVLDSDGSFFLYSKLTVDKSRWQQG
NVFSCSVMHEALHNHYTQKSLSLSPGGGGGGSEVQLLESGGGLVQPGGSL
RLSCAASGRTFSNYHMGWFRQAPGQGREFVATIIRTGETIYYADSVKGRF
TISRDNSKNTLYLQMNSLRAEDTAVYYCAAATSGWNIKTVTPYEYWGQGT
LVTVSSGGGGSGGGGSGGGGSRETCPYIRDPLNGQAVPANGTYEFGYQMH
FICNEGYYLIGEEILYCELKGSVAIWSGKPPICEKVLCTPPPKIKNGKHT
FSEVEVFEYLDAVTYSCDPAPGPDPFSLIGESTIYCGDNSVWSRAAPECK
VVKCRFPVVENGKQISGFGKKFYYKATVMFECDKGFYLDGSDTIVCDSNS
TWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 6. In some embodiments, the polypeptide comprises an amino acid sequence at least 80% identical to SEQ ID NO: 6. In some embodiments, the polypeptide comprises an amino acid sequence at least 85% identical to SEQ ID NO: 6. In some embodiments, the polypeptide comprises an amino acid sequence at least 88% identical to SEQ ID NO: 6. In some embodiments, the polypeptide comprises an amino acid sequence at least 90% identical to SEQ ID NO: 6. In some embodiments, the polypeptide comprises an amino acid sequence at least 91% identical to SEQ ID NO: 6. In some embodiments, the polypeptide comprises an amino acid sequence at least 92% identical to SEQ ID NO: 6. In some embodiments, the polypeptide comprises an amino acid sequence at least 93% identical to SEQ ID NO: 6. In some embodiments, the polypeptide comprises an amino acid sequence at least 94% identical to SEQ ID NO: 6. In some embodiments, the polypeptide comprises an amino acid sequence at least 95% identical to SEQ ID NO: 6. In some embodiments, the polypeptide comprises an amino acid sequence at least 96% identical to SEQ ID NO: 6. In some embodiments, the polypeptide comprises an amino acid sequence at least 97% identical to SEQ ID NO: 6. In some embodiments, the polypeptide comprises an amino acid sequence at least 98% identical to SEQ ID NO: 6. In some embodiments, the polypeptide comprises an amino acid sequence at least 99% identical to SEQ ID NO: 6.

In some embodiments, the polypeptide comprises the amino acid sequence of

(SEQ ID NO: 7)
ESKYGPPCPPCPAPEVEGGPSVFLFPPKPKDTLMISRTPEVTCVVVGVSQ
EDPEVQFNWYVDGVEVHNAKTKPREEQFNSTYRVVSVLQVLHVDWLNGKE
YKCKVSNKGLPSSIEKTISKAKGQPREPQVYTLPPSQEEMTKNQVSLTCL
VKGFYPSDIVVEWESNGQPENNYKTTPPVLDSDGSFFLYSRLTVDKSRWQ
EGNVFSCSVMHEALHNHYTQKSLSLSLGGGGGGSEVQLLESGGGLVQPGG
SLRLSCAASGRTFSNYHMGWFRQAPGQGREFVATIIRTGETIYYADSVKG
RFTISRDNSKNTLYLQMNSLRAEDTAVYYCAAATSGWNIKTVTPYEYWGQ
GTLVTVSSGGGGSGGGGSGGGGSRETCPYIRDPLNGQAVPANGTYEFGYQ
MHFICNEGYYLIGEEILYCELKGSVAIWSGKPPICEKVLCTPPPKIKNGK
HTFSEVEVFEYLDAVTYSCDPAPGPDPFSLIGESTIYCGDNSVWSRAAPE
CKVVKCRFPVVENGKQISGFGKKFYYKATVMFECDKGFYLDGSDTIVCDR
NSTWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 7. In some embodiments, the polypeptide comprises an amino acid sequence at least 80% identical to SEQ ID NO: 7. In some embodiments, the polypeptide comprises an amino acid sequence at least 85% identical to SEQ ID NO: 7. In some embodiments, the polypeptide comprises an amino acid sequence at least 88% identical to SEQ ID NO: 7. In some embodiments, the polypeptide comprises an amino acid sequence at least 90% identical to SEQ ID NO: 7. In some embodiments, the polypeptide comprises an amino acid sequence at least 91% identical to SEQ ID NO: 7. In some embodiments, the polypeptide comprises an amino acid sequence at least 92% identical to SEQ ID NO: 7. In some embodiments, the polypeptide comprises an amino acid sequence at least 93% identical to SEQ ID NO: 7. In some embodiments, the polypeptide comprises an amino acid sequence at least 94% identical to SEQ ID NO: 7. In some embodiments, the polypeptide comprises an amino acid sequence at least 95% identical to SEQ ID NO: 7. In some embodiments, the polypeptide comprises an amino acid sequence at least 96% identical to SEQ ID NO: 7. In some embodiments, the polypeptide comprises an amino acid sequence at least 97% identical to SEQ ID NO: 7. In some embodiments, the polypeptide comprises an amino acid sequence at least 98% identical to SEQ ID NO: 7. In some embodiments, the polypeptide comprises an amino acid sequence at least 99% identical to SEQ ID NO: 7.

In some embodiments, the polypeptide comprises the amino acid sequence of

(SEQ ID NO: 43)
EVQLLESGGGLVQPGGSLRLSCAASGRTFSNYHMGWFRQAPGQGREFVAT
IIRHGTTIYYADSVKGRFTISRDNSKNTLYLQMNSLRAEDTAVYYCAAAT
SGWHIKTVTPYEYWGQGTLVTVSSGGGGSGGGGSGGGGSRETCPYIRDPL
NGQAVPANGTYEFGYQMHFICNEGYYLIGEEILYCELKGSVAIWSGKPPI
CEKVLCTPPPKIKNGKHTFSEVEVFEYLDAVTYSCDPAPGPDPFSLIGES
TIYCGDNSVWSRAAPECKVVKCRFPVVENGKQISGFGKKFYYKATVMFEC
DKGFYLDGSDTIVCDSNSTWDPPVPKCLKVESKYGPPCPPCPAPEVEGGP
SVFLFPPKPKDTLMISRTPEVTCVVVGVSQEDPEVQFNWYVDGVEVHNAK
TKPREEQFNSTYRVVSVLQVLHVDWLNGKEYKCKVSNKGLPSSIEKTISK
AKGQPREPQVYTLPPSQEEMTKNQVSLTCLVKGFYPSDIVVEWESNGQPE
NNYKTTPPVLDSDGSFFLYSRLTVDKSRWQEGNVFSCSVMHEALHNHYTQ
KSLSLSLGK.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 43. In some embodiments, the polypeptide comprises an amino acid sequence at least 80% identical to SEQ ID NO: 43. In some embodiments, the polypeptide comprises an amino acid sequence at least 85% identical to SEQ ID NO: 43. In some embodiments, the polypeptide comprises an amino acid sequence at least 88% identical to SEQ ID NO: 43. In some embodiments, the polypeptide comprises an amino acid sequence at least 90% identical to SEQ ID NO: 43. In some embodiments, the polypeptide comprises an amino acid sequence at least 91% identical to SEQ ID NO: 43. In some embodiments, the polypeptide comprises an amino acid sequence at least 92% identical to SEQ ID NO: 43. In some embodiments, the polypeptide comprises an amino acid sequence at least 93% identical to SEQ ID NO: 43. In some embodiments, the polypeptide comprises an amino acid sequence at least 94% identical to SEQ ID NO: 43. In some embodiments, the polypeptide comprises an amino acid sequence at least 95% identical to SEQ ID NO: 43. In some embodiments, the polypeptide comprises an amino acid sequence at least 96% identical to SEQ ID NO: 43. In some embodiments, the polypeptide comprises an amino acid sequence at least 97% identical to SEQ ID NO: 43. In some embodiments, the polypeptide comprises an amino acid sequence at least 98% identical to SEQ ID NO: 43. In some embodiments, the polypeptide comprises an amino acid sequence at least 99% identical to SEQ ID NO: 43.

In some embodiments, the polypeptide comprises the amino acid sequence of

(SEQ ID NO: 44)
EVQLLESGGGLVQPGGSLRLSCAASGRTFSNYHMGWFRQAPGQGREFVAT
IIRHGTTIYYADSVKGRFTISRDNSKNTLYLQMNSLRAEDTAVYYCAAAT
SGWHIKTVTPYEYWGQGTLVTVSSGGGGSGGGGSGGGGSRETCPYIRDPL
NGQAVPANGTYEFGYQMHFICNEGYYLIGEEILYCELKGSVAIWSGKPPI
CEKVLCTPPPKIKNGKHTFSEVEVFEYLDAVTYSCDPAPGPDPFSLIGES
TIYCGDNSVWSRAAPECKVVKCRFPVVENGKQISGFGKKFYYKATVMFEC
DKGFYLDGSDTIVCDSNSTWDPPVPKCLKVESKYGPPCPPCPAPEFVGGP
SVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYTQ
KSLSLSLGK.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 44. In some embodiments, the polypeptide comprises an amino acid sequence at least 80% identical to SEQ ID NO: 44. In some embodiments, the polypeptide comprises an amino acid sequence at least 85% identical to SEQ ID NO: 44. In some embodiments, the polypeptide comprises an amino acid sequence at least 88% identical to SEQ ID NO: 44. In some embodiments, the polypeptide comprises an amino acid sequence at least 90% identical to SEQ ID NO: 44. In some embodiments, the polypeptide comprises an amino acid sequence at least 91% identical to SEQ ID NO: 44. In some embodiments, the polypeptide comprises an amino acid sequence at least 92% identical to SEQ ID NO: 44. In some embodiments, the polypeptide comprises an amino acid sequence at least 93% identical to SEQ ID NO: 44. In some embodiments, the polypeptide comprises an amino acid sequence at least 94% identical to SEQ ID NO: 44. In some embodiments, the polypeptide comprises an amino acid sequence at least 95% identical to SEQ ID NO: 44. In some embodiments, the polypeptide comprises an amino acid sequence at least 96% identical to SEQ ID NO: 44. In some embodiments, the polypeptide comprises an amino acid sequence at least 97% identical to SEQ ID NO: 44. In some embodiments, the polypeptide comprises an amino acid sequence at least 98% identical to SEQ ID NO: 44. In some embodiments, the polypeptide comprises an amino acid sequence at least 99% identical to SEQ ID NO: 44.

In some embodiments, the polypeptide comprises the amino acid sequence of

(SEQ ID NO: 45)
EVQLLESGGGLVQPGGSLRLSCAASGRTFSNYHMGWFRQAPGQGREFVAT
IIRHGTTIYYADSVKGRFTISRDNSKNTLYLQMNSLRAEDTAVYYCAAAT
SGWHIKTVTPYEYWGQGTLVTVSSGGGGSGGGGSRETCPYIRDPLNGQAV
PANGTYEFGYQMHFICNEGYYLIGEEILYCELKGSVAIWSGKPPICEKVL
CTPPPKIKNGKHTFSEVEVFEYLDAVTYSCDPAPGPDPFSLIGESTIYCG
DNSVWSRAAPECKVVKCRFPVVENGKQISGFGKKFYYKATVMFECDKGFY
LDGSDTIVCDSNSTWDPPVPKCLKVESKYGPPCPPCPAPEVEGGPSVFLF
PPKPKDTLMISRTPEVTCVVVVSQEDPEVQFNWYVDGVEVHNAKTKPREE
QFNSTYRVVSVLQVLHVDWLNGKEYKCKVSNKGLPSSIEKTISKAKGQPR
EPQVYTLPPSQEEMTKNQVSLTCLVKGFYPSDIVVEWESNGQPENNYKTT
PPVLDSDGSFFLYSRLTVDKSRWQEGNVFSCSVMHEALHNHYTQKSLSLS
LGK.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 45. In some embodiments, the polypeptide comprises an amino acid sequence at least 80% identical to SEQ ID NO: 45. In some embodiments, the polypeptide comprises an amino acid sequence at least 85% identical to SEQ ID NO: 45. In some embodiments, the polypeptide comprises an amino acid sequence at least 88% identical to SEQ ID NO: 45. In some embodiments, the polypeptide comprises an amino acid sequence at least 90% identical to SEQ ID NO: 45. In some embodiments, the polypeptide comprises an amino acid sequence at least 91% identical to SEQ ID NO: 45. In some embodiments, the polypeptide comprises an amino acid sequence at least 92% identical to SEQ ID NO: 45. In some embodiments, the polypeptide comprises an amino acid sequence at least 93% identical to SEQ ID NO: 45. In some embodiments, the polypeptide comprises an amino acid sequence at least 94% identical to SEQ ID NO: 45. In some embodiments, the polypeptide comprises an amino acid sequence at least 95% identical to SEQ ID NO: 45. In some embodiments, the polypeptide comprises an amino acid sequence at least 96% identical to SEQ ID NO: 45. In some embodiments, the polypeptide comprises an amino acid sequence at least 97% identical to SEQ ID NO: 45. In some embodiments, the polypeptide comprises an amino acid sequence at least 98% identical to SEQ ID NO: 45. In some embodiments, the polypeptide comprises an amino acid sequence at least 99% identical to SEQ ID NO: 45. In some embodiments, the polypeptide comprises the amino acid sequence of

(SEQ ID NO: 46)
EVQLLESGGGLVQPGGSLRLSCAASGRTFSNYHMGWFRQAPGQGREFVAT
IIRHGTTIYYADSVKGRFTISRDNSKNTLYLQMNSLRAEDTAVYYCAAAT
SGWHIKTVTPYEYWGQGTLVTVSSGGGGSRETCPYIRDPLNGQAVPANGT
YEFGYQMHFICNEGYYLIGEEILYCELKGSVAIWSGKPPICEKVLCTPPP
KIKNGKHTFSEVEVFEYLDAVTYSCDPAPGPDPFSLIGESTIYCGDNSVW
SRAAPECKVVKCRFPVVENGKQISGFGKKFYYKATVMFECDKGFYLDGSD
TIVCDSNSTWDPPVPKCLKVESKYGPPCPPCPAPEVEGGPSVFLFPPKPK
DTLMISRTPEVTCVVVVSQEDPEVQFNWYVDGVEVHNAKTKPREEQFNST
YRVVSVLQVLHVDWLNGKEYKCKVSNKGLPSSIEKTISKAKGQPREPQVY
TLPPSQEEMTKNQVSLTCLVKGFYPSDIVVEWESNGQPENNYKTTPPVLD
SDGSFFLYSRLTVDKSRWQEGNVFSCSVMHEALHNHYTQKSLSLSLGK.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 46. In some embodiments, the polypeptide comprises an amino acid sequence at least 80% identical to SEQ ID NO: 46. In some embodiments, the polypeptide comprises an amino acid sequence at least 85% identical to SEQ ID NO: 46. In some embodiments, the polypeptide comprises an amino acid sequence at least 88% identical to SEQ ID NO: 46. In some embodiments, the polypeptide comprises an amino acid sequence at least 90% identical to SEQ ID NO: 46. In some embodiments, the polypeptide comprises an amino acid sequence at least 91% identical to SEQ ID NO: 46. In some embodiments, the polypeptide comprises an amino acid sequence at least 92% identical to SEQ ID NO: 46. In some embodiments, the polypeptide comprises an amino acid sequence at least 93% identical to SEQ ID NO: 46. In some embodiments, the polypeptide comprises an amino acid sequence at least 94% identical to SEQ ID NO: 46. In some embodiments, the polypeptide comprises an amino acid sequence at least 95% identical to SEQ ID NO: 46. In some embodiments, the polypeptide comprises an amino acid sequence at least 96% identical to SEQ ID NO: 46. In some embodiments, the polypeptide comprises an amino acid sequence at least 97% identical to SEQ ID NO: 46. In some embodiments, the polypeptide comprises an amino acid sequence at least 98% identical to SEQ ID NO: 46. In some embodiments, the polypeptide comprises an amino acid sequence at least 99% identical to SEQ ID NO: 46.

In some embodiments, the polypeptide comprises the amino acid sequence of

(SEQ ID NO: 47)
ESKYGPPCPPCPAPEVEGGPSVFLFPPKPKDTLMISRTPEVTCVVVGVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGGGGGGSEVQLLESGGGLVQPGG
SLRLSCAASGRTFSNYHMGWFRQAPGQGREFVATIIRHGTTIYYADSVKG
RFTISRDNSKNTLYLQMNSLRAEDTAVYYCAAATSGWHIKTVTPYEYWGQ
GTLVTVSSGGGGSGGGGSGGGGSRETCPYIRDPLNGQAVPANGTYEFGYQ
MHFICNEGYYLIGEEILYCELKGSVAIWSGKPPICEKVLCTPPPKIKNGK
HTFSEVEVFEYLDAVTYSCDPAPGPDPFSLIGESTIYCGDNSVWSRAAPE
CKVVKCRFPVVENGKQISGFGKKFYYKATVMFECDKGFYLDGSDTIVCDS
NSTWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 47. In some embodiments, the polypeptide comprises an amino acid sequence at least 80% identical to SEQ ID NO: 47. In some embodiments, the polypeptide comprises an amino acid sequence at least 85% identical to SEQ ID NO: 47. In some embodiments, the polypeptide comprises an amino acid sequence at least 88% identical to SEQ ID NO: 47. In some embodiments, the polypeptide comprises an amino acid sequence at least 90% identical to SEQ ID NO: 47. In some embodiments, the polypeptide comprises an amino acid sequence at least 91% identical to SEQ ID NO: 47. In some embodiments, the polypeptide comprises an amino acid sequence at least 92% identical to SEQ ID NO: 47. In some embodiments, the polypeptide comprises an amino acid sequence at least 93% identical to SEQ ID NO: 47. In some embodiments, the polypeptide comprises an amino acid sequence at least 94% identical to SEQ ID NO: 47. In some embodiments, the polypeptide comprises an amino acid sequence at least 95% identical to SEQ ID NO: 47. In some embodiments, the polypeptide comprises an amino acid sequence at least 96% identical to SEQ ID NO: 47. In some embodiments, the polypeptide comprises an amino acid sequence at least 97% identical to SEQ ID NO: 47. In some embodiments, the polypeptide comprises an amino acid sequence at least 98% identical to SEQ ID NO: 47. In some embodiments, the polypeptide comprises an amino acid sequence at least 99% identical to SEQ ID NO: 47.

In some embodiments, the polypeptide comprises the amino acid sequence of

(SEQ ID NO: 48)
ESKYGPPCPPCPAPEVEGGPSVFLFPPKPKDTLMISRTPEVTCVVVGVSQ
EDPEVQFNWYVDGVEVHNAKTKPREEQFNSTYRVVSVLQVLHVDWLNGKE
YKCKVSNKGLPSSIEKTISKAKGQPREPQVYTLPPSQEEMTKNQVSLTCL
VKGFYPSDIVVEWESNGQPENNYKTTPPVLDSDGSFFLYSRLTVDKSRWQ
EGNVFSCSVMHEALHNHYTQKSLSLSLGGGGGGSEVQLLESGGGLVQPGG
SLRLSCAASGRTFSNYHMGWFRQAPGQGREFVATIIRHGTTIYYADSVKG
RFTISRDNSKNTLYLQMNSLRAEDTAVYYCAAATSGWHIKTVTPYEYWGQ
GTLVTVSSGGGGSGGGGSGGGGSRETCPYIRDPLNGQAVPANGTYEFGYQ
MHFICNEGYYLIGEEILYCELKGSVAIWSGKPPICEKVLCTPPPKIKNGK
HTFSEVEVFEYLDAVTYSCDPAPGPDPFSLIGESTIYCGDNSVWSRAAPE
CKVVKCRFPVVENGKQISGFGKKFYYKATVMFECDKGFYLDGSDTIVCDS
NSTWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 48. In some embodiments, the polypeptide comprises an amino acid sequence at least 80% identical to SEQ ID NO: 48. In some embodiments, the polypeptide comprises an amino acid sequence at least 85% identical to SEQ ID NO: 48. In some embodiments, the polypeptide comprises an amino acid sequence at least 88% identical to SEQ ID NO: 48. In some embodiments, the polypeptide comprises an amino acid sequence at least 90% identical to SEQ ID NO: 48. In some embodiments, the polypeptide comprises an amino acid sequence at least 91% identical to SEQ ID NO: 48. In some embodiments, the polypeptide comprises an amino acid sequence at least 92% identical to SEQ ID NO: 48. In some embodiments, the polypeptide comprises an amino acid sequence at least 93% identical to SEQ ID NO: 48. In some embodiments, the polypeptide comprises an amino acid sequence at least 94% identical to SEQ ID NO: 48. In some embodiments, the polypeptide comprises an amino acid sequence at least 95% identical to SEQ ID NO: 48. In some embodiments, the polypeptide comprises an amino acid sequence at least 96% identical to SEQ ID NO: 48. In some embodiments, the polypeptide comprises an amino acid sequence at least 97% identical to SEQ ID NO: 48. In some embodiments, the polypeptide comprises an amino acid sequence at least 98% identical to SEQ ID NO: 48. In some embodiments, the polypeptide comprises an amino acid sequence at least 99% identical to SEQ ID NO: 48.

In some embodiments, the polypeptide comprises the amino acid sequence of

(SEQ ID NO: 49)
ESKYGPPCPPCPAPEFVGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGGGGGGSEVQLLESGGGLVQPGG
SLRLSCAASGRTFSNYHMGWFRQAPGQGREFVATIIRHGTTIYYADSVKG
RFTISRDNSKNTLYLQMNSLRAEDTAVYYCAAATSGWHIKTVTPYEYWGQ
GTLVTVSSGGGGSGGGGSGGGGSRETCPYIRDPLNGQAVPANGTYEFGYQ
MHFICNEGYYLIGEEILYCELKGSVAIWSGKPPICEKVLCTPPPKIKNGK
HTFSEVEVFEYLDAVTYSCDPAPGPDPFSLIGESTIYCGDNSVWSRAAPE
CKVVKCRFPVVENGKQISGFGKKFYYKATVMFECDKGFYLDGSDTIVCDS
NSTWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 49. In some embodiments, the polypeptide comprises an amino acid sequence at least 80% identical to SEQ ID NO: 49. In some embodiments, the polypeptide comprises an amino acid sequence at least 85% identical to SEQ ID NO: 49. In some embodiments, the polypeptide comprises an amino acid sequence at least 88% identical to SEQ ID NO: 49. In some embodiments, the polypeptide comprises an amino acid sequence at least 90% identical to SEQ ID NO: 49. In some embodiments, the polypeptide comprises an amino acid sequence at least 91% identical to SEQ ID NO: 49. In some embodiments, the polypeptide comprises an amino acid sequence at least 92% identical to SEQ ID NO: 49. In some embodiments, the polypeptide comprises an amino acid sequence at least 93% identical to SEQ ID NO: 49. In some embodiments, the polypeptide comprises an amino acid sequence at least 94% identical to SEQ ID NO: 49. In some embodiments, the polypeptide comprises an amino acid sequence at least 95% identical to SEQ ID NO: 49. In some embodiments, the polypeptide comprises an amino acid sequence at least 96% identical to SEQ ID NO: 49. In some embodiments, the polypeptide comprises an amino acid sequence at least 97% identical to SEQ ID NO: 49. In some embodiments, the polypeptide comprises an amino acid sequence at least 98% identical to SEQ ID NO: 49. In some embodiments, the polypeptide comprises an amino acid sequence at least 99% identical to SEQ ID NO: 49.

In some embodiments, the polypeptide comprises the amino acid sequence of

(SEQ ID NO: 50)
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSPGGGGGSEVQLLESGGGLVQPGGS
LRLSCAASGRTFSNYHMGWFRQAPGQGREFVATIIRTGETIYYADSVKGR
FTISRDNSKNTLYLQMNSLRAEDTAVYYCAAATSGWNIKTVTPYEYWGQG
TLVTVSSGGGGSGGGGSGGGGSRETCPYIRDPLNGQAVPANGTYEFGYQM
HFICNEGYYLIGEEILYCELKGSVAIWSGKPPICEKVLCTPPPKIKNGKH
TFSEVEVFEYLDAVTYSCDPAPGPDPFSLIGESTIYCGDNSVWSRAAPEC
KVVKCRFPVVENGKQISGFGKKFYYKATVMFECDKGFYLDGSDTIVCDSN
STWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 50. In some embodiments, the polypeptide comprises an amino acid sequence at least 80% identical to SEQ ID NO: 50. In some embodiments, the polypeptide comprises an amino acid sequence at least 85% identical to SEQ ID NO: 50. In some embodiments, the polypeptide comprises an amino acid sequence at least 88% identical to SEQ ID NO: 50. In some embodiments, the polypeptide comprises an amino acid sequence at least 90% identical to SEQ ID NO: 50. In some embodiments, the polypeptide comprises an amino acid sequence at least 91% identical to SEQ ID NO: 50. In some embodiments, the polypeptide comprises an amino acid sequence at least 92% identical to SEQ ID NO: 50. In some embodiments, the polypeptide comprises an amino acid sequence at least 93% identical to SEQ ID NO: 50. In some embodiments, the polypeptide comprises an amino acid sequence at least 94% identical to SEQ ID NO: 50. In some embodiments, the polypeptide comprises an amino acid sequence at least 95% identical to SEQ ID NO: 50. In some embodiments, the polypeptide comprises an amino acid sequence at least 96% identical to SEQ ID NO: 50. In some embodiments, the polypeptide comprises an amino acid sequence at least 97% identical to SEQ ID NO: 50. In some embodiments, the polypeptide comprises an amino acid sequence at least 98% identical to SEQ ID NO: 50. In some embodiments, the polypeptide comprises an amino acid sequence at least 99% identical to SEQ ID NO: 50.

In some embodiments, the polypeptide comprises the amino acid sequence of

(SEQ ID NO: 51)
EVQLLESGGGLVQPGGSLRLSCAASGRTFSNYHMGWFRQAPGQGREFVAT
IIRTGETIYYADSVKGRFTISRDNSKNTLYLQMNSLRAEDTAVYYCAAAT
SGWNIKTVTPYEYWGQGTLVTVSSGGGGSGGGGSGGGGSRETCPYIRDPL
NGQAVPANGTYEFGYQMHFICNEGYYLIGEEILYCELKGSVAIWSGKPPI
CEKVLCTPPPKIKNGKHTFSEVEVFEYLDAVTYSCDPAPGPDPFSLIGES
TIYCGDNSVWSRAAPECKVVKCRFPVVENGKQISGFGKKFYYKATVMFEC
DKGFYLDGSDTIVCDSNSTWDPPVPKCLKVESKYGPPCPPCPAPEVEGGP
SVFLFPPKPKDTLMISRTPEVTCVVVVSQEDPEVQFNWYVDGVEVHNAKT
KPREEQFNSTYRVVSVLQVLHVDWLNGKEYKCKVSNKGLPSSIEKTISKA
KGQPREPQVYTLPPSQEEMTKNQVSLTCLVKGFYPSDIVVEWESNGQPEN
NYKTTPPVLDSDGSFFLYSRLTVDKSRWQEGNVFSCSVMHEALHNHYTQK
SLSLSLGK.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 51. In some embodiments, the polypeptide comprises an amino acid sequence at least 80% identical to SEQ ID NO: 51. In some embodiments, the polypeptide comprises an amino acid sequence at least 85% identical to SEQ ID NO: 51. In some embodiments, the polypeptide comprises an amino acid sequence at least 88% identical to SEQ ID NO: 51. In some embodiments, the polypeptide comprises an amino acid sequence at least 90% identical to SEQ ID NO: 51. In some embodiments, the polypeptide comprises an amino acid sequence at least 91% identical to SEQ ID NO: 51. In some embodiments, the polypeptide comprises an amino acid sequence at least 92% identical to SEQ ID NO: 51. In some embodiments, the polypeptide comprises an amino acid sequence at least 93% identical to SEQ ID NO: 51. In some embodiments, the polypeptide comprises an amino acid sequence at least 94% identical to SEQ ID NO: 51. In some embodiments, the polypeptide comprises an amino acid sequence at least 95% identical to SEQ ID NO: 51. In some embodiments, the polypeptide comprises an amino acid sequence at least 96% identical to SEQ ID NO: 51. In some embodiments, the polypeptide comprises an amino acid sequence at least 97% identical to SEQ ID NO: 51. In some embodiments, the polypeptide comprises an amino acid sequence at least 98% identical to SEQ ID NO: 51. In some embodiments, the polypeptide comprises an amino acid sequence at least 99% identical to SEQ ID NO: 51.

In some embodiments, the polypeptide comprises the amino acid sequence of

(SEQ ID NO: 52)
EVQLLESGGGLVQPGGSLRLSCAASGRTFSNYHMGWFRQAPGQGREFVAT
IIRHGTTIYYADSVKGRFTISRDNSKNTLYLQMNSLRAEDTAVYYCAAAT
SGWHIKTVTPYEYWGQGTLVTVSSESKYGPPCPPCPAPEVEGGPSVFLFP
PKPKDTLMISRTPEVTCVVVGVSQEDPEVQFNWYVDGVEVHNAKTKPREE
QFNSTYRVVSVLQVLHVDWLNGKEYKCKVSNKGLPSSIEKTISKAKGQPR
EPQVYTLPPSQEEMTKNQVSLTCLVKGFYPSDIVVEWESNGQPENNYKTT
PPVLDSDGSFFLYSRLTVDKSRWQEGNVFSCSVMHEALHNHYTQKSLSLS
LGKGGGGSGGGGSRETCPYIRDPLNGQAVPANGTYEFGYQMHFICNEGYY
LIGEEILYCELKGSVAIWSGKPPICEKVLCTPPPKIKNGKHTFSEVEVFE
YLDAVTYSCDPAPGPDPFSLIGESTIYCGDNSVWSRAAPECKVVKCRFPV
VENGKQISGFGKKFYYKATVMFECDKGFYLDGSDTIVCDSNSTWDPPVPK
CLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 52. In some embodiments, the polypeptide comprises an amino acid sequence at least 80% identical to SEQ ID NO: 52. In some embodiments, the polypeptide comprises an amino acid sequence at least 85% identical to SEQ ID NO: 52. In some embodiments, the polypeptide comprises an amino acid sequence at least 88% identical to SEQ ID NO: 52. In some embodiments, the polypeptide comprises an amino acid sequence at least 90% identical to SEQ ID NO: 52. In some embodiments, the polypeptide comprises an amino acid sequence at least 91% identical to SEQ ID NO: 52. In some embodiments, the polypeptide comprises an amino acid sequence at least 92% identical to SEQ ID NO: 52. In some embodiments, the polypeptide comprises an amino acid sequence at least 93% identical to SEQ ID NO: 52. In some embodiments, the polypeptide comprises an amino acid sequence at least 94% identical to SEQ ID NO: 52. In some embodiments, the polypeptide comprises an amino acid sequence at least 95% identical to SEQ ID NO: 52. In some embodiments, the polypeptide comprises an amino acid sequence at least 96% identical to SEQ ID NO: 52. In some embodiments, the polypeptide comprises an amino acid sequence at least 97% identical to SEQ ID NO: 52. In some embodiments, the polypeptide comprises an amino acid sequence at least 98% identical to SEQ ID NO: 52. In some embodiments, the polypeptide comprises an amino acid sequence at least 99% identical to SEQ ID NO: 52.

In some embodiments, the polypeptide comprises the amino acid sequence of

(SEQ ID NO: 53)
ESKYGPPCPPCPAPEVEGGPSVFLFPPKPKDTLMISRTPEVTCVVVGVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGGGGGGSEVQLQESGGGLVQPGG
SLRLSCAASGRTFSNYHMGWFRQAPGKEREFVGTIIRTGETIYYADSVKG
RFTISRDNDKNTVYLQMNSLKPGDTAVYYCAAATSGWNIKTVTPYEYWGQ
GTQVTVSSGGGGSGGGGSGGGGSRETCPYIRDPLNGQAVPANGTYEFGYQ
MHFICNEGYYLIGEEILYCELKGSVAIWSGKPPICEKVLCTPPPKIKNGK
HTFSEVEVFEYLDAVTYSCDPAPGPDPFSLIGESTIYCGDNSVWSRAAPE
CKVVKCRFPVVENGKQISGFGKKFYYKATVMFECDKGFYLDGSDTIVCDS
NSTWDPPVPKCLKV.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 53. In some embodiments, the polypeptide comprises an amino acid sequence at least 80% identical to SEQ ID NO: 53. In some embodiments, the polypeptide comprises an amino acid sequence at least 85% identical to SEQ ID NO: 53. In some embodiments, the polypeptide comprises an amino acid sequence at least 88% identical to SEQ ID NO: 53. In some embodiments, the polypeptide comprises an amino acid sequence at least 90% identical to SEQ ID NO: 53. In some embodiments, the polypeptide comprises an amino acid sequence at least 91% identical to SEQ ID NO: 53. In some embodiments, the polypeptide comprises an amino acid sequence at least 92% identical to SEQ ID NO: 53. In some embodiments, the polypeptide comprises an amino acid sequence at least 93% identical to SEQ ID NO: 53. In some embodiments, the polypeptide comprises an amino acid sequence at least 94% identical to SEQ ID NO: 53. In some embodiments, the polypeptide comprises an amino acid sequence at least 95% identical to SEQ ID NO: 53. In some embodiments, the polypeptide comprises an amino acid sequence at least 96% identical to SEQ ID NO: 53. In some embodiments, the polypeptide comprises an amino acid sequence at least 97% identical to SEQ ID NO: 53. In some embodiments, the polypeptide comprises an amino acid sequence at least 98% identical to SEQ ID NO: 53. In some embodiments, the polypeptide comprises an amino acid sequence at least 99% identical to SEQ ID NO: 53.

In some embodiments, the polypeptide comprises the amino acid sequence of

(SEQ ID NO: 54)
ESKYGPPCPPCPAPEVEGGPSVFLFPPKPKDTLMISRTPEVTCVVVGVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGGGGGGSEVQLLESGGGLVQPGG
SLRLSCAASGRTFSNYHMGWFRQAPGQGREFVATIIRHGTTIYYADSVKG
RFTISRDNSKNTLYLQMNSLRAEDTAVYYCAAATSGWHIKTVTPYEYWGQ
GTLVTVSSGGGGSGGGGSGGGGSRRKACSNLPDPLNGQVSYPNGDMLFGS
KAQFTCNTGFYIIGAETVYCQVSGNVMAWSEPSPLCEKILCKPPGEIPNG
KYTNSHKDVFEYNEVVTYSCLSSTGPDEFSLVGESSLFCIGKDEWSSDPP
ECKVVKCPYPVVPNGEIVSGFGSKFYYKAEVVFKCNAGFTLHGRDTIVCG
ANSTWEPEMPQCIKD.

In some embodiments, the polypeptide comprises an amino acid sequence at least 70%, 75%, 80%, 85%, 90% 91%, 92%, 93%, 94%, 95%, 96%, 97%, 98%, or 99% identical to the sequence of SEQ ID NO: 54. In some embodiments, the polypeptide comprises an amino acid sequence at least 80% identical to SEQ ID NO: 54. In some embodiments, the polypeptide comprises an amino acid sequence at least 85% identical to SEQ ID NO: 54. In some embodiments, the polypeptide comprises an amino acid sequence at least 88% identical to SEQ ID NO: 54. In some embodiments, the polypeptide comprises an amino acid sequence at least 90% identical to SEQ ID NO: 54. In some embodiments, the polypeptide comprises an amino acid sequence at least 91% identical to SEQ ID NO: 54. In some embodiments, the polypeptide comprises an amino acid sequence at least 92% identical to SEQ ID NO: 54. In some embodiments, the polypeptide comprises an amino acid sequence at least 93% identical to SEQ ID NO: 54. In some embodiments, the polypeptide comprises an amino acid sequence at least 94% identical to SEQ ID NO: 54. In some embodiments, the polypeptide comprises an amino acid sequence at least 95% identical to SEQ ID NO: 54. In some embodiments, the polypeptide comprises an amino acid sequence at least 96% identical to SEQ ID NO: 54. In some embodiments, the polypeptide comprises an amino acid sequence at least 97% identical to SEQ ID NO: 54. In some embodiments, the polypeptide comprises an amino acid sequence at least 98% identical to SEQ ID NO: 54. In some embodiments, the polypeptide comprises an amino acid sequence at least 99% identical to SEQ ID NO: 54.

In some embodiments, the present invention provides a protein that comprises two polypeptides capable of binding to C3b described herein. In some embodiments, the protein comprises two identical polypeptides (i.e., a homodimer).

In some embodiments, the protein comprises two polypeptides, each of which comprises the sequence of SEQ ID NO: 4.

In some embodiments, the protein comprises two polypeptides, each of which comprises the sequence of SEQ ID NO: 3.

In some embodiments, the protein comprises two polypeptides, each of which comprises the sequence of SEQ ID NO: 6.

In some embodiments, the protein comprises two polypeptides, each of which comprises the sequence of SEQ ID NO: 7.

In some embodiments, the protein comprises two polypeptides, each of which comprises the sequence of SEQ ID NO: 43.

In some embodiments, the protein comprises two polypeptides, each of which comprises the sequence of SEQ ID NO: 44.

In some embodiments, the protein comprises two polypeptides, each of which comprises the sequence of SEQ ID NO: 45.

In some embodiments, the protein comprises two polypeptides, each of which comprises the sequence of SEQ ID NO: 46.

In some embodiments, the protein comprises two polypeptides, each of which comprises the sequence of SEQ ID NO: 47.

In some embodiments, the protein comprises two polypeptides, each of which comprises the sequence of SEQ ID NO: 48.

In some embodiments, the protein comprises two polypeptides, each of which comprises the sequence of SEQ ID NO: 49.

In some embodiments, the protein comprises two polypeptides, each of which comprises the sequence of SEQ ID NO: 50.

In some embodiments, the protein comprises two polypeptides, each of which comprises the sequence of SEQ ID NO: 51.

In some embodiments, the protein comprises two polypeptides, each of which comprises the sequence of SEQ ID NO: 52.

In some embodiments, the protein comprises two polypeptides, each of which comprises the sequence of SEQ ID NO: 53.

In some embodiments, the protein comprises two polypeptides, each of which comprises the sequence of SEQ ID NO: 54.

In some embodiments, a protein comprises two polypeptides, wherein each polypeptide comprises from the N-terminus to the C-terminus, the first moiety comprising a variable domain capable of binding to complement C3b a linker and a second moiety comprising an amino acid sequence of a membrane complement protein (MCP), or a variant thereof.

In some embodiments, a protein comprises two polypeptides, wherein each polypeptide comprises, from the N-terminus to the C-terminus, the first moiety comprising an anti-C3bVHH and the second moiety comprising the domains 2-4 of MCP.

In some embodiments, a protein comprises two polypeptides, wherein each polypeptide comprises, from N-terminus to the C-terminus, the variable domain of the first moiety comprising a HCDR1 of SEQ ID NO: 16, a HCDR2 of SEQ ID NO: 17, and a HCDR3 of SEQ ID NO: 18, and the second moiety comprising the domains 2-4 of MCP.

In some embodiments, a protein comprises two polypeptides, wherein each polypeptide comprises, from N-terminus to the C-terminus, the variable domain of the first moiety comprising a HCDR1 of SEQ ID NO: 19, a HCDR2 of SEQ ID NO: 20, and a HCDR3 of SEQ ID NO: 18, and the second moiety comprising the domains 2-4 of MCP.

In some embodiments, a protein comprises two polypeptides, wherein each polypeptide comprises, from N-terminus to the C-terminus, the variable domain of the first moiety comprising a HCDR1 of SEQ ID NO: 16, a HCDR2 of SEQ ID NO: 26, and a HCDR3 of SEQ ID NO: 22, and the second moiety comprising the domains 2-4 of MCP.

In some embodiments, a protein comprises two polypeptides, wherein each polypeptide comprises, from N-terminus to the C-terminus, the variable domain of the first moiety comprising a HCDR1 of SEQ ID NO: 19, a HCDR2 of SEQ ID NO: 28, and a HCDR3 of SEQ ID NO: 27, and the second moiety comprising the domains 2-4 of MCP.

In some embodiments, a protein comprises two polypeptides, wherein each polypeptide comprises, from the N-terminus to the C-terminus, the first moiety comprising a variable domain having the sequence of SEQ ID NO: 10 and the second moiety comprising the amino acid sequence of SEQ ID NO: 9.

In some embodiments, a protein comprises two polypeptides, wherein each polypeptide comprises, from the N-terminus to the C-terminus, the first moiety comprising a variable domain having the sequence of SEQ ID NO: 13 and the second moiety comprising the amino acid sequence of SEQ ID NO: 9.

In some embodiments, a protein comprises two polypeptides, wherein each polypeptide comprises, from the N-terminus to the C-terminus, the third moiety comprising an Fc domain, the first moiety comprising an anti-C3bVHH and the second moiety comprising the domains 2-4 of MCP.

In some embodiments, a protein comprises two polypeptides, wherein each polypeptide comprises, from N-terminus to the C-terminus, the third moiety comprising SEQ ID NO: 31, the variable domain of the first moiety comprising a HCDR1 of SEQ ID NO: 16, a HCDR2 of SEQ ID NO: 17, and a HCDR3 of SEQ ID NO: 18, and the second moiety comprising the domains 2-4 of MCP.

In some embodiments, a protein comprises two polypeptides, wherein each polypeptide comprises, from N-terminus to the C-terminus, the third moiety comprising SEQ ID NO: 33, the variable domain of the first moiety comprising a HCDR1 of SEQ ID NO: 16, a HCDR2 of SEQ ID NO: 17, and a HCDR3 of SEQ ID NO: 18, and the second moiety comprising the domains 2-4 of MCP.

In some embodiments, a protein comprises two polypeptides, wherein each polypeptide comprises, from N-terminus to the C-terminus, the third moiety comprising SEQ ID NO: 36, the variable domain of the first moiety comprising a HCDR1 of SEQ ID NO: 16, a HCDR2 of SEQ ID NO: 17, and a HCDR3 of SEQ ID NO: 18, and the second moiety comprising the domains 2-4 of MCP.

In some embodiments, a protein comprises two polypeptides, wherein each polypeptide comprises, from N-terminus to the C-terminus, the third moiety comprising SEQ ID NO: 31, the variable domain of the first moiety comprising a HCDR1 of SEQ ID NO: 19, a HCDR2 of SEQ ID NO: 20, and a HCDR3 of SEQ ID NO: 18, and the second moiety comprising the domains 2-4 of MCP.

In some embodiments, a protein comprises two polypeptides, wherein each polypeptide comprises, from N-terminus to the C-terminus, the third moiety comprising SEQ ID NO: 33, the variable domain of the first moiety comprising a HCDR1 of SEQ ID NO: 19, a HCDR2 of SEQ ID NO: 20, and a HCDR3 of SEQ ID NO: 18, and the second moiety comprising the domains 2-4 of MCP.

In some embodiments, a protein comprises two polypeptides, wherein each polypeptide comprises, from N-terminus to the C-terminus, the third moiety comprising SEQ ID NO: 36, the variable domain of the first moiety comprising a HCDR1 of SEQ ID NO: 19, a HCDR2 of SEQ ID NO: 20, and a HCDR3 of SEQ ID NO: 18, and the second moiety comprising the domains 2-4 of MCP.

In some embodiments, a protein comprises two polypeptides, wherein each polypeptide comprises, from N-terminus to the C-terminus, the third moiety comprising SEQ ID NO: 31, the variable domain of the first moiety comprising a HCDR1 of SEQ ID NO: 16, a HCDR2 of SEQ ID NO: 26, and a HCDR3 of SEQ ID NO: 22, and the second moiety comprising the domains 2-4 of MCP.

In some embodiments, a protein comprises two polypeptides, wherein each polypeptide comprises, from N-terminus to the C-terminus, the third moiety comprising SEQ ID NO: 33, the variable domain of the first moiety comprising a HCDR1 of SEQ ID NO: 16, a HCDR2 of SEQ ID NO: 26, and a HCDR3 of SEQ ID NO: 22, and the second moiety comprising the domains 2-4 of MCP.

In some embodiments, a protein comprises two polypeptides, wherein each polypeptide comprises, from N-terminus to the C-terminus, the third moiety comprising SEQ ID NO: 36, the variable domain of the first moiety comprising a HCDR1 of SEQ ID NO: 16, a HCDR2 of SEQ ID NO: 26, and a HCDR3 of SEQ ID NO: 22, and the second moiety comprising the domains 2-4 of MCP.

In some embodiments, a protein comprises two polypeptides, wherein each polypeptide comprises, from N-terminus to the C-terminus, the third moiety comprising SEQ ID NO: 31, the variable domain of the first moiety comprising a HCDR1 of SEQ ID NO: 19, a HCDR2 of SEQ ID NO: 28, and a HCDR3 of SEQ ID NO: 27, and the second moiety comprising the domains 2-4 of MCP.

In some embodiments, a protein comprises two polypeptides, wherein each polypeptide comprises, from N-terminus to the C-terminus, the third moiety comprising SEQ ID NO: 33, the variable domain of the first moiety comprising a HCDR1 of SEQ ID NO: 19, a HCDR2 of SEQ ID NO: 28, and a HCDR3 of SEQ ID NO: 27, and the second moiety comprising the domains 2-4 of MCP.

In some embodiments, a protein comprises two polypeptides, wherein each polypeptide comprises, from N-terminus to the C-terminus, the third moiety comprising SEQ ID NO: 36, the variable domain of the first moiety comprising a HCDR1 of SEQ ID NO: 19, a HCDR2 of SEQ ID NO: 28, and a HCDR3 of SEQ ID NO: 27, and the second moiety comprising the domains 2-4 of MCP.

In some embodiments, a protein comprises two polypeptides, wherein each polypeptide comprises, from the N-terminus to the C-terminus, the third moiety comprising SEQ ID NO: 31, the first moiety comprising a variable domain having the sequence of SEQ ID NO: 10 and the second moiety comprising the amino acid sequence of SEQ ID NO: 9.

In some embodiments, a protein comprises two polypeptides, wherein each polypeptide comprises, from the N-terminus to the C-terminus, the third moiety comprising SEQ ID NO: 33, the first moiety comprising a variable domain having the sequence of SEQ ID NO: 10 and the second moiety comprising the amino acid sequence of SEQ ID NO: 9.

In some embodiments, a protein comprises two polypeptides, wherein each polypeptide comprises, from the N-terminus to the C-terminus, the third moiety comprising SEQ ID NO: 36, the first moiety comprising a variable domain having the sequence of SEQ ID NO: 10 and the second moiety comprising the amino acid sequence of SEQ ID NO: 9.

In some embodiments, a protein comprises two polypeptides, wherein each polypeptide comprises, from the N-terminus to the C-terminus, the third moiety comprising SEQ ID NO: 31, the first moiety comprising a variable domain having the sequence of SEQ ID NO: 13 and the second moiety comprising the amino acid sequence of SEQ ID NO: 9.

In some embodiments, a protein comprises two polypeptides, wherein each polypeptide comprises, from the N-terminus to the C-terminus, the third moiety comprising SEQ ID NO: 33, the first moiety comprising a variable domain having the sequence of SEQ ID NO: 13 and the second moiety comprising the amino acid sequence of SEQ ID NO: 9.

In some embodiments, a protein comprises two polypeptides, wherein each polypeptide comprises, from the N-terminus to the C-terminus, the third moiety comprising SEQ ID NO: 36, the first moiety comprising a variable domain having the sequence of SEQ ID NO: 13 and the second moiety comprising the amino acid sequence of SEQ ID NO: 9.

In some embodiments, a protein comprises two polypeptides, wherein each polypeptide comprises, from the N-terminus to the C-terminus, the third moiety comprising SEQ ID NO: 31, the first moiety comprising a variable domain having the sequence of SEQ ID NO: 10 and the second moiety comprising the amino acid sequence of SEQ ID NO: 15.

In some embodiments, a protein comprises two polypeptides, wherein each polypeptide comprises, from the N-terminus to the C-terminus, the third moiety comprising SEQ ID NO: 33, the first moiety comprising a variable domain having the sequence of SEQ ID NO: 10 and the second moiety comprising the amino acid sequence of SEQ ID NO: 15.

In some embodiments, a protein comprises two polypeptides, wherein each polypeptide comprises, from the N-terminus to the C-terminus, the third moiety comprising SEQ ID NO: 36, the first moiety comprising a variable domain having the sequence of SEQ ID NO: 10 and the second moiety comprising the amino acid sequence of SEQ ID NO: 15.

In some embodiments, a protein comprises two polypeptides, wherein each polypeptide comprises, from the N-terminus to the C-terminus, the third moiety comprising SEQ ID NO: 31, the first moiety comprising a variable domain having the sequence of SEQ ID NO: 13 and the second moiety comprising the amino acid sequence of SEQ ID NO: 15.

In some embodiments, a protein comprises two polypeptides, wherein each polypeptide comprises, from the N-terminus to the C-terminus, the third moiety comprising SEQ ID NO: 33, the first moiety comprising a variable domain having the sequence of SEQ ID NO: 13 and the second moiety comprising the amino acid sequence of SEQ ID NO: 15.

In some embodiments, a protein comprises two polypeptides, wherein each polypeptide comprises, from the N-terminus to the C-terminus, the third moiety comprising SEQ ID NO: 36, the first moiety comprising a variable domain having the sequence of SEQ ID NO: 13 and the second moiety comprising the amino acid sequence of SEQ ID NO: 15.

In some embodiments, a protein comprises two polypeptides, wherein each polypeptide comprises, from the N-terminus to the C-terminus, the third moiety comprising SEQ ID NO: 31, the first moiety comprising the sequence of SEQ ID NO: 55, and the second moiety comprising the amino acid sequence of SEQ ID NO: 9.

### Nucleic Acids Encoding Polypeptides

In one aspect, the present invention provides, among other things a nucleic acid encoding a polypeptide or a protein of the present disclosure.

In some embodiments, a nucleic acid encodes a polypeptide that is capable of binding to complement C3b (C3b), wherein the polypeptide comprises at least (A) a first moiety and (B) a second moiety, wherein the first moiety comprises a variable domain capable of binding to C3b, and the second moiety comprises an amino acid sequence of membrane cofactor protein (MCP) or a variant thereof.

In some embodiments, a nucleic acid encodes a polypeptide comprising, from the N-terminus to the C-terminus, the first moiety comprising an anti-C3bVHH and the second moiety comprising the domains 2-4 of MCP.

In some embodiments, a nucleic acid encodes a polypeptide comprising, from N-terminus to the C-terminus, the variable domain of the first moiety comprising a HCDR1 of SEQ ID NO: 16, a HCDR2 of SEQ ID NO: 17, and a HCDR3 of SEQ ID NO: 18, and the second moiety comprising the domains 2-4 of MCP.

In some embodiments, a nucleic acid encodes a polypeptide comprising, from N-terminus to the C-terminus, the variable domain of the first moiety comprising a HCDR1 of SEQ ID NO: 19, a HCDR2 of SEQ ID NO: 20, and a HCDR3 of SEQ ID NO: 18, and the second moiety comprising the domains 2-4 of MCP.

In some embodiments, a nucleic acid encodes a polypeptide comprising, from N-terminus to the C-terminus, the variable domain of the first moiety comprising a HCDR1 of SEQ ID NO: 16, a HCDR2 of SEQ ID NO: 26, and a HCDR3 of SEQ ID NO: 22, and the second moiety comprising the domains 2-4 of MCP.

In some embodiments, a nucleic acid encodes a polypeptide comprising, from N-terminus to the C-terminus, the variable domain of the first moiety comprising a HCDR1 of SEQ ID NO: 19, a HCDR2 of SEQ ID NO: 28, and a HCDR3 of SEQ ID NO: 27, and the second moiety comprising the domains 2-4 of MCP.

In some embodiments, a nucleic acid encodes a polypeptide comprising, from the N-terminus to the C-terminus, the first moiety comprising a variable domain having the sequence of SEQ ID NO: 10 and the second moiety comprising the amino acid sequence of SEQ ID NO: 9.

In some embodiments, a nucleic acid encodes a polypeptide comprising, from the N-terminus to the C-terminus, the first moiety comprising a variable domain having the sequence of SEQ ID NO: 13 and the second moiety comprising the amino acid sequence of SEQ ID NO: 9.

In some embodiments, a nucleic acid is a DNA, a CDNA or an RNA. In some embodiments, a nucleic acid is a cDNA. In some embodiments, a nucleic acid is an RNA. In some embodiments, a nucleic acid is a messenger RNA (mRNA).

In some embodiments, the present invention provides, a mRNA encoding any of the polypeptides disclosed herein.

mRNA Encoding a Polypeptide Capable of Binding to C3b

In some embodiments, the present invention provides an mRNA encoding a nucleic acid encodes a polypeptide that is capable of binding to complement C3b (C3b), wherein the polypeptide comprises at least (A) a first moiety and (B) a second moiety, wherein the first moiety comprises a variable domain capable of binding to C3b, and the second moiety comprises an amino acid sequence of membrane cofactor protein (MCP) or a variant thereof.

In some embodiments, an mRNA encoding a polypeptide that is capable of binding to C3b is codon optimized. In some embodiments, the present invention provides methods and compositions for delivering codon optimized mRNA encoding a polypeptide that is capable of binding to C3b to a subject for the treatment of complement mediated disease. A suitable codon optimized mRNA encodes any full length, fragment or portion of a polypeptide that is capable of binding to C3b, for example, a first moiety, a second moiety or both.

Synthesis of mRNA

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

Exemplary mRNA Construct

### Construct Design:

5′ UTR-mRNA encoding an antibody or a fragment thereof-3′ UTR

### Codon Optimization

According to an increasing amount of research, mRNAs contain numerous layers of information that overlap the amino acid code. Traditionally, codon optimization has been used to remove rare codons which were thought to be rate-limiting for protein expression. While fast growing bacteria and yeast both exhibit strong codon bias in highly expressed genes, higher eukaryotes exhibit much less codon bias, making it more difficult to discern codons that may be rate-limiting. In addition, it has been found that codon bias per se does not necessarily yield high expression but requires other features.

For example, rare codons have been implicated in slowing translation and forming pause sites, which may be required for correct protein folding. Therefore, variations in codon usage may provide a mechanism to fine-tune the temporal pattern of elongation and thus increase the time available for a protein to take on its correct confirmation. Codon optimization can interfere with this fine-tuning mechanism, resulting in less efficient protein translation or an increased amount of incorrectly folded proteins. Similarly, codon optimization may disrupt the normal patterns of cognate and wobble tRNA usage, thereby affecting protein structure and function because wobble-dependent slowing of elongation may likewise have been selected as a mechanism for achieving correct protein folding.

Various methods of performing codon optimization are known in the art, however, each has significant drawbacks and limitations from a computational and/or therapeutic point of view. In particular, known methods of codon optimization often involve, for each amino acid, replacing every codon with the codon having the highest usage for that amino acid, such that the “optimized” sequence contains only one codon encoding each amino acid (so may be referred to as a one-to-one sequence). The increase in expression is not limited to cell cultures of mammalian cells but was also observed in vivo in a mouse model. It is expected that the observed improvement in expression of the codon-optimized coding sequence will result in an improved, more cost-effective mRNA replacement therapy for patients in need thereof, because it does not require the use of modified nucleotides for the preparation of the mRNA and allows treatment with a reduced dose and/or at extended dosing intervals.

In some embodiments, codon-optimized mRNA is produced in accordance with methods known in the art.

### Nucleotides

Various naturally-occurring or modified nucleosides may be used to product mRNA according to the present invention. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“ψU”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

### Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published US Application No. US 2016/0032356 and U.S. Provisional Application 62/464,327, filed Feb. 27, 2017, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

### Delivery

### Protein Administration

In some embodiments, a polypeptide that is capable of binding to C3b is administered to a subject in need of.

In some embodiments, nanoparticles, liposomes, virus-like particles (VLPs), exosomes, and engineered bacteria are used to deliver a polypeptide or the protein of the present invention.

In some embodiments, a polypeptide that is capable of binding to C3b is encapsulated in a nanoparticle. In some embodiments, a polypeptide that is capable of binding to C3b is encapsulated in a lipid nanoparticle. In some embodiments, a polypeptide that is capable of binding to C3b is encapsulated in a liposome. In some embodiments, a polypeptide that is capable of binding to C3b is encapsulated in a VLP. In some embodiments, a polypeptide that is capable of binding to C3b is encapsulated in an exosome. In some embodiments, a polypeptide that is capable of binding to C3b is encapsulated in an engineered bacteria.

In some embodiments, a protein that is capable of binding to C3b is encapsulated in a nanoparticle. In some embodiments, a protein that is capable of binding to C3b is encapsulated in a lipid nanoparticle. In some embodiments, a protein that is capable of binding to C3b is encapsulated in a liposome. In some embodiments, a protein that is capable of binding to C3b is encapsulated in a VLP. In some embodiments, a protein that is capable of binding to C3b is encapsulated in an exosome. In some embodiments, a protein that is capable of binding to C3b is encapsulated in an engineered bacteria.

### Viral Vector and Gene Therapy

In some embodiments, a pharmaceutical composition comprising nucleic acids encoding a polypeptide that is capable of binding to C3b is used to treat subjects in need thereof. In some embodiments, a pharmaceutical composition comprising a rAAV vector described herein is used to treat subjects in need thereof. The pharmaceutical composition containing a rAAV vector or particle of the invention contains a pharmaceutically acceptable excipient, diluent, or carrier. Examples of suitable pharmaceutical carriers are well known in the art and include phosphate buffered saline solutions, water, emulsions, such as oil/water emulsions, various types of wetting agents, sterile solutions and the like. The pharmaceutical composition can be in a lyophilized form. Such carriers can be formulated by conventional methods and are administered to the subject at a therapeutically effective amount.

The rAAV vector is administered to a subject in need thereof via a suitable route. In some embodiments, the rAAV vector is administered by intravenous, intraperitoneal, subcutaneous, or intradermal routes. In one embodiment, the rAAV vector is administered intravenously. In embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle or an inorganic nanoparticle.

In some embodiments, a rAAV is pseudotyped. A pseudotyped rAAV is an infectious virus comprising any combination of an AAV capsid protein and a rAAV genome. Pseudotyped rAAV are useful to alter the tissue or cell specificity of rAAV, and may be employed alone or in conjunction with non-pseudotyped rAAV to transfer one or more genes to a cell, e.g., a mammalian cell. For example, pseudotyped rAAV may be employed subsequent to administration with non-pseudotyped rAAV in a mammal which has developed an immune response to the non-pseudotyped rAAV. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 ITRs, each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector of the invention may be homologous, i.e., from the same serotype, heterologous, i.e., from different serotypes, or chimeric, i.e., an ITR which has ITR sequences from more than one AAV serotype.

In some embodiments, the rAAV vector is an AAV1, AAV2, AAV3, AAV4, AAV5, AAV6, AAV7, AAV8, AAV9, AAV10, or AAV11 vector. In some embodiments, the rAAV vector is AAV1. In some embodiments, the rAAV vector is AAV2. In some embodiments, the rAAV vector is AAV3. In some embodiments, the rAAV vector is AAV4. In some embodiments, the rAAV vector is AAV5. In some embodiments, the rAAV vector is AAV6. In some embodiments, the rAAV vector is AAV7. In some embodiments, the rAAV vector is AAV8. In some embodiments, the rAAV vector is AAV9. In some embodiments, the rAAV vector is AAV10. In some embodiments, the rAAV vector is AAV11. In some embodiments, the rAAV vector is sequence optimized. In some embodiments, the rAAV capsid is modified. For example, in some embodiments, the rAAV8 capsid is modified.

mRNA Delivery

According to the present invention, mRNA encoding a polypeptide that is capable of binding to C3b as described herein may be delivered as naked RNA (unpackaged) or via delivery vehicles. As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

### Liposomal Delivery Vehicles

In some embodiments, a suitable delivery vehicle is a liposomal delivery vehicle, e.g., a lipid nanoparticle (LNP). As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired mRNA to a target cell or tissue. In some embodiments, a nanoparticle delivery vehicle is a liposome. In some embodiments, a liposome comprises one or more cationic lipids, one or more non-cationic lipids, one or more cholesterol-based lipids and one or more PEG-modified lipids.

Treatment with C3b Inhibitor Polypeptides or Protein

In some embodiments, the present invention provides, among other things, a method of treating complement mediated disorders (e.g., C3 glomerulopathy) by administering a polypeptide that is capable of binding to C3b.

In some embodiments, the present invention provides, among other things, a method of treating complement medicated disorder in a subject comprising administering to the subject a polypeptide that is capable of binding to C3b at a therapeutically effective dose and an administration interval for a treatment period sufficient to improve, stabilize or reduce one or more symptoms of the said disorder. In some embodiments, the present invention provides, among other things, a method of treating complement medicated disorder in a subject comprising administering to the subject a composition comprising a nucleic acid encoding a polypeptide that is capable of binding to C3b at a therapeutically effective dose and an administration interval for a treatment period sufficient to improve, stabilize or reduce one or more symptoms of the said disorder. In some embodiments, the present invention provides, among other things, a method of treating complement medicated disorder in a subject comprising administering to the subject a composition comprising mRNA encoding a polypeptide that is capable of binding to C3b at a therapeutically effective dose and an administration interval for a treatment period sufficient to improve, stabilize or reduce one or more symptoms of the said disorder.

### Complement Mediated Disorders

Many autoimmune diseases are characterized by generation of autoantibodies that bind to host proteins or deposit within tissues as a component of immune complexes. The autoantibodies can activate the complement system, which can mediate tissue damage and trigger systemic inflammation. Complement inhibitory drugs may, therefore, be beneficial across a large number of different autoimmune diseases.

C3 glomerulopathy (C3G) is a rare disease that is characterized by accumulation of complement factors in the glomeruli due to overactivation and abnormal regulation of the alternative pathway (AP) of complement. It is an ultra-rare disease with an incidence of approximately 1-3 per million per year. C3 glomerulopathy is an umbrella term for a spectrum of related diseases or disorders characterized by accumulation of the C3 component of complement in renal tissue. Abnormal control of the AP of complement may be due to acquired or genetic abnormalities of the complement regulatory proteins. The deposition of complement factors drives glomerular inflammation, resulting in a proliferative glomerulonephritis. The most common diseases under the C3G umbrella are C3 Glomerulonephritis (C3GN), characterized by mesangial, subendothelial, and intramembranous deposits, and Dense Deposit Disease (DDD), characterized by dense deposits along the glomerular and tubular basement membranes. Presentation of disease ranges from asymptomatic hematuria and proteinuria to an acute presentation with the classic signs and symptoms of glomerulonephritis. C3 glomerulopathy is driven by acquired factors (anti-C3 and C5 convertase autoantibodies) and genetic mutations in complement-related genes. These autoantibodies drive complement dysregulation by increasing the half-life of these vital but normally short-lived enzymes. Genetic mutations in Factor H, Complement Factor H related (CFHR) proteins, C3 can lead to impaired regulation of C3 convertase.

Other diseases associated with AP overactivation includes Paroxysmal Nocturnal Hemoglobinuria with extravascular hemolysis, which is a rare, acquired, blood disorder where RBCs are targeted by C3b rather than MAC. In patients with Paroxysmal Nocturnal Hemoglobinuria, C5 inhibitors do not work, and it remains a major unmet medical need. Geographic Atrophy (GA) is another complication that is associated with AP overactivation. Geographic atrophy is an advanced manifestation of Age-related Macular Degeneration (AMD) characterized by progressive and irreversible loss of the retinal pigment epithelium and photoreceptors, a major cause of legal blindness. 98% of wet AMD patients develop GA despite anti-VEGF treatment.

In one aspect, the present invention provides a method of treating a disease or disorder, said method comprises administering a therapeutically effective amount of a polypeptide that is capable of binding to C3b to a subject in need thereof. In some embodiments, the disease or disorder is complement mediated disorder. In some embodiments, the disease or disorder is C3 glomerulopathy. In some embodiments, the disease or disorder is associated with C3 glomerulopathy. In some embodiments, the disease or disorder is dense deposit disease. In some embodiments, the disease or disorder is C3 glomerulonephritis (C3GN). In some embodiments, the disease or disorder is disorder is characterized by accumulation of the C3 component of complement in renal tissue. In some embodiments, the disease or disorder is hematuria. In some embodiments, the disease or disorder is proteinuria. In some embodiments, the disease or disorder is Acute Kidney Injury (AKI). In some embodiments, the disease or disorder is Chronic Kidney Disease (CKD). In some embodiments, the disease or disorder is associated with AP overactivation. In some embodiments, the disease or disorder is Paroxysmal Nocturnal Hemoglobinuria. In some embodiments, the disease or disorder is Geographic Atrophy (GA). In some embodiments, the disease or disorder is autoimmune hemolytic anemia (AIHA). In some embodiments, the disease or disorder is Age-related Macular Degeneration (AMD). In some embodiments, the disease or disorder is wet Age-related Macular Degeneration (wAMD). In some embodiments, the disease or disorder is Passive Heymann Nephritis (PHN). In some embodiments, the disease or disorder is rheumatoid arthritis (RA). In some embodiments, the disease or disorder is collagen-antibody induced arthritis (CAIA). In some embodiments, the disease or disorder is Membranous Nephropathy (MN). In some embodiments, the disease or disorder is Diabetic Kidney Disease (DKD). In some embodiments, the disease or disorder is Sepsis.

In some embodiments, the disease or disorder is C3 glomerulopathy (C3G)/Immune complex membranoproliferative glomerulonephritis (IC-MPGN), Atypical hemolytic uremic syndrome (aHUS), Diabetic nephropathy (DN) specifically in type 1 diabetes (T1D), Chronic antibody mediated rejection (cAMR), Chronic inflammatory demyelinating polyneuropathy (CIDP), Dermatomyositis (DM), or Paroxysmal nocturnal hemoglobinuria (PNH).

In some embodiments, the disease or disorder is Chronic antibody mediated rejection (cAMR), chronic inflammatory demyelinating polyneuropathy (CIDP), or Dermatomyositis (DM).

In some embodiments, the disease or disorder is caused by genetic mutation in Factor H. In some embodiments, the disease or disorder is caused by genetic mutation in CFHR proteins. In some embodiments, the disease or disorder is caused by genetic mutation in C43. In some embodiments, the disease is driven by acquired factors. In some embodiments, the acquired factors are anti-C3 autoantibodies. In some embodiments, the acquired factors are C5 convertase autoantibodies.

### EMBODIMENTS

Also disclosed with regard to the above-described subject matter is the following:
[A1]A polypeptide that is capable of binding to complement C3b (C3b), wherein the polypeptide comprises at least (A) a first moiety and (B) a second moiety, wherein the first moiety comprises a variable domain capable of binding to C3b, and the second moiety comprises an amino acid sequence of membrane cofactor protein (MCP) or a variant thereof.
[A2] The polypeptide of A1, wherein the first moiety comprises heavy complementary-determining region 1 (HCDR1), a heavy complementary-determining region 2 (HCDR2) and a heavy complementary-determining region 3 (HCDR3).
[A3] The polypeptide of A1 or A2, wherein the first moiety is single domain antibody.
[A4] The polypeptide of any one of A1-A3, wherein the first moiety is VHH.
[A5] The polypeptide of any one of A1-A4, wherein the second moiety comprises complement control protein (CCP) modules of MCP or a variant thereof.
[A6] The polypeptide of any one of A1-A5, wherein the second moiety comprises domains 2-4 of MCP (corresponds to CCP modules 2-4 of MCP) or a variant thereof. [A7] The polypeptide of any one of A1-A6, wherein the polypeptide binds to human C3(H₂O).
[A8] The polypeptide of any one of A1-A7, wherein the polypeptide inhibits C3 deposition.
[A9] The polypeptide of any one of A1-A8, wherein the polypeptide inhibits C3 deposition in normal cynomolgus monkey serum (NCS).
[A10] The polypeptide of any one of A1-A9, wherein the polypeptide inhibits C3 deposition in normal human serum (NHS).
[A11] The polypeptide of any one of A1-A10, wherein the polypeptide inhibits MAC deposition.
[A12] The polypeptide of any one of A1-A11, wherein the polypeptide does not cause C3 consumption.
[A13] The polypeptide of any one of A1-A12, wherein the polypeptide does not cause C3 consumption in normal cynomolgus monkey serum (NCS).
[A14] The polypeptide of any one of A1-A13, wherein the polypeptide binds to human C3b with greater affinity than to human C3.
[A15] The polypeptide of any one of A1-A14, wherein the first moiety and the second moiety bind to a single molecule of human C3b at the same time.
[A16] The polypeptide of any one of A1-A15, wherein the first moiety and the second moiety bind to a single molecule of human C3(H₂O) at the same time.
[A17] The polypeptide of any one of A1-A16, wherein the polypeptide prevents the association of human factor B with human C3b.
[A18] The polypeptide of any one of A1-A17, wherein the polypeptide prevents the association of human factor B with human C3(H₂O).
[A19] The polypeptide of any one of A1-A18, wherein the polypeptide hinders formation of human C3 convertase.
[A20] The polypeptide of any one of A1-A19, wherein the polypeptide recruits human factor I to human C3b.
[A21] The polypeptide of any one of A1-A20, wherein the polypeptide recruits human factor I to human C3(H₂O).
[A22] The polypeptide of any one of A1-A21, wherein the polypeptide facilitates proteolytic cleavage of human C3b.
[A23] The polypeptide of any one of A1-A22, wherein the polypeptide facilitates proteolytic cleavage of human C3(H₂O).
[A24] The polypeptide of any one of A1-A23, wherein the variable domain of the first moiety comprises a heavy complementary-determining region 1 (HCDR1), a heavy complementary-determining region 2 (HCDR2) and a heavy complementary-determining region 3 (HCDR3), wherein the HCDR1, HCDR2 and HCDR3 comprise the sequence of SEQ ID NO: 16, 17, and 18 respectively.
[A25] The polypeptide of any one of A1-A24, wherein the first moiety comprises the sequence of SEQ ID NO: 10.
[A26] The polypeptide of any one of A1-A25, wherein the variable domain of the first moiety comprises a HCDR1, a HCDR2 and a HCDR3, wherein the HCDR1, HCDR2 and HCDR3 comprises the sequence of SEQ ID NO: 16, 26, and 22 respectively.
[A27] The polypeptide of any one of A1-A26, wherein the first moiety comprises the sequence of SEQ ID NO: 13.
[A28] The polypeptide of any one of A1-A27, wherein the second moiety has at least 90% sequence identity to the sequence of SEQ ID NO: 14.
[A29] The polypeptide of any one of A1-A28, wherein the second moiety has at least 90% sequence identity to the sequence of SEQ ID NO: 9.
[A30] The polypeptide of any one of A1-A29, wherein the second moiety comprises a mutation of S177R to the sequence of SEQ ID NO: 9.
[A31] The polypeptide of any one of A1-A30, wherein the second moiety comprises the sequence of SEQ ID NO: 15.
[A32] The polypeptide of any one of A1-A31, wherein the first moiety is directly or indirectly linked to the second moiety.
[A33] The polypeptide of any one of A1-A32, wherein the first moiety is linked to the second moiety via a linker (Linker 1).
[A34] The polypeptide of any one of A1-A33, wherein the C-terminus of the first moiety is linked to the N-terminus of the second moiety.
[A35] The polypeptide of any one of A1-A34 comprising, from the N-terminus to the C-terminus, the first moiety comprising the sequence of SEQ ID NO: 10 linked to the second moiety comprising the sequence of SEQ ID NO: 9.
[A36] The polypeptide of any one of A1-A35 comprising, from the N-terminus to the C-terminus, the first moiety comprising the sequence of SEQ ID NO: 13 linked to the second moiety comprising the sequence of SEQ ID NO: 9.
[A37] The polypeptide of any one of A1-A36 comprising, from the N-terminus to the C-terminus, the first moiety comprising the sequence of SEQ ID NO: 10 linked to the second moiety comprising the sequence of SEQ ID NO: 15.
[A38] The polypeptide of any one of A33-A37, wherein Linker 1 is 20 amino acids or less.
[A39] The polypeptide of any one of A38, wherein Linker 1 comprises GGGGS.
[A40] The polypeptide of any one of A39, wherein Linker 1 comprises GGGGSGGGGSGGGGS.
[A41] The polypeptide of any one of A1-A40, wherein the polypeptide comprises the sequence of SEQ ID NO: 5 or SEQ ID NO: 8.
[A42] The polypeptide of any one of A1-A41, further comprises (C) a third moiety comprising a half-life extension domain.
[A43] The polypeptide of any one of A1-A42, wherein the third moiety comprises an Fc domain.
[A44] The polypeptide of any one of A1-A43, wherein the Fc domain is derived from IgG1, IgG2, or IgG4.
[A45] The polypeptide of any one of A44, wherein the Fc domain is derived from IgG4.
[A46] The polypeptide of any one of A45, wherein the Fc domain is derived from IgG1.
[A47] The polypeptide of any one of A43-A46, wherein the Fc domain comprises an amino acid modification of T307Q, Q311V and A378V.
[A48] The polypeptide of any one of A43-A47, wherein the Fc domain comprises an amino acid modification of F234V, L235E and D265G.
[A49] The polypeptide of any one of A43-A48, wherein the Fc domain comprises an amino acid modification of S228P.
[A50] The polypeptide of any one of A43-A49, wherein the N-terminus of the first moiety is linked to the C-terminus of the third moiety.
[A51] The polypeptide of any one of A43-A50, wherein the polypeptide comprises, from the N-terminus to the C-terminus, the third moiety comprising the Fc domain having the amino acid modifications of a)T307Q, Q311V and A378V; b) F234V, L235E and D265G; and c) S228P, the first moiety comprising the sequence of SEQ ID NO: 10, and the second moiety comprising the sequence of SEQ ID NO: 9.
[A52] The polypeptide of any one of A43-A50, wherein the polypeptide comprises, from the N-terminus to the C-terminus, the third moiety comprising the Fc domain having the amino acid modifications of a)T307Q, Q311V and A378V; and b) F234V, L235E and D265G, the first moiety comprising the sequence of SEQ ID NO: 10, and the second moiety comprising the sequence of SEQ ID NO: 9.
[A53] The polypeptide of any one of A43-A50, wherein the polypeptide comprises, from the N-terminus to the C-terminus, the third moiety comprising the Fc domain having the amino acid modifications of a)T307Q, Q311V and A378V; b) F234V, L235E and D265G; and c) S228P, the first moiety comprising the sequence of SEQ ID NO: 10, the second moiety comprising the sequence of SEQ ID NO: 15.
[A54] The polypeptide of any one of A43-A50, wherein the polypeptide comprises, from the N-terminus to the C-terminus, the third moiety comprising the Fc domain having the amino acid modifications of a) F234V, L235E and D265G and b) S228P, the first moiety comprising the sequence of SEQ ID NO: 10, the second moiety comprising the sequence of SEQ ID NO: 9.
[A55] The polypeptide of any one of A43-A50, comprises, from the N-terminus to the C-terminus, the third moiety comprising the Fc domain having the amino acid modification of S228P, the first moiety comprising the sequence of SEQ ID NO: 13, and the second moiety comprising the sequence of SEQ ID NO: 9.
[A56] The polypeptide of any one of A43-A50, comprises, from the N-terminus to the C-terminus, the third moiety comprising the Fc domain having the amino acid modifications of a) F234V, L235E and D265G and b) S228P, the first moiety comprising the sequence of SEQ ID NO: 13, and the second moiety comprising the sequence of SEQ ID NO: 9.
[A57] The polypeptide of any one of A43-A50, comprises, from the N-terminus to the C-terminus, the third moiety comprising the Fc domain having the amino acid modifications of a)T307Q, Q311V and A378V; b) F234V, L235E and D265G; and c) S228P, the first moiety comprising the sequence of SEQ ID NO: 13, and the second moiety comprising the sequence of SEQ ID NO: 9.
[A58] The polypeptide of any one of A43-A50, comprises, from the N-terminus to the C-terminus, the third moiety comprising the Fc domain having the amino acid modification of S228P, the first moiety comprising the sequence of SEQ ID NO: 10, and the second moiety comprising the sequence of SEQ ID NO: 9.
[A59} The polypeptide of any one of A43-A50, comprises, from the N-terminus to the C-terminus, the third moiety comprising the Fc domain having the amino acid modifications of a) F234V, L235E and D265G and b) S228P, the first moiety comprising the sequence of SEQ ID NO: 55, and the second moiety comprising the sequence of SEQ ID NO: 9.
[A60] The polypeptide of any one of A43-A49, wherein the C-terminus of the second moiety is linked to the N-terminus of the third moiety.
[A61] The polypeptide of any one of A43-A49 and 58, comprises from the N-terminus to the C-terminus, the first moiety comprising the sequence of SEQ ID NO: 13, the second moiety comprising the sequence of SEQ ID NO: 9 and the third moiety comprising the Fc domain having the amino acid modifications of a)T307Q, Q311V and A378V; b) F234V, L235E and D265G; and c) S228P.
[A62] The polypeptide of any one of A43-A49, comprises from the N-terminus to the C-terminus, the first moiety comprising the sequence of SEQ ID NO: 13, the second moiety comprising the sequence of SEQ ID NO: 9 and the third moiety comprising the Fc domain having the amino acid modification of S228P.
[A63} The polypeptide of any one of A43-A49, comprises from the N-terminus to the C-terminus, the first moiety comprising the sequence of SEQ ID NO: 10, the second moiety comprising the sequence of SEQ ID NO: 9 and the third moiety comprising the Fc domain having the amino acid modifications a)T307Q, Q311V and A378V; b) F234V, L235E and D265G; and c) S228P.
[A64] The polypeptide of any one of A43-A63, wherein the first moiety and the third moiety are linked by a linker (Linker 2), wherein Linker 2 is 5-10 amino acids or less.
[A65] The polypeptide of any one of A42-A64, wherein the second moiety and the third moiety are linked by a linker (Linker 3), wherein Linker 3 is 5-10 amino acids or less.
[A66] The polypeptide of any one of A56 or A65, wherein Linker 2 or Linker 3 is GGGGGS (SEQ ID NO: 41).
[A67] The polypeptide of any one of A1-A66, wherein the sequence of the polypeptide is any one of SEQ ID NOs: 3, 4, 6, 7, 8, 43-54.
[B1]A protein comprising two polypeptides, wherein each polypeptide is a polypeptide of any one of A1-A59.
[B2] The protein of B1, wherein the protein is a homodimer.
[B3] The protein of B1, wherein the protein is a heterodimer.
[B4] The protein of any one of B1-B3, wherein the protein binds to human C3(H₂O).
[B5] The protein of any one of B1-B4, wherein the protein binds to human C3b with KD of 10 nM or less.
[B6] The protein of any one of B1-B5, wherein the protein binds to human C3b with KD of 5 nM or less.
[B7] The protein of any one of B1-B6, wherein the protein binds to human C3b with KD of 1 nM or less.
[B8] The protein of any one of B1-B7, wherein the protein binds to human C3b with KD of 0.5 nM or less.
[B9] The protein of any one of B1-B8, wherein the protein binds to human C3b with KD of 0.1 nM or less.
[B10] The protein of any one of B1-B9, wherein the protein binds to human C3b with KD of 0.05 nM or less.
[B11] The protein of any one of B1-B10, wherein the protein binds to human C3b with KD of 0.03 nM or less.
[B12] The protein of any one of B1-B11, wherein the protein binds to human C3b with KD of 0.001 nM or more.
[B13] The protein of any one of B1-B12, wherein the protein binds to human C3b with KD of 0.005 nM or more.
[B14] The protein of any one of B1-B13, wherein the protein binds to human C3b with KD of 0.01 nM or more.
[B15] The protein of any one of B1-B14, wherein the protein binds to human C3 with KD of 10 nM or less.
[B16] The protein of any one of B1-B15, wherein the protein binds to human C3(H₂O) with KD of 5 nM or less.
[B17] The protein of any one of B1-B16, wherein the protein binds to human C3(H₂O) with KD of 1 nM or less.
[B18] The protein of any one of B1-B17, wherein the protein binds to human C3(H₂O) with KD of 0.5 nM or less.
[B19] The protein of any one of B1-B18, wherein the protein binds to human C3(H₂O) with KD of 0.1 nM or less.
[B20] The protein of any one of B1-B19, wherein the protein binds to human C3(H₂O) with KD of 0.05 nM or less.
[B21] The protein of any one of B1-B20, wherein the protein binds to human C3(H₂O) with KD of 0.03 nM or less.
[B22] The protein of any one of B1-B21, wherein the protein binds to human C3(H₂O) with KD of 0.001 nM or more.
[B23] The protein of any one of B1-B22, wherein the protein binds to human C3(H₂O) with KD of 0.005 nM or more.
[B24] The protein of any one of B1-B23, wherein the protein binds to human C3(H₂O) with KD of 0.01 nM or more.
[B25] The protein of any one of B5-B24, wherein the KD is measured in accordance with the method described in Example 1.
[B26] The protein of any one of B1-B25, wherein the protein inhibits C3 deposition.
[B27] The protein of any one of B1-B26, wherein the protein inhibits C3 deposition with IC50 of 10 μm or less.
[B28] The protein of any one of B1-B27, wherein the protein inhibits C3 deposition with IC50 of 5 μm or less.
[B29] The protein of any one of B1-B28, wherein the protein inhibits C3 deposition with IC50 of 1 μm or less.
[B30] The protein of any one of B1-B29, wherein the protein inhibits C3 deposition with IC50 of 0.8 μm or less.
[B31] The protein of any one of B1-B30, wherein the protein inhibits C3 deposition with IC50 of 0.5 μm or less.
[B32] The protein of any one of B1-B31, wherein the protein inhibits C3 deposition with IC50 of 0.05 μm or more.
[B33] The protein of any one of B1-B32, wherein the protein inhibits C3 deposition with IC50 of 0.1 μm or more.
[B34] The protein of any one of B1-B33, wherein the protein inhibits C3 deposition with IC50 of 0.3 μm or more.
[B35] The protein of any one of B26-B34, wherein the IC50 is measured in accordance with the method described in Example 4.
[B36] The protein of any one of B1-B35, wherein the protein inhibits C3 deposition in normal cynomolgus monkey serum (NCS).
[B37] The protein of any one of B1-B36, wherein the protein inhibits C3 deposition in normal human serum (NHS).
[B38] The protein of any one of B1-B37, wherein the protein inhibits MAC deposition.
[B39] The protein of any one of B1-B38, wherein the protein inhibits MAC deposition with IC50 of 0.1 μm or less.
[B40] The protein of any one of B1-B39, wherein the protein inhibits MAC deposition with IC50 of 0.7 μm or less.
[B41] The protein of any one of B1-B40, wherein the protein inhibits MAC deposition with IC50 of 0.5 μm or less.
[B42] The protein of any one of B1-B41, wherein the protein inhibits MAC deposition with IC50 of 0.3 μm or less.
[B43] The protein of any one of B1-B42, wherein the protein inhibits MAC deposition with IC50 of 0.1 nm or more.
[B44] The protein of any one of B1-B43, wherein the protein inhibits MAC deposition with IC50 of 0.5 nm or more.
[B45] The protein of any one of B1-B44, wherein the protein inhibits MAC deposition with IC50 of 1 nm or more.
[B46] The protein of any one of B1-B45, wherein the protein inhibits MAC deposition with IC50 of 10 nm or more.
[B47] The protein of any one of B39-B46, wherein the IC50 is measured in accordance with the method described in Example 4.
[B48] The protein of any one of B1-B47, wherein the protein inhibits MAC deposition in normal cynomolgus monkey serum (NCS).
[B49] The protein of any one of B1-B48, wherein the protein inhibits MAC deposition in normal human serum (NHS).
[B50] The protein of any one of B1-B49, wherein the protein does not cause C3 consumption.
[B51] The protein of any one of B1-B50, wherein the protein does not cause C3 consumption in normal cynomolgus monkey serum (NCS).
[B52] The protein of any one of B1-B52, wherein the protein binds to human C3b with greater affinity than to human C3.
[B53] The protein of any one of B1-B52, wherein the first moiety and the second moiety bind to a single molecule of human C3b at the same time.
[B54] The protein of any one of B1-B53, wherein the first moiety and the second moiety bind to a single molecule of human C3(H₂O) at the same time.
[B55] The protein of any one of B1-B54, wherein the protein prevents the association of human factor B with human C3b.
[B56] The protein of any one of B1-B55, wherein the protein prevents the association of human factor B with human C3(H₂O).
[B57] The protein of any one of B1-B56, wherein the protein hinders formation of human C3 convertase.
[B58] The protein of any one of B1-B57, wherein the protein recruits human factor I to human C3b.
[B59] The protein of any one of B1-B58, wherein the protein recruits human factor I to human C3(H₂O).
[B60] The protein of any one of B1-B59, wherein the protein facilitates proteolytic cleavage of human C3b.
[B61] The protein of any one of B1-B60, wherein the protein facilitates proteolytic cleavage of human C3(H₂O).
[B62]A protein comprising two identical polypeptides, wherein each polypeptide comprises. a VHH that binds to C3b comprising a HCDR1 comprising NYHMG (SEQ ID NO: 16), a HCDR2 comprising TIIRTGETIYYADSVKG (SEQ ID NO:17). and a HCDR3 comprising ATSGWNIKTVTPYEY (SEQ ID NO:18) and an amino acid sequence of Membrane Cofactor Protein (MCP) comprising SEQ ID NO: 9.
[B62] The protein of B62, wherein each polypeptide further comprises a Fc domain comprising amino acid substitutions of S228P, F234V, L235E, D265G, T307Q, Q311V and A378V, according to EU index.
[B63] The protein of B61 or B62, wherein each polypeptide comprises from the N-terminus to the C-terminus, the Fc domain, the VHH and the sequence of MCP.
[B64] The therapeutic protein of any of B61-B63, wherein the VHH comprises an amino acid sequence of SEQ ID NO: 10.
[B65] The protein of any of B61-B64, wherein the Fc domain is a human IgG4 Fc domain.
[B66] The protein of any of B61-B65, wherein the Fc domain comprises the amino acid sequence of SEQ ID NO: 33.
[B67] The protein of any of B61-B66, wherein the Fc domain is linked to the VHH via a first peptide linker and the VHH is linked to the sequence of MCP via a second peptide linker.
[B68] The protein of B67, wherein the first peptide linker comprises GGGGGS (SEQ ID NO: 41) and the second peptide linker comprises GGGSGGGSGGGS (SEQ ID NO: 39).
[B69] The therapeutic protein of any of B61-B68, wherein each polypeptide comprises the Fc domain of SEQ ID NO. 33, the VHH domain of SEQ ID NO. 10 and the sequence of MCP of SEQ ID NO: 9.
[B70]A protein comprising two identical amino acid sequences of SEQ ID NO: 3.
[C1]A nucleic acid encoding the polypeptide of any one of A1-A59 or the protein of any one of B1-B61.
[C2]A vector comprising the nucleic acid of C1.
[C3] The vector of C2, wherein the vector is a viral vector.
[C4] The vector of C3, wherein the viral vector is an adeno-associate virus (AAV) vector.
[C5] The vector of C4, wherein the AAV vector is selected from AAV1, AAV2, AAV3, AAV4, AAV5, AAV6, AAV7, AAV8, AAV9, AAV10, AAV11, AAV12, AAV13.
[C6]A liquid nanoparticle (LNP) comprising the nucleic acid of C1 or the vector of any one of C2-C5.
[C7]A host cell comprising the nucleic acid of C1 or the vector of any one of C2-C5.
[D1]A pharmaceutical composition comprising the polypeptide of any one of A1-A59, the protein of any one of B1-B61, the nucleic acid of C1, the vector of any one of C2-C5, the LNP of C6 or the host cell of C7, and one or more physiologically acceptable carriers, excipients, or diluents.
[E1]A method of treating a disease or disorder associated with C3b in a subject in need thereof, comprising administering to the subject the polypeptide of any one of A1-A59, the protein of any one of B1-B61, the nucleic acid of C1, the vector of any one of C2-C5, the LNP of C6, or the pharmaceutical composition of D1.
[E2] The method of E1, wherein the disease or disorder is selected from the group consisting of dense deposit disease, C3 glomerulonephritis, and age-related macular degeneration.
[E3]A method of modulating alternative complement activity in a subject comprising administering to the subject the polypeptide of any one of A1-A59, the protein of any one of B1-B61, the nucleic acid of C1, the vector of any one of C2-C5, the LNP of C6, or the pharmaceutical composition of D1.
[E4]A method of specifically modulating alternative complement activity in a subject comprising administering to the subject the polypeptide of any one of A1-A59, the protein of any one of B1-B61, the nucleic acid of C1, the vector of any one of C2-C5, the LNP of C6, or the pharmaceutical composition of D1.
[E5]A method of preventing the association of human factor B with human C3b in a subject comprising administering to the subject the polypeptide of any one of A1-A59, the protein of any one of B1-B61, the nucleic acid of C1, the vector of any one of C2-C5, the LNP of C6, or the pharmaceutical composition of D1.
[E6]A method of preventing the association of human factor B with human C3(H₂O) in a subject comprising administering to the subject the polypeptide of any one of A1-A59, the protein of any one of B1-B61, the nucleic acid of C1, the vector of any one of C2-C5, the LNP of C6, or the pharmaceutical composition of D1.
[E7]A method of hindering formation of human C3 convertase in a subject comprising administering to the subject the polypeptide of any one of A1-A59, the protein of any one of B1-B61, the nucleic acid of C1, the vector of any one of C2-C5, the LNP of C6, or the pharmaceutical composition of D1.
[E8]A method of recruiting human I to human C3b in a subject comprising administering to the subject the polypeptide of any one of A1-A59, the protein of any one of B1-B61, the nucleic acid of C1, the vector of any one of C2-C5, the LNP of C6, or the pharmaceutical composition of D1.
[E9]A method of recruiting human I to human C3(H₂O) in a subject comprising administering to the subject the polypeptide of any one of A1-A59, the protein of any one of B1-B61, the nucleic acid of C1, the vector of any one of C2-C5, the LNP of C6, or the pharmaceutical composition of D1.
[E10]A method of facilitating proteolytic cleavage of human C3b in a subject comprising administering to the subject the polypeptide of any one of A1-A59, the protein of any one of B1-B61, the nucleic acid of C1, the vector of any one of C2-C5, the LNP of C6, or the pharmaceutical composition of D1.
[E11]A method of facilitating proteolytic cleavage of human C3(H₂O) in a subject comprising administering to the subject the polypeptide of any one of A1-A59, the protein of any one of B1-B61, the nucleic acid of C1, the vector of any one of C2-C5, the LNP of C6, or the pharmaceutical composition of D1.
[E12]A method of treating an autoimmune disease in a subject comprising administering to the subject the polypeptide or protein of any one of A1 to B70.

### EXAMPLES

The following examples serve only to illustrate the invention and are not intended to limit the scope of the invention.

### Example 1: Binding Affinity and Binding Kinetics of C3b Inhibitor Polypeptides

In this study, C3b inhibitor polypeptides of the present invention were prepared and assessed for their binding affinity and kinetics of binding to C3b and C3(H2O). The polypeptides of the present invention comprise a variable region capable of binding C3b and C3(H2O). In some embodiments, the polypeptides further comprise a half-life extension domain. Exemplary C3b inhibitor polypeptides are shown in Table 1.

TABLE 1
Exemplary C3b inhibitor polypeptides

| Construct | Format | VHH | IgG | Fc mutations | MCP |
|---|---|---|---|---|---|
| AP1 | Fc-VHH-MCP | SEQ ID NO: | IgG4 | F234V, L235E | Domains |
| (SEQ ID NO: 4) | | 10 | | D265G, and S228P | 2-4 |
| AP2 | Fc-VHH-MCP | SEQ ID NO: | IgG4 | T307Q, Q311V, | Domains |
| (SEQ ID NO: 3) | | 10 | | A378V, F234V, | 2-4 |
| | | | | L235E, D265G, and | |
| | | | | S228P | |
| AP3 | VHH-MCP | SEQ ID NO: | — | — | Domains |
| (SEQ ID NO: 5) | | 10 | | | 2-4 |
| AP4 | Fc-VHH-MCP | SEQ ID NO: | IgG1 | T307Q, Q311V, | Domains |
| (SEQ ID NO: 6) | | 10 | | A378V, F234V, | 2-4 |
| | | | | L235E, and D265G | |
| AP5 | Fc-VHH-MCP | SEQ ID NO: | IgG4 | T307Q, Q311V, | Domains |
| (SEQ ID NO: 7) | | 10 | | A378V, F234V, | 2-4; |
| | | | | L235E, D265G, and | S177R |
| | | | | S228P | |
| AP6 | VHH-MCP | SEQ ID NO: | — | — | Domains |
| (SEQ ID NO: 8) | | 13 | | | 2-4 |
| AP7 | VHH-MCP-Fc | SEQ ID NO: | IgG4 | T307Q, Q311V | Domains |
| (SEQ ID NO: 43) | | 13 | | A378V, F234V, | 2-4 |
| | | | | L235E, D265G, and | |
| | | | | S228P | |
| AP8 | VHH-MCP-Fc | SEQ ID NO: | IgG4 | S228P | Domains |
| (SEQ ID NO: 44) | | 13 | | | 2-4 |
| AP9 | Fc-VHH-MCP | SEQ ID NO: | IgG4 | S228P | Domains |
| (SEQ ID NO: 49) | | 13 | | | 2-4 |
| AP10 | VHH-MCP-Fc | SEQ ID NO: | IgG4 | T307Q, Q311V, | Domains |
| (SEQ ID NO: 45) | | 13 | | A378V, F234V, | 2-4 (10 |
| | | | | L235E, D265G, and | amino |
| | | | | S228P | acids) |
| AP11 | VHH-MCP-Fc | SEQ ID NO: | IgG4 | T307Q, Q311V, | Domains |
| (SEQ ID NO: 46) | | 13 | | A378V, F234V, | 2-4 (5 |
| | | | | L235E, D265G, and | amino |
| | | | | S228P | acids) |
| AP12 | Fc-VHH-MCP | SEQ ID NO: | IgG4 | F234V, L235E | Domains |
| (SEQ ID NO: 47) | | 13 | | D265G, and S228P | 2-4 |
| AP13 | Fc-VHH-MCP | SEQ ID NO: | IgG4 | T307Q, Q311V, | Domains |
| (SEQ ID NO: 48) | | 13 | | A378V, F234V, | 2-4 |
| | | | | L235E, D265G, and | |
| | | | | S228P | |
| AP14 | Fc-VHH-MCP | SEQ ID NO: | IgG4 | S228P | Domain |
| (SEQ ID NO: 50) | | 10 | | | 2-4 |
| AP15 | VHH-MCP-Fc | SEQ ID NO: | IgG4 | T307Q, Q311V, | Domain |
| (SEQ ID NO: 51) | | 10 | | A378V, F234V, | 2-4 |
| | | | | L235E, D265G, and | |
| | | | | S228P | |
| AP16 | Fc-VHH-MCP | SEQ ID NO: | IgG4 | F234V, L235E | Domains |
| (SEQ ID NO: 52) | | 10 | | D265G, and S228P | 2-4 |
| AP17 | Fc-VHH-MCP | SEQ ID NO: | IgG4 | F234V, L235E | Domains |
| (SEQ ID NO: 53) | | 55 | | D265G, and S228P | 2-4 |
| AP18 | Fc-VHH-MCP | SEQ ID NO: | IgG4 | F234V, L235E | Domains |
| (SEQ ID NO: 54) | | 13 | | D265G, and S228P | 2-4 |
| Ctrl 1 | VHH-Fc | SEQ ID NO: | IgG4 | T307Q, Q311V, | — |
| (SEQ ID NO: 2) | | 13 | | A378V, F234V, | |
| | | | | L235E, D265G, and | |
| | | | | S228P | |
| Ctrl2 | VHH-Fc | SEQ ID NO: | IgG4 | S228P | — |
| (SEQ ID NO: 1) | | 10 | | | |
| VHH1 | VHH | SEQ ID NO: | — | — | — |
| | | 13 | | | |
| VHH2 | VHH | SEQ ID NO: | — | — | — |
| | | 10 | | | |
| VHHV0 | VHH | SEQ ID NO: | | | |
| | | 55 | | | |

The binding affinity and kinetics of binding of antibody to C3b and C3(H2O) were measured by surface plasmon resonance using a Biacore8K instrument (Cytiva). Goat Anti-Human IgG, Fc fragment specific antibody (Jackson ImmunoResearch Inc.) was immobilized onto a carboxymethylated dextran CM5 sensor chip (Cytiva) via standard amine coupling protocols. All analyses were conducted at 25° C. The running and dilution buffers used throughout the experiment consisted of HBS-EP+, pH7.4 (Cytiva), supplemented with 0.1 M HEPES, 1.5 M NaCl, 0.03 M EDTA and 0.500 v/v Surfactant P20. The antibody was captured in individual flow cells at a flow rate of 10 μL/min, with the injection time optimized for each antibody to achieve an Rmax between 12 and 25 resonance units (RU). Buffer, C3b, or C3(H₂O) diluted in running buffer was injected sequentially over both a reference surface (no antibody captured) and the active surface (captured antibody) at a flow rate of 40 μL/min for 240 seconds. The dissociation phase was monitored for up to 5400 seconds to accurately capture the slower off-rate kinetics.

Surface regeneration was performed using 30-second injections of 10 mM glycine, pH 1.5, at a flow rate of 30 μL/min. The concentration range of C3b (Complement Technology, Inc) or C3(H₂O) (prepared in house from C3, Complement Technology, Inc.) tested was between 200 nM and 0.195 nM, using a 2-fold serial dilution series. Kinetic parameters, including ka (association rate constant), kd (dissociation rate constant), and KD (equilibrium dissociation constant), were determined using Biacore Insight Evaluation Software (Cytiva) with double reference subtraction to ensure data accuracy. The resulting kinetic parameters for the interaction of antibody with C3b and C3(H₂O) at 25° C. are summarized in the Table 2.

TABLE 2
Kinetic parameters of Exemplary C3b Inhibitor Polypeptides

| Ligand | Analyte | ka (1/Ms) | kd (1/s) | KD (nM) |
|---|---|---|---|---|
| AP2 | hu C3b | 3.43E+05 | 5.31E−06 | 0.015 |
| | cyno C3b | 1.68E+05 | 2.08E−03 | 10.04 |
| | mu C3b | 2.75E+05 | 1.85E−04 | 0.67 |
| | rat C3b | 1.28E+05 | 1.95E−04 | 1.52 |
| | hu C3H2O | 4.03E+05 | 2.78E−06 | 0.007 |
| | cyno | 5.09E+05 | 8.80E−04 | 1.53 |
| | C3H2O | | | |
| | mu C3H2O | 4.75E+04 | 2.01E−04 | 4.24 |
| | rat C3H2O | 1.37E+05 | 1.45E−04 | 1.06 |
| AP1 | hu C3b | 3.33E+05 | 5.34E−06 | 0.016 |
| | cyno C3b | 1.97E+05 | 2.07E−03 | 8.96 |
| | mu C3b | 3.04E+05 | 1.76E−04 | 0.57 |
| | rat C3b | 1.50E+05 | 1.89E−04 | 1.27 |
| | hu C3H2O | 4.27E+05 | 4.50E−06 | 0.011 |
| | cyno | 6.18E+05 | 9.70E−04 | 1.3 |
| | C3H2O | | | |
| | mu C3H2O | 5.09E+04 | 2.10E−04 | 4.05 |
| | rat C3H2O | 1.41E+05 | 1.47E−04 | 1.04 |

### Example 2: Blocking of Factor B Binding to C3b by C3b Inhibitor Polypeptides

This example shows that C3b inhibitor polypeptides of the present invention effectively block factor B binding to C3b and irreversibly inhibit alternative pathway.

In the complement system, factor B directly binds to C3b, forming a complex that is crucial for activating the alternative pathway of complement cascade; essentially, C3b acts as a platform for factor B to be cleaved by factor D, generating the active enzyme “C3bBb” which is a key component of the C3 convertase, responsible for cleaving and activating more C3 molecules.

### Factor B Blocking ELISA Assay

Purified C3b (Complement Technology, Inc.) was used to coat ELISA plates at a concentration of 20 g/mL in phosphate-buffered saline (PBS), pH 7.4. Plates were incubated overnight at 4° C. to ensure proper coating. After incubation, plates were washed three times with Wash Buffer, which consisted of 10 mM HEPES, 50 mM NaCl, 2 mM MgCl₂, 0.05% Tween-20, and 2.5 mM NiCl₂. The plates were blocked with 5% Blotto (Santa Cruz Biotechnology) prepared in Wash Buffer for 1 hour at room temperature (RT) to minimize non-specific binding. Following blocking, the plates were washed again using the Wash Buffer. Serial dilutions of test articles (TAs) were prepared in Wash Buffer supplemented with 5% Blotto. These dilutions were added to the wells of the ELISA plates and incubated for 1 hour at RT to allow for potential interactions. After washing the plates three times with Wash Buffer, 100 nM of in-house biotinylated factor B (fB) (Complement Technology, Inc.) was added to the wells, which are prepared in Wash Buffer with 5% Blotto. The plates were then incubated for 1 hour at RT to enable binding interactions. After incubation, the plates were washed to remove unbound materials. Streptavidin-HRP (AbCam), appropriately diluted in Wash Buffer with 5% Blotto, was added to the wells. Plates were incubated for 1 hour at RT. For signal development, TMB substrate was added, and the reaction was stopped by the addition of 1 N H₂SO₄. Absorbance at 450 nm (A450) was measured using a SpectraMAX M3 Plate Reader. fB binding curves were generated, and the interaction and relative binding were analyzed using GraphPad Prism software. Results were interpreted to determine the blocking characteristics of the test articles.

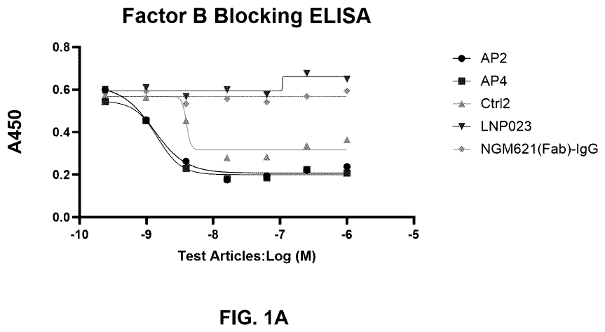

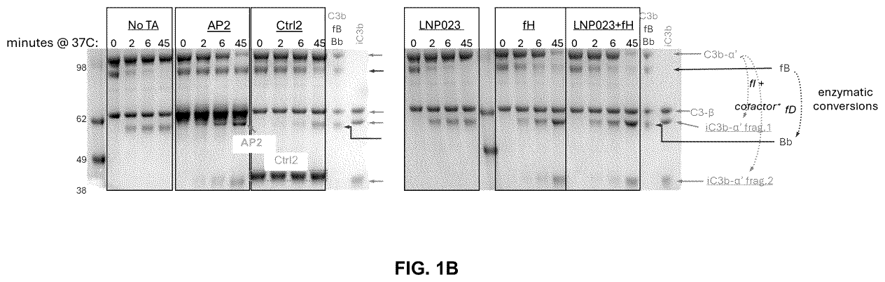

FIG. 1A shows that exemplary C3b inhibitor polypeptides (AP2 and AP4) effectively block factor B from binding to C3b. LNPO23 (MedChem Express) is a small molecule factor B inhibitor,

### Protein-Based AP Reaction as Determined by SDS-PAGE

The mechanism of action of alternative pathway (AP) inhibition was evaluated in vitro using purified protein components. Reactions were conducted in GVB0/MgEGTA buffer (Complement Technology, Inc.) supplemented with the following components: C3b (1.0 μM), factor B (0.54 μM), factor D (20 nM), factor I (7.5 nM) (all sourced from Complement Technology, Inc.), and one of the test article inhibitors (1.0 μM) or factor H (1.5 μM) (Complement Technology, Inc.). The reactions were incubated at room temperature, and samples were collected at predetermined time points of 0, 2, 6, and 45 minutes. The reaction products were analyzed via reducing SDS-PAGE and visualized by staining with Coomassie blue to assess the extent and nature of protein interactions and processing.

In alternative pathway of complement, factor B (fb) is converted to Bb and C3b is converted to iC3b (inactivated C3b). FIG. 1B shows that in presence of AP2, there is no conversion of factor B (fb) to Bb, and very slow conversion of C3b to iC3b.

### Example 3: Cryo-EM Structure of C3b Inhibitor Polypeptide in Complex with C3b

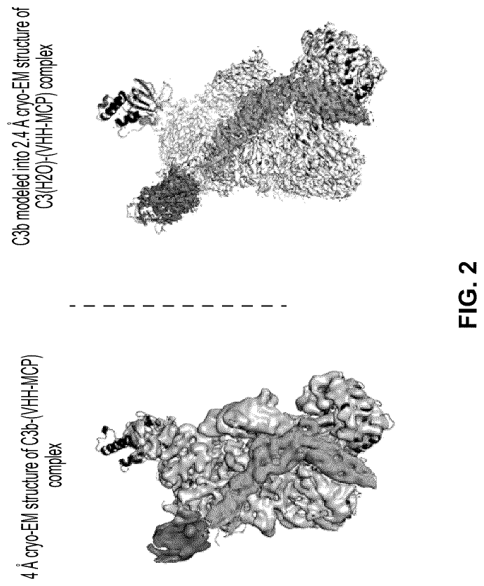

In this example, high resolution of cryo-EM structures of C3b or C3 in complex with AP6 were determined. FIG. 2 shows the detailed view of the interactions between the anti-C3b VHH linked to MCP in combination with C3b/C3(H2O).

### Example 4: C3b Inhibitor Polypeptides Inhibits Alternative Pathway (AP) Mediated Activities

This example shows that the exemplary C3b inhibitor polypeptides of the present invention effectively inhibit C3b deposition, MAC deposition, C3 consumption, and hemolysis, which are indications of alternative pathway (AP) AP-C3 convertase activities.

### C3b Deposition Assay

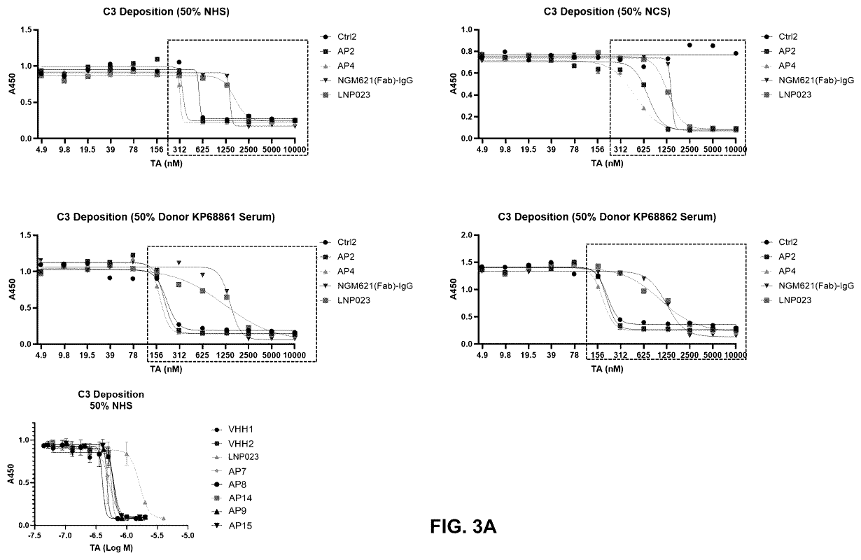

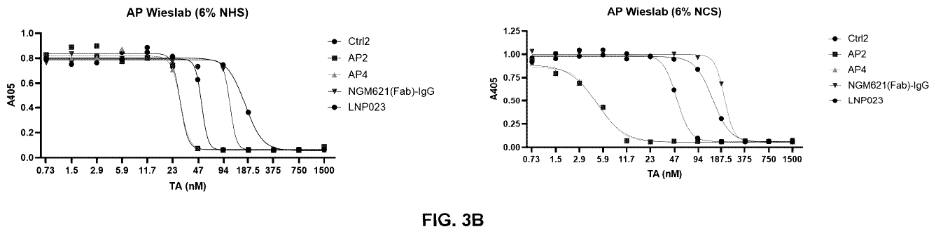

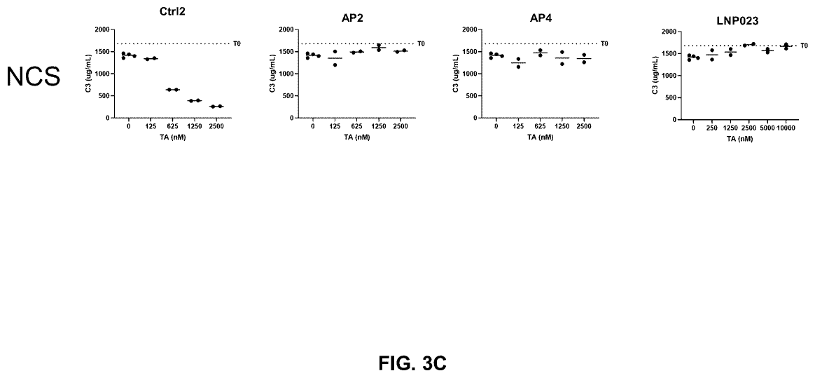

Pre-activated Zymosan (Complement Technology, Inc.) was coated onto ELISA plates at a concentration of 0.2 mg/mL in PBS, pH 7.4, and incubated overnight at 4° C. Following incubation, the plates were washed three times with Wash Buffer (PBS with 0.05% Tween-20) and subsequently blocked with 5% Blotto in Wash Buffer (Santa Cruz Biotechnology) for 1 hour at 37° C. to minimize non-specific binding. Serial dilutions of test articles (TAs) were prepared in assay buffer (GVBo, 10 mM MgEGTA—Complement Technology, Inc.) and spiked into a final concentration of 50% normal human serum (NHS), normal cynomolgus monkey serum (NCS) (Complement Technology, Inc), or individual human donor serum (Research Blood Components, LLC). The TA/50% serum mixtures were pre-incubated for 30 minutes on ice. After blocking, the ELISA plates were washed, and diluted test articles in 50% serum were added to the wells. Plates were incubated for 1 hour at 37° C. on a shaker plate at 150 rpm. Following the incubation, plates were washed, and bound C3 derivatives were detected using 7C12, a non-inhibitory anti-C3/C3b mouse IgG (Cedarlane USA), diluted to 5 g/mL in blocking buffer (Wash Buffer with 500 Blotto). After a 1-hour incubation at 37° C. on a shaker plate at 150 rpm, the plates were washed and developed using a suitable HRP-conjugated secondary antibody (Jackson Immunoresearch Laboratories), followed by the addition of TMB substrate. The reaction was stopped with 0.1 N H₂SO₄, and absorbance at 450 nm (A450) was measured using a SpectraMAX M3 Plate Reader. Binding curves were plotted as shown in FIG. 3A, and data were analyzed using GraphPad Prism software to determine the IC₅₀ and IC90 values of the test articles. IC50s and IC90s of each sample are quantified in Table 3. IC50s of additional exemplary C3b inhibitor polypeptides are similarly shown in Table 4.

TABLE 3
IC50 and IC90 Values of C3 Deposition
IC50 (nM)

| | | | Donor Serum | |
|---|---|---|---|---|
| TAs | NHS | NCS | KP68861 | KP68862 |
| Ctrl2 | 563.5 | | 214.4 | 211 |
| AP2 | 347.2 | 714.2 | 197.2 | 207.6 |
| AP4 | 318.7 | 492.3 | 175.8 | 184.7 |
| NGM621(Fab)-Ig | 1428 | 1446 | 1441 | 1247 |
| LNP023 | 1654 | 1302 | 1228 | 1040 |

IC90 (nM)

| | | | Donor Serum | |
|---|---|---|---|---|
| TAs | NHS | NCS | KP68861 | KP68862 |
| Ctrl2 | 603.3 | | 312.4 | 304.1 |
| AP2 | 396.2 | 1132 | 270.5 | 285.4 |
| AP4 | 367.2 | 1081 | 237.2 | 266.7 |
| NGM621(Fab)-Ig | 1499 | 1622 | 2043 | 2276 |
| LNP023 | 2487 | 2062 | 6252 | 3469 |

TABLE 4
IC50 Values of C3 Deposition with Additional
Exemplary C3b Inhibitor Polypeptides
IC50 (μM)

| TAs | NHS |
|---|---|
| VHH1 | .62 |
| VHH2 | .62 |
| LNP023 | 1.63 |
| AP7 | .52 |
| AP8 | .40 |
| AP14 | .56 |
| AP9 | .48 |
| AP15 | .59 |

The data indicate that C3b inhibitor polypeptides of the present invention (e.g., AP2, AP4 AP7, and AP8) perform better than all the other controls in all four serum conditions. IC50 values are lower in donor serum as there is less debris present in the serum.

### AP Wieslab Assay

Test Articles (TAs) were evaluated for alternative pathway (AP) inhibition using 6% normal human serum (NHS) and 6% normal cynomolgus monkey serum (NCS). Serial dilutions of TAs were preincubated with 6% serum for 30 minutes on ice. Following preincubation, 50 μL of the serum-TA mixtures were added to pre-coated, ready-to-use microplate wells supplied in the kit. Samples were incubated for 1 hour at 37° C. with shaking. The wells were then washed three times with the kit-provided Wash Buffer. Subsequently, 50 μL of the conjugate solution was added to each well, and the plate was incubated for 30 minutes at room temperature (RT). The wells were washed three times again as before. Next, 50 μL of the substrate solution was added to each well, and the plate was incubated for 30 minutes at RT. Finally, the absorbance was measured at 405 nm using SpectraMAX M3 Plate Reader.

Data in FIG. 3B and IC50 and IC90 values shown in Table 5 and Table 6 illustrate that C3b inhibitor polypeptides of the present invention (e.g., AP2 and AP4) have the greatest inhibition of MAC deposition in both NHS and NCS in this assay.

TABLE 5
MAC deposition IC50 and IC90 of Exemplary
C3b Inhibitor Polypeptides
IC50 (nM)

| TAs | NHS | NCS |
|---|---|---|
| Ctrl2 | 52.12 | 50.69 |
| AP2 | 28.88 | 4.887 |
| AP4 | 29.09 | 5.288 |
| NGM621(Fab)-IgG | 113.2 | 206 |
| LNP023 | 171.2 | 145.6 |

IC90 (nM)

| TAs | NHS | NCS |
|---|---|---|
| Ctrl2 | 63.02 | 78.01 |
| AP2 | 36.38 | 11.76 |
| AP4 | 38.06 | 11.16 |
| NGM621(Fab)-IgG | 137.3 | 278.1 |
| LNP023 | 289.3 | 254.4 |

TABLE 6
MAC deposition IC50 of Additional Exemplary
C3b Inhibitor Polypeptides

| TAs | IC50 (nM) |
|---|---|
| VHH1 | 20 |
| VHH1-MCP | 39 |
| AP8 | 22 |
| AP9 | 22 |
| LNP023 | 18 |

### C3 Consumption Assay

Test articles (TAs) were prepared at 10× the desired concentrations in PBS, pH 7.4 and spiked into normal cynomolgus monkey serum (NCS) (Complement Technology, Inc.). The mixtures were aliquoted into 96-well PCR plates to achieve a final 1×TA concentration in 90% serum. The plates were sealed with foil, incubated at 37° C. for 6 hours, and subsequently frozen at −80° C. Control samples consisting of 90% serum and PBS (without TAs) were prepared and immediately frozen at −80° C. High-binding ELISA plates were coated overnight at 4° C. with the C3-specific antibody at a concentration of 1 μg/mL in PBS (pH 7.4). The next day, plates were washed three times with PBS containing 0.05% Tween-20 (PBS-T) and blocked with 1% bovine serum albumin (BSA) in PBS (pH 7.4) for 1 hour at 37° C. Following the blocking step, the plates were washed again with PBS-T. During the blocking process, the 1×TA/90% serum samples were thawed and maintained on ice. These samples were diluted 1:100 in a solution containing 1% BSA and 10 mM EDTA, followed by further dilution in blocking buffer to a final dilution of 1:100,000. Twelve-point standard curves of cynomolgus monkey C3 (Complement Technology, Inc.) were prepared in blocking buffer, starting at a concentration of 1 μg/mL. Diluted samples and standards (50 μL) were added to the ELISA plates and incubated for 1 hour at room temperature. Plates were then washed thoroughly with PBS-T, and C3 detection was performed using the biotinylated non-overlapping C3 antibody at a concentration of 1 μg/mL in blocking buffer. Streptavidin-HRP (Abcam) was subsequently added, and colorimetric detection was achieved using the TMB substrate. The enzymatic reaction was stopped with 0.1 N sulfuric acid (H₂SO₄), and absorbance at 450 nm (A450) was measured using a SpectraMAX M3 Plate Reader. C3 concentrations in the samples were quantified by back-calculation using standard curves generated from the known concentrations of cynomolgus monkey C3. Data analysis was performed using the SpectraMAX software.

Data in FIG. 3C illustrates that C3b inhibitor polypeptides of the present invention (e.g., AP2 and AP4) do not cause C3 consumption in cynomolgus monkey serum.

### Example 5: C3b Inhibitor Polypeptides do not Interfere with Classical Pathway C3 Convertase Activity, but Affect Downstream Components Shared by Both the Alternative Pathway and Classic Pathway

This example shows that the C3b inhibitor polypeptides of the present invention do not inhibit the classical pathway at the C3 convertase step This example shows that under certain conditions C3b inhibitor polypeptides have no noticeable effect on the classical pathway of the C3 convertase step, while other polypeptides show classical pathway inhibition at levels similar to that of alternative pathway inhibition when analyzed for shared downstream components. These tests were performed by measuring the deposit of the C3b on the surface of classical pathway Wieslab assay plates, with similar analysis for comparison to alternative pathway inhibition as described in Example 4.

Classical Pathway Assay with 50% Factor-B Depleted Serum

Test Articles (TAs) were evaluated for classical pathway (CP) inhibition using 50% Factor B-Depleted serum (Complement Technology, Inc.). The Wieslab CP kit (SVAR Life Science) was used with several modifications. Serial dilutions of TAs were preincubated with 50% Factor B-Depleted serum for 30 minutes on ice. Following preincubation, 100 μL of the serum-TA mixtures were added to pre-coated, ready-to-use microplate wells supplied in the kit. Samples were incubated for 1 hour at 37° C. with shaking. Following the incubation, plates were washed and bound C3 derivatives were detected using 7C12 (a non-inhibitory anti-C3/C3b mouse IgG (Cedarlane, USA), diluted to 5 μg/mL in PBS, pH 7.4. The plates were washed and detected using F(ab′)2 Fragment Rabbit Anti-Mouse IgG (H+L) secondary antibody (Jackson Immunoresearch Laboratories), followed by the addition of TMB substrate. The reaction was stopped with 0.1 N H2SO4, and absorbance at 450 nm (A450) was measured using a SpectraMAX M3 Plate Reader.

### Classical Pathway Assay

Test Articles (TAs) were evaluated for classical pathway (CP) inhibition using Wieslab CP kit (SVAR Life Science). Experiments were performed under specified conditions or with several modifications. Serial dilutions of TAs were preincubated with 3% C5-Depleted serum for 30 minutes on ice. Following preincubation, 100 μL of the serum-TA mixtures were added to pre-coated, ready-to-use microplate wells supplied in the kit. Samples were incubated for 1 hour at 37° C. with shaking. Following the incubation, plates were washed and bound C3 derivatives were detected using 7C12, a non-inhibitory anti-C3/C3b mouse IgG (Cedarlane USA), diluted to 5 μg/mL in PBS, pH 7.4. The plates were washed and detected using F(ab′)2 Fragment Rabbit Anti-Mouse IgG (H+L) secondary antibody (Jackson Immunoresearch Laboratories), followed by the addition of TMB substrate. The reaction was stopped with 0.1 N H2SO4, and absorbance at 450 nm (A450) was measured using a SpectraMAX M3 Plate Reader.

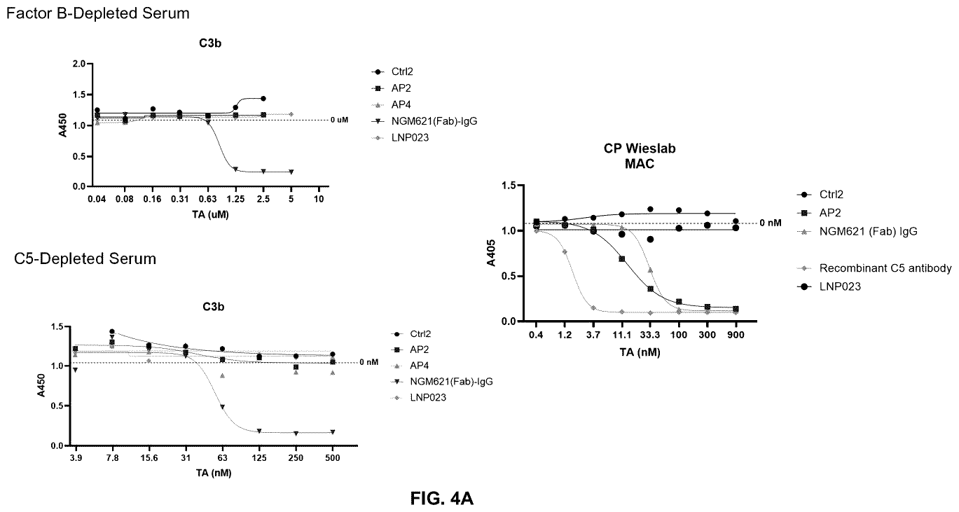

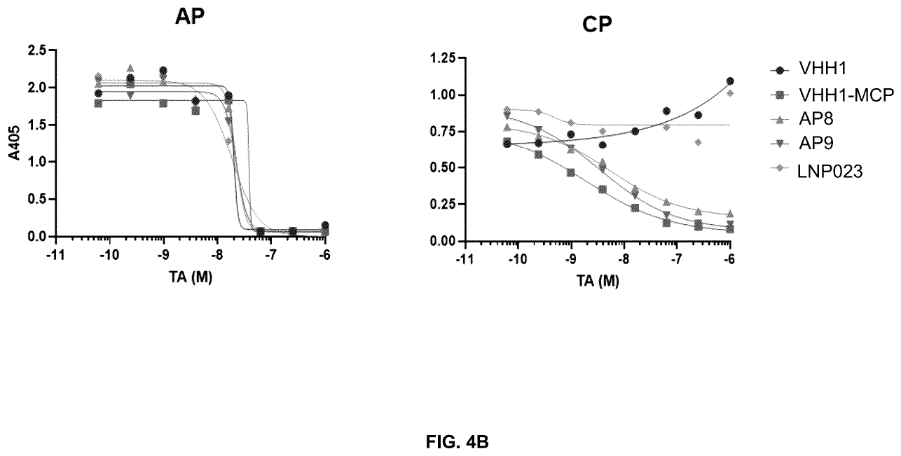

If the CP C3 convertase is formed, C3 will be cleaved and C3b will be deposited on the surface of the plate. Data in FIG. 4A illustrate that select C3b inhibitor polypeptides of the present invention (e.g., AP2 and AP4) do not affect the C3b deposition, indicating that classical pathway C3 convertase is not inhibited by these constructs. On the other hand, the control NGM621(Fab)-IgG inhibits C3b deposition. An additional Wieslab assay was performed to analyze the formation of the membrane attack complex (MAC) following treatment with C3b inhibitor polypeptides, as shown in FIG. 4A. This assay showed the results of C3b inhibition on downstream effectors seen in both the classical pathway and the alternate pathway. Surprisingly, it was found that although the C3b inhibitor polypeptide AP2 did not show an effect on C3b deposition through the classical pathway, MAC formation was still reduced. This shows that C3b inhibitor polypeptides may not directly affect C3b deposition through the classical pathway, but are still capable of producing downstream components shared by both the classical and alternate pathways, as evidenced by the reduction in MAC formation.

Wieslab assays were performed with additional exemplary C3b inhibitor polypeptide constructs and analyzed for C3 content in both the alternative and classical pathways. As determined by IC50 values shown in Table 7, a number of additional C3b inhibitory polypeptides (e.g., AP8 and AP9) showed reductions in C3b as determined by plate absorbance when analyzed for both the alternative and classical pathways. Under the experimental conditions of 6.5% NHS (for the alternative pathway) and 2.0% NHS (for the classical pathway, it was determined that the IC50 for AP8 and AP9 C3b inhibitor polypeptides showed similar inhibition across pathways when A450 was quantified.

Additional Wieslab assays were performed for the same C3b inhibitor polypeptides to quantify A405 in across both alternative and classical pathways, as shown in Table 8 and FIG. 4B. Similarly to A405, quantification of C3 cleavage showed that AP8 and AP9 affected both alternative and classical pathways. For AP8, an IC50 of 22 nM and 5 nM was quantified in the alternative and classical pathways, respectively. For AP9, an IC50 of 22 nM and 3 nM was quantified in the alternative and classical pathways, respectively. Interestingly, the dynamic profile of the Wieslab assay differed between alternative and classical pathways. Quantification of the alternative pathway showed a rapid inhibition. from inactive to full cleavage, while the classical pathway showed a gradual inhibition across increasing dosing.

TABLE 7
Alternative and classical pathway IC50 values in exemplary
C3b inhibitor polypeptides (M)

| | Alternative | Classical |
|---|---|---|
| TAs | IC50 (M) | |
| VHH1 | 8.7E−08 | |
| VHH1-MCP | 2.1E−08 | 1.2E−08 |
| MCP | 2.4E−07 | 4.2E−07 |
| AP16 | 2.3E−08 | 5.6E−09 |
| AP8 | 2.3E−08 | 2.2E−08 |
| AP14 | 2.9E−08 | 3.4E−08 |
| AP9 | 4.4E−08 | 5.1E−08 |

TABLE 8
Alternative and classical pathway IC50 values in exemplary
C3b inhibitor polypeptides (nM)

| | Alternative | Classical |
|---|---|---|
| TAs | IC50 (nM) | |
| VHH1 | 20 | |
| VHH1-MCP | 39 | 2 |
| LNP023 | 18 | |
| AP8 | 22 | 5 |
| AP9 | 22 | 3 |

In conclusion, certain exemplary C3b inhibitory peptides (such as AP2 and AP4) selectively affect the alternative pathway while showing minimal effect of the classical pathway. Other C3b inhibitory polypeptides (such as AP8 and AP9) demonstrated similar inhibition of both the classical and alternative pathways. Taken together, these results indicate that C3b inhibitory polypeptides can be designed in ways to either selectively or generally target C3 convertase activity through either pathway of activation.

### Example 6: C3b Inhibitor Polypeptides Inhibit the Alternate Pathway in Hemolytic Assays of Cyno and Human Sera Samples

This example demonstrates ex vivo effects of C3b inhibitor polypeptides on the alternate pathway in mammalian samples using an assay for hemolytic activity.

The hemolytic assay is designed to assess test antibodies' ability to block the activation of the human complement cascade through the alternative pathway, and thereby inhibit complement mediated lysis of target cells. The assay is initiated with the pre-incubation of test antibody with pooled normal human serum (Complement Technology, NHS) or healthy human donor serum (Research Blood Components, #009) containing all the necessary AP complement components (C3, factor B, and factor D). The pre-incubation mixes are then transferred to wells containing rabbit erythrocytes (Complement Technology, #B302) in an assay buffer containing 21 mM HEPES (Gibco, #15630080), 0.1% gelatin (Sigma Aldrich, #G9382), 144 mM NaCl (Sigma Aldrich, #S3014), 10 mM MgCl₂ (Sigma Aldrich, #M1028), and 10 mM EGTA (BioWORLD, #40520008-3). Activation of both the classical and lectin pathways is blocked by the presence of EGTA due to both the classical and lectin pathways being Ca²⁺-dependent. The addition of MgCl₂ is necessary to facilitate the activity of the Mg²⁺-dependent AP. After an incubation period of 1 hour at 37° C. with rotation at 1000 rpm the extent of hemolysis is assessed as a function of released hemoglobin levels (A414). Serum from both cynomolgus monkeys and humans were treated with either a C3b inhibitor polypeptide or control and analyzed for A414 or percent hemolysis.

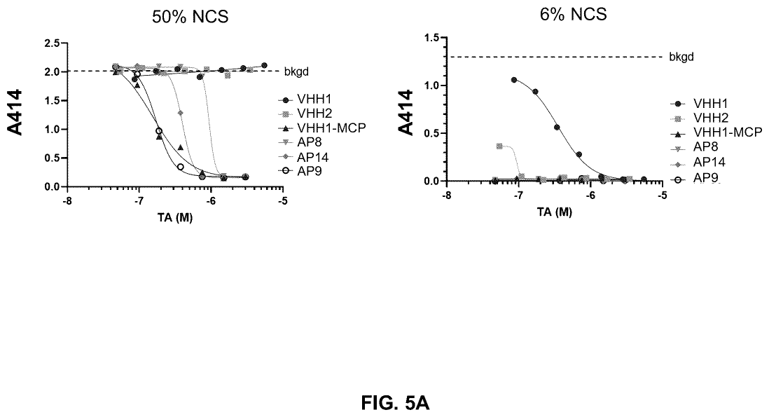

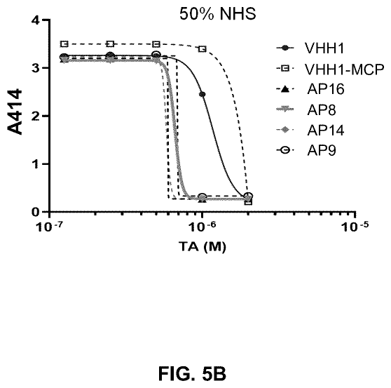

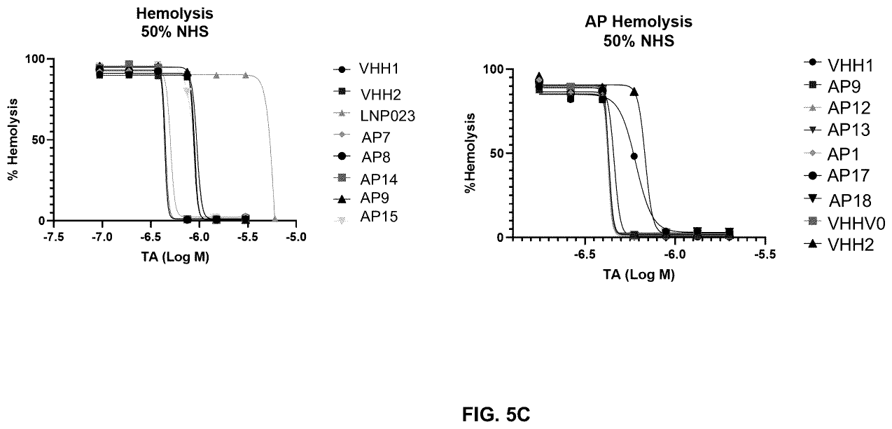

Data were collected from both 50% and 6% NCS from cynomolgus monkeys. As shown in FIG. 5A and Table 9, treatment with a C3b inhibitory polypeptide dramatically inhibited the alternative pathway relative to controls, with IC50 values over 100 times (2 orders of magnitude) lower for C3b inhibitor polypeptides relative to VHH controls in 50% NCS conditions and over 10 times (1 order of magnitude) lower in 6% NCS conditions.

Similar results were observed in human sera analyzed at 50% NHS, as seen in FIG. 5B and Table 9. Increased inhibition of the alternative pathway was observed at a level about twice as efficient relative to the VHH control across all C3b inhibitor polypeptides tests based on IC50 values.

TABLE 9
IC50 for hemolytic assay in cynomolgus and
human serum samples

| | IC50 (M) | | |
|---|---|---|---|
| | Cynomolgus | | Human |
| TAs | 50% NCS | 6% NCS | 50% NHS |
| VHH1 | >1.0E−5 | 3.5E−7 | 1.2E−6 |
| VHH2 | >1.0E−5 | <1.0E−7 | ND |
| VHH1-MCP | 1.5E−7 | <1.0E−8 | 3.5E−6 |
| AP8 | 9.4E−7 | <1.0E−8 | 6.6E−7 |
| AP14 | 4.0E−7 | <1.0E−8 | 6.0E−7 |
| AP9 | 1.7E−7 | <1.0E−8 | 5.0E−7 |

Additionally, human sera samples were analyzed for the efficiency of hemolysis when treated with C3b inhibitory polypeptides. Various constructs were analyzed for both general effects on hemolysis (FIG. 5C and Table 10) as well as alternative pathway-specific hemolysis (FIG. 5C and Table 11). It was observed that using 50% NHS, C3b inhibitory polypeptides effectively reduced both general and alternative pathway-specific hemolysis, with IC50 values for all constructs tested (including AP1, AP9, AP12, and AP13) lower than VHH alone. The degree of effect was more pronounced in general hemolysis, though reduction were seen for both parameters tested.

TABLE 10
IC50 values of C3b inhibitory polypeptides analyzed for general hemolysis

| | IC50 NHS |
|---|---|
| TAs | (μM) |
| VHH1 | 0.95 |
| VHH2 | 0.89 |
| LNP023 | 5.99 |
| AP7 | 0.51 |
| AP8 | 0.44 |
| AP14 | 0.45 |
| AP9 | 0.88 |
| AP15 | 0.89 |

TABLE 11
IC50 values of C3b inhibitory polypeptides analyzed for
alternative pathway-specific hemolysis

| | IC50 NHS |
|---|---|
| TAs | (nM) |
| VHH1 | 605.3 |
| AP9 | 429.4 |
| AP12 | 428 |
| AP13 | 430.6 |
| AP1 | 423.4 |
| AP17 | 459.4 |
| AP18 | 426.7 |
| L144V0 | 425.5 |
| VHH2 | 683.9 |

In conclusion, this example demonstrates that C3b inhibitor polypeptides can be used to effectively inhibit the alternative pathway and hemolysis in mammalian samples, demonstrating the effectiveness of multiple constructs.

### Example 7: C3b Inhibitor Polypeptides Reduce Glomerular C3b Deposits in a Mice Model with a Hyperactive Alternative Pathway

This example shows that the treatment of a C3-related disorder mouse model with a C3b-inhibitor polypeptide can reduce C3b deposits in vivo. Specifically, Treatment of Cfh −/− mice with AP2 significantly reduced pathologies associated with a hyperactive alternative pathway.

In order to assess the effects of C3b inhibitor polypeptides in mouse models that simulate C3-related disorders, Cfh−/− mice were used for in vivo experiments. These mice lack complement factor H, the primary negative regulator of the alternative pathway. This leads to a hyperactive alternative pathway, with chronic activation that drives continuous C3 consumption that significantly depletes plasma C3 and results in C3 deposition along the glomerular basement membrane, causing C3-dominant glomerulopathy in mice.

In order to assess the effects of C3b inhibitor polypeptides, mice were treated with either MVZ (an isotype control), AP2, or vehicle with two different administration routes (intraperitoneal or intravascular) for 7 or 4 days, respectively, as shown in Table 12.

TABLE 12
Experimental Conditions and Groups for Treatment of
Hyperactive Alternative Pathway by C3b inhibitors

| | | Dose and Frequency | Kidney Collection |
|---|---|---|---|
| Group | Treatment | (mg/kg; days) | Day |
| 1 (n = 4) | MVZ IP | 50; 7 | 7 |
| 2 (n = 4) | MVZ IV | 50; 4 | 4 |
| 3 (n = 4) | AP2 IP | 50; 7 | 7 |
| 4 (n = 4) | AP2 IV | 50; 4 | 4 |
| 5 (n = 3) | None | | 7 |
| 6 (n = 3) | None | | 4 |

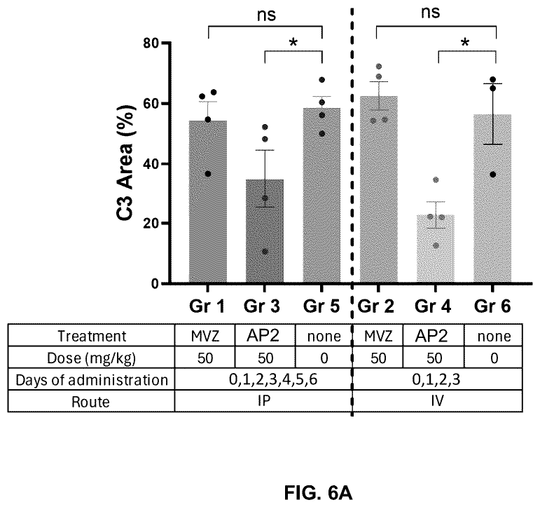

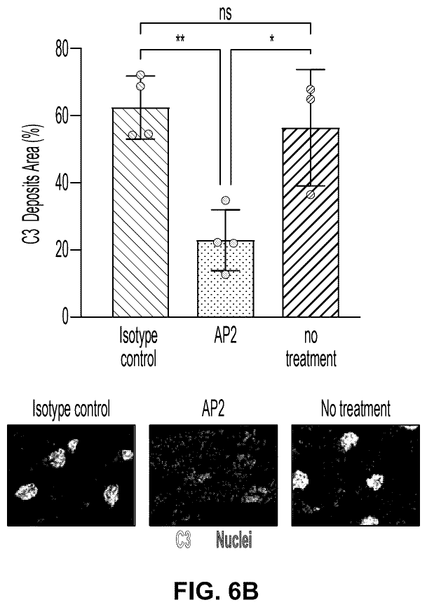

Treatment with AP2, administered either intraperitoneal (I.P.) for 7 days or intravascular (I.V.) for 4 days, reduced C3 deposits detected by immunofluorescence compared with both control groups, shown as mean±SEM (FIG. 6A). In the IV treatment group, quantification of the C3 positive area by immunofluorescence, together with the representative image of C3 deposits shown in FIG. 6B (mean±SD), demonstrated a statistically significant reduction in C3 deposition relative to both untreated mice and mice receiving an isotype control (one way ANOVA).

These results indicate that C3b inhibitory polypeptides can be used to effectively treat C3-related diseases or disorders, and that a hyperactive alternative pathway and related pathologies can be mediated by inhibition of C3b. Furthermore, these results indicate an effectiveness of AP2 when used in vivo.

1. A polypeptide comprising
a VHH that binds to C3b comprising a HCDR1 comprising NYHMG (SEQ ID NO: 16), a HCDR2 comprising TIIRTGETIYYADSVKG (SEQ ID NO:17). and a HCDR3 comprising ATSGWNIKTVTPYEY (SEQ ID NO:18), and
an amino acid sequence of Membrane Cofactor Protein (MCP) comprising SEQ ID NO: 9.

2. The polypeptide of claim 1 wherein the polypeptide comprises from the N-terminus to the C-terminus, the VHH and the amino acid sequence of MCP.

3. The polypeptide of claim 1 further comprising a Fc domain comprising amino acid substitutions of S228P, F234V, L235E, D265G, T307Q, Q311V and A378V, according to EU index.

4. The polypeptide of claim 3 comprising from the N-terminus to the C-terminus, the Fc domain, the VHH and the amino acid sequence of MCP.

5. The polypeptide of claim 1, wherein the VHH comprises an amino acid sequence of SEQ ID NO: 10.

6. The polypeptide of claim 3, wherein the Fc domain is a human IgG4 Fc domain.

7. The polypeptide of claim 3, wherein the Fc domain comprises the amino acid sequence of SEQ ID NO: 33.

8. The polypeptide of claim 4, wherein the polypeptide comprises the Fc domain of SEQ ID NO: 33, the VHH of SEQ ID NO: 10 and the amino acid sequence of MCP of SEQ ID NO: 9.

9. The polypeptide of claim 4, wherein the Fc domain is linked to the VHH via a first peptide linker and the VHH is linked to the sequence of MCP via a second peptide linker.

10. The polypeptide of claim 9, wherein the first peptide linker comprises GGGGGS (SEQ ID NO: 41) and the second peptide linker comprises GGGSGGGSGGGS (SEQ ID NO: 39).

11. A protein comprising two identical polypeptides, each polypeptide comprising,
a VHH that binds to C3b comprising a HCDR1 comprising NYHMG (SEQ ID NO: 16), a HCDR2 comprising TIIRTGETIYYADSVKG (SEQ ID NO:17). and a HCDR3 comprising ATSGWNIKTVTPYEY (SEQ ID NO:18), and
an amino acid sequence of Membrane Cofactor Protein (MCP) comprising SEQ ID NO: 9.

12. The protein of claim 11, wherein each polypeptide comprises from the N-terminus to the C-terminus, the VHH and the amino acid sequence of MCP.

13. The protein of claim 11, wherein each polypeptide further comprises a Fc domain comprising amino acid substitutions of S228P, F234V, L235E, D265G, T307Q, Q311V and A378V, according to EU index

14. The protein of claim 13, wherein each polypeptide comprises from the N-terminus to the C-terminus, the Fc domain, the VHH and the amino acid sequence of MCP.

15. The protein of claim 11, wherein the VHH comprises an amino acid sequence of SEQ ID NO: 10.

16. The protein of claim 13, wherein the Fc domain is a human IgG4 Fc domain.

17. The protein of claim 13, wherein the Fc domain comprises the amino acid sequence of SEQ ID NO: 33.

18. The protein of claim 14, wherein the Fc domain is linked to the VHH via a first peptide linker and the VHH is linked to the sequence of MCP via a second peptide linker.

19. The protein of claim 18, wherein the first peptide linker comprises GGGGGS (SEQ ID NO: 41) and the second peptide linker comprises GGGSGGGSGGGS (SEQ ID NO: 39).

20. The protein of claim 14, wherein each polypeptide comprises the Fc domain of SEQ ID NO: 33, the VHH domain of SEQ ID NO: 10 and the amino acid sequence of MCP of SEQ ID NO: 9.

21. A protein comprising two identical sequences of SEQ ID NO: 3.

22. A nucleic acid encoding the polypeptide of claim 1.

23. A nucleic acid encoding the protein of claim 11.

24. A nucleic acid encoding the protein of claim 21.

25. An AAV vector comprising a nucleic acid sequence that encodes the polypeptide of claim 1.

26. An AAV vector comprising a nucleic acid sequence that encodes the protein of claim 11.

27. An AAV vector comprising a nucleic acid sequence that encodes the protein of claim 21.

28. A method of treating an autoimmune disease in a subject comprising administering to the subject the polypeptide of claim 1.

29. A method of treating an autoimmune disease in a subject comprising administering to the subject the protein of claim 11.

30. A method of treating an autoimmune disease in a subject comprising administering to the subject the protein of claim 21.


SEQUENCE LISTING (55 sequences)

SEQ ID NO: 1 - protein, 353 aa, synthetic construct
EVQLLESGGGLVQPGGSLRLSCAASGRTFSNYHMGWFRQAPGQGREFVATIIRTGETIYYADSVKGRFTISRDNSKNTLYLQMNSLRAEDTAVYYCAAATSGWNIKTVTPYEYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 353 aa, synthetic construct
EVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSESKYGPPCPPCPAPEVEGGPSVFLFPPKPKDTLMISRTPEVTCVVVGVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLGK

SEQ ID NO: 3 - protein, 564 aa, synthetic construct
ESKYGPPCPPCPAPEVEGGPSVFLFPPKPKDTLMISRTPEVTCVVVGVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLGGGGGGSEVQLLESGGGLVQPGGSLRLSCAASGRTFSNYHMGWFRQAPGQGREFVATIIRTGETIYYADSVKGRFTISRDNSKNTLYLQMNSLRAEDTAVYYCAAATSGWNIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 4 - protein, 564 aa, synthetic construct
ESKYGPPCPPCPAPEVEGGPSVFLFPPKPKDTLMISRTPEVTCVVVGVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGSEVQLLESGGGLVQPGGSLRLSCAASGRTFSNYHMGWFRQAPGQGREFVATIIRTGETIYYADSVKGRFTISRDNSKNTLYLQMNSLRAEDTAVYYCAAATSGWNIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 5 - protein, 330 aa, synthetic construct
EVQLLESGGGLVQPGGSLRLSCAASGRTFSNYHMGWFRQAPGQGREFVATIIRTGETIYYADSVKGRFTISRDNSKNTLYLQMNSLRAEDTAVYYCAAATSGWNIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 6 - protein, 562 aa, synthetic construct
DKTHTCPPCPAPEVEGGPSVFLFPPKPKDTLMISRTPEVTCVVVGVSHEDPEVKFNWYVDGVEVHNAKTKPREEQYNSTYRVVSVLQVLHVDWLNGKEYKCKVSNKALPAPIEKTISKAKGQPREPQVYTLPPSREEMTKNQVSLTCLVKGFYPSDIVVEWESNGQPENNYKTTPPVLDSDGSFFLYSKLTVDKSRWQQGNVFSCSVMHEALHNHYTQKSLSLSPGGGGGGSEVQLLESGGGLVQPGGSLRLSCAASGRTFSNYHMGWFRQAPGQGREFVATIIRTGETIYYADSVKGRFTISRDNSKNTLYLQMNSLRAEDTAVYYCAAATSGWNIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 7 - protein, 564 aa, synthetic construct
ESKYGPPCPPCPAPEVEGGPSVFLFPPKPKDTLMISRTPEVTCVVVGVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLGGGGGGSEVQLLESGGGLVQPGGSLRLSCAASGRTFSNYHMGWFRQAPGQGREFVATIIRTGETIYYADSVKGRFTISRDNSKNTLYLQMNSLRAEDTAVYYCAAATSGWNIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDRNSTWDPPVPKCLKV

SEQ ID NO: 8 - protein, 330 aa, synthetic construct
EVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 9 - protein, 191 aa, synthetic construct
RETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 10 - protein, 124 aa, synthetic construct
EVQLLESGGGLVQPGGSLRLSCAASGRTFSNYHMGWFRQAPGQGREFVATIIRTGETIYYADSVKGRFTISRDNSKNTLYLQMNSLRAEDTAVYYCAAATSGWNIKTVTPYEYWGQGTLVTVSS

SEQ ID NO: 11 - protein, 124 aa, synthetic construct
EVQLLESGGGLVQPGGSLRLSCAASGRTFSNYHMGWFRQAPGQGREFVATIIRTGKTIYYADSVKGRFTISRDNSKNTLYLQMNSLRAEDTAVYYCAAATSGWHIKTVTPYEYWGQGTLVTVSS

SEQ ID NO: 12 - protein, 124 aa, synthetic construct
EVQLLESGGGLVQPGGSLRLSCAASGRTFSNYHMGWFRQAPGQGREFVATIIRTGTTIYYADSVKGRFTISRDNSKNTLYLQMNSLRAEDTAVYYCAAATSGWHIKTVTPYEYWGQGTLVTVSS

SEQ ID NO: 13 - protein, 124 aa, synthetic construct
EVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSS

SEQ ID NO: 14 - protein, 358 aa, synthetic construct
CEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 15 - protein, 191 aa, synthetic construct
RETCPYIRDPLNGQAVPANGTYEFGYQMHFICNEGYYLIGEEILYCELKGSVAIWRGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 16 - protein, 5 aa, synthetic construct
NYHMG

SEQ ID NO: 17 - protein, 17 aa, synthetic construct
TIIRTGETIYYADSVKG

SEQ ID NO: 18 - protein, 15 aa, synthetic construct
ATSGWNIKTVTPYEY

SEQ ID NO: 19 - protein, 7 aa, synthetic construct
GRTFSNY

SEQ ID NO: 20 - protein, 6 aa, synthetic construct
IRTGET

SEQ ID NO: 21 - protein, 17 aa, synthetic construct
TIIRTGKTIYYADSVKG

SEQ ID NO: 22 - protein, 15 aa, synthetic construct
ATSGWHIKTVTPYEY

SEQ ID NO: 23 - protein, 6 aa, synthetic construct
IRTGKT

SEQ ID NO: 24 - protein, 17 aa, synthetic construct
TIIRTGTTIYYADSVKG

SEQ ID NO: 25 - protein, 6 aa, synthetic construct
IRTGTT

SEQ ID NO: 26 - protein, 17 aa, synthetic construct
TIIRHGTTIYYADSVKG

SEQ ID NO: 27 - protein, 6 aa, synthetic construct
IRHGTT

SEQ ID NO: 28 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 231 aa, synthetic construct
DKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 30 - protein, 228 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 31 - protein, 228 aa, synthetic construct
ESKYGPPCPPCPAPEVEGGPSVFLFPPKPKDTLMISRTPEVTCVVVGVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 32 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLG

SEQ ID NO: 33 - protein, 228 aa, synthetic construct
ESKYGPPCPPCPAPEVEGGPSVFLFPPKPKDTLMISRTPEVTCVVVGVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLG

SEQ ID NO: 34 - protein, 226 aa, synthetic construct
DKTHTCPPCPAPEVEGGPSVFLFPPKPKDTLMISRTPEVTCVVVGVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 35 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSREEMTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 36 - protein, 226 aa, synthetic construct
DKTHTCPPCPAPEVEGGPSVFLFPPKPKDTLMISRTPEVTCVVVGVSHEDPEVKFNWYVDGVEVHNAKTKPREEQYNSTYRVVSVLQVLHVDWLNGKEYKCKVSNKALPAPIEKTISKAKGQPREPQVYTLPPSREEMTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 37 - protein, 5 aa, synthetic construct
GGGGS

SEQ ID NO: 38 - protein, 10 aa, synthetic construct
GGGGSGGGGS

SEQ ID NO: 39 - protein, 15 aa, synthetic construct
GGGGSGGGGSGGGGS

SEQ ID NO: 40 - protein, 20 aa, synthetic construct
GGGGSGGGGSGGGGSGGGGS

SEQ ID NO: 41 - protein, 6 aa, synthetic construct
GGGGGS

SEQ ID NO: 42 - protein, 12 aa, synthetic construct
GGGGGSGGGGGS

SEQ ID NO: 43 - protein, 559 aa, synthetic construct
EVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVESKYGPPCPPCPAPEVEGGPSVFLFPPKPKDTLMISRTPEVTCVVVGVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLGK

SEQ ID NO: 44 - protein, 559 aa, synthetic construct
EVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVESKYGPPCPPCPAPEFVGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 553 aa, synthetic construct
EVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVESKYGPPCPPCPAPEVEGGPSVFLFPPKPKDTLMISRTPEVTCVVVVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLGK

SEQ ID NO: 46 - protein, 548 aa, synthetic construct
EVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVESKYGPPCPPCPAPEVEGGPSVFLFPPKPKDTLMISRTPEVTCVVVVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLGK

SEQ ID NO: 47 - protein, 564 aa, synthetic construct
ESKYGPPCPPCPAPEVEGGPSVFLFPPKPKDTLMISRTPEVTCVVVGVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGSEVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 48 - protein, 564 aa, synthetic construct
ESKYGPPCPPCPAPEVEGGPSVFLFPPKPKDTLMISRTPEVTCVVVGVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLGGGGGGSEVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 49 - protein, 564 aa, synthetic construct
ESKYGPPCPPCPAPEFVGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGSEVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 50 - protein, 563 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGGGGGSEVQLLESGGGLVQPGGSLRLSCAASGRTFSNYHMGWFRQAPGQGREFVATIIRTGETIYYADSVKGRFTISRDNSKNTLYLQMNSLRAEDTAVYYCAAATSGWNIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 51 - protein, 558 aa, synthetic construct
EVQLLESGGGLVQPGGSLRLSCAASGRTFSNYHMGWFRQAPGQGREFVATIIRTGETIYYADSVKGRFTISRDNSKNTLYLQMNSLRAEDTAVYYCAAATSGWNIKTVTPYEYWGQGTLVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVESKYGPPCPPCPAPEVEGGPSVFLFPPKPKDTLMISRTPEVTCVVVVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLGK

SEQ ID NO: 52 - protein, 554 aa, synthetic construct
EVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSESKYGPPCPPCPAPEVEGGPSVFLFPPKPKDTLMISRTPEVTCVVVGVSQEDPEVQFNWYVDGVEVHNAKTKPREEQFNSTYRVVSVLQVLHVDWLNGKEYKCKVSNKGLPSSIEKTISKAKGQPREPQVYTLPPSQEEMTKNQVSLTCLVKGFYPSDIVVEWESNGQPENNYKTTPPVLDSDGSFFLYSRLTVDKSRWQEGNVFSCSVMHEALHNHYTQKSLSLSLGKGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 53 - protein, 564 aa, synthetic construct
ESKYGPPCPPCPAPEVEGGPSVFLFPPKPKDTLMISRTPEVTCVVVGVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGSEVQLQESGGGLVQPGGSLRLSCAASGRTFSNYHMGWFRQAPGKEREFVGTIIRTGETIYYADSVKGRFTISRDNDKNTVYLQMNSLKPGDTAVYYCAAATSGWNIKTVTPYEYWGQGTQVTVSSGGGGSGGGGSGGGGSRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 54 - protein, 565 aa, synthetic construct
ESKYGPPCPPCPAPEVEGGPSVFLFPPKPKDTLMISRTPEVTCVVVGVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGSEVQLLESGGGLVQPGGSLRLSCAASGRTFSNYHMGWFRQAPGQGREFVATIIRHGTTIYYADSVKGRFTISRDNSKNTLYLQMNSLRAEDTAVYYCAAATSGWHIKTVTPYEYWGQGTLVTVSSGGGGSGGGGSGGGGSRRKACSNLPDPLNGQVSYPNGDMLFGSKAQFTCNTGFYIIGAETVYCQVSGNVMAWSEPSPLCEKILCKPPGEIPNGKYTNSHKDVFEYNEVVTYSCLSSTGPDEFSLVGESSLFCIGKDEWSSDPPECKVVKCPYPVVPNGEIVSGFGSKFYYKAEVVFKCNAGFTLHGRDTIVCGANSTWEPEMPQCIKD

SEQ ID NO: 55 - protein, 124 aa, synthetic construct
EVQLQESGGGLVQPGGSLRLSCAASGRTFSNYHMGWFRQAPGKEREFVGTIIRTGETIYYADSVKGRFTISRDNDKNTVYLQMNSLKPGDTAVYYCAAATSGWNIKTVTPYEYWGQGTQVTVSS